(19) United States
(12) Patent Application Publication
(10) Pub. No.: US 2025/0308666 A1
(43) Pub. Date: Oct. 2, 2025

(54) SYSTEMS AND METHODS FOR MEDICATION DOSING AND TITRATION

(71) Applicant: Abbott Diabetes Care Inc., Alameda, CA (US)

(72) Inventors: Aparajita BHATTACHARYA, Dublin, CA (US); Kenneth J. DONIGER, Menlo Park, CA (US); Erwin Satrya BUDIMAN, Fremont, CA (US); Gary A. HAYTER, Oakland, CA (US); Roman HOVORKA, St. Albans (GB)

(21) Appl. No.: 19/094,102

(22) Filed: Mar. 28, 2025

Related U.S. Application Data
(60) Provisional application No. 63/571,210, filed on Mar. 28, 2024.

(51) Int. Cl. G16H 20/17; A61B 5/00; A61B 5/145

(52) U.S. Cl. CPC G16H 20/17; A61B 5/14503; A61B 5/14532; A61B 5/7264; A61B 5/7275; A61B 5/7435; A61B 5/7475; A61B 2560/0462

(57) ABSTRACT

Dose guidance systems and methods for titrating medication doses are described. The dose guidance system may receive glucose data from a continuous glucose monitor and may receive medication data related to medication administered by the user. The dose guidance system may initialize dose guidance parameters, recommend medication doses, titrate medication doses, and provide alerts based on the glucose data and medication data.

This application claims the benefit of U.S. Provisional Application No. 63/571,210, filed Mar. 28, 2024, which application is incorporated herein by reference in its entirety.

### FIELD OF THE INVENTION

Embodiments described herein relate to dose guidance systems for titrating medication doses and providing medication dose recommendations.

### BACKGROUND

Patients with diabetes or other illnesses monitor analyte levels to help maintain analyte levels in a target range. For example, patients with diabetes monitor glucose levels to maintain glucose levels in a target range and to avoid hypoglycemia or hyperglycemia. Failure to maintain analyte levels within a target range can result in serious health complications. By monitoring analyte levels, the patient can determine the impact of food, exercise, and medication on the patient's analyte levels. Further, the patient can more easily determine when analyte levels are not in range so that the patient may take action, such as to administer medication, eat a meal, or adjust other self-care behaviors.

Analyte data may also be used by healthcare professionals to help guide the patient's treatment. The patient may administer insulin in one or more doses throughout the day to lower glucose levels. However, it is important to determine the proper doses to administer to avoid hypoglycemia. As a result, patients with diabetes carefully monitor glucose levels and administer medication to maintain glucose levels in the target range. This can be a time-consuming and burdensome process, and interpreting analyte data and determining the proper dose of medication to administer can be challenging for both the patient and healthcare professional. Accordingly, there is a need for improved systems and methods for determining and recommending doses of medication to administer.

### SUMMARY OF THE INVENTION

Described herein is a method for titrating a medication dose. The method includes receiving, by a processor, glucose data collected by a glucose monitoring device worn by a user over each of a plurality of time of day periods, wherein the glucose monitoring device is in communication with the processor. The method further includes determining, by the processor, a time of administration of each of a plurality of medication doses taken by the user. The method includes detecting, by the processor, a fasting period based on one or more of the glucose data and the times of administration of the plurality of medication doses. The method includes determining, by the processor, a change in a schedule of a user based on a detected change in the fasting period; adjusting, by the processor, the plurality of time of day periods based on the determined change in the schedule of the user; and titrating the medication doses for each time of day period of the adjusted plurality of time of day periods.

In any of the various embodiments described herein, the fasting period may correspond to a longest interval between consecutive medication doses of the plurality of medication doses.

In any of the various embodiments described herein, detecting the fasting period may include determining a variability in the glucose data for a plurality of moving windows of time, and the fasting period may be a moving window of time of the plurality of moving windows of time having the lowest glucose variability.

In any of the various embodiments described herein, detecting the change in the fasting period may include detecting the start time of the fasting period has changed from a first time to a second time, and the start time of the fasting period is the second time for a predetermined minimum period of time.

In any of the various embodiments described herein, detecting the fasting period may include autocorrelation or cross-correlation of the glucose data.

In any of the various embodiments described herein, the glucose monitoring device may include an in vivo glucose sensor comprising a first portion positioned below the skin and in contact with a bodily fluid and a second portion positioned above the skin. In any of the various embodiments described herein, the medication doses may include insulin doses, wherein the insulin doses comprise fast-acting insulin, slow-acting insulin, or pre-mixed insulin.

In any of the various embodiments described herein, the plurality of time of day periods may include a plurality of mealtime periods.

In any of the various embodiments described herein, the plurality of time of day periods may include a breakfast period, a lunch period, a dinner period, and an overnight period.

Some embodiments described herein relate to a method for titrating a medication dose that includes receiving, by one or more processors, glucose data from a continuous glucose monitoring device over a predetermined period of time, and receiving, by the one or more processors, medication data including a time and an amount of one or more medication doses. The method includes segmenting, by the one or more processors, the glucose data into a plurality of glucose data segments, wherein the glucose data segments include glucose data segments associated with medication doses; performing, by the one or more processors, a glucose pattern analysis for one or more glucose data segments of the plurality of glucose data segments; and performing, by the one or more processors, an event counting analysis on one or more of the plurality of glucose data segments. The method further includes determining, by the one or more processors, to increase, decrease or maintain the medication dose based on the glucose pattern analysis and the event counting analysis for the associated glucose data segment; and outputting, on a display device in communication with the one or more processors, a recommendation to increase, decrease or maintain the medication dose.

In any of the various embodiments described herein, the glucose data segments may include a meal segment, wherein the meal segment may include a start time corresponding to a time of administration of a medication dose, and an end time that is one of a start time of a subsequent medication dose or a fixed amount of time following the start time. The glucose data segments may include an idle segment, wherein the idle segment may have a start time at an end of the fixed amount of time following the start time of the meal segment and that has an end time at a start time of a subsequent medication dose. The event counting analysis may include counting a number of low glucose events or low glucose alarms in each of the meal segments and the idle segments. Titrating the medication dose may include decreasing a basal dose if the number of low glucose events or low glucose alarms in one or more of the idle segments exceeds a threshold number of low glucose events. Titrating the medication dose may include decreasing a medication dose associated with a meal if the number of low glucose events or low glucose alarms in the associated glucose data segment exceeds a threshold number of low glucose events or low glucose alarms.

In any of the various embodiments described herein, the method may further include determining an overnight period from the idle segment when a pattern of glucose data segments includes an idle segment followed by a breakfast segment, and wherein the overnight period has an end time at the end of the idle segment and a start time that is a fixed number of hours before the end time of the overnight period. The glucose pattern analysis may include determining a glucose pattern for each of the meal segments and the overnight glucose data segments.

In any of the various embodiments described herein, the method may further include determining validity of the glucose data segments prior to the event counting analysis or the pattern analysis. Determining the validity of the glucose data segments may include deeming glucose data segments invalid that have a start time that is more than a predetermined amount of time before a most recent glucose data segment.

In any of the various embodiments described herein, the event counting analysis may include determining for each meal segment a number of correction doses following the meal segment. The method may further include adjusting a glucose pattern for the meal segment if the number of correction doses following the meal segment exceeds a threshold number of correction doses.

In any of the various embodiments described herein, the method may further include determining a medication dose of the one or more medication doses is a meal dose when the dose amount corresponds to a recommended dose amount.

In any of the various embodiments described herein, the method may further include determining a medication dose of the one or more medication doses is a meal dose when a time of administration of the medication dose is within a predetermined period of time from a recommendation for the meal dose.

In any of the various embodiments described herein, the method may further include classifying a medication dose as a breakfast, lunch, or dinner dose, respectively, based on the time of administration of the dose being within a predetermined time range for one of the breakfast, lunch, or dinner doses.

Some embodiments described herein relate to a method of titrating a medication dose that includes receiving, by a processor, glucose data collected by a glucose monitoring device comprising a first portion positioned under a skin surface of a user and in contact with a bodily fluid; and determining, by the processor, a glucose median for a time of day period based on the glucose data collected by the glucose monitoring device. The method further includes determining, by the processor, a hypoglycemic risk based on comparison of a glucose median of a user to a hypoglycemia risk threshold for the time of day period; and outputting, by the processor, a recommendation to increase the medication dose by an amount based on the hypoglycemic risk.

In any of the various embodiments described herein, the recommended increase in the medication dose may be proportional to the hypoglycemic risk.

In any of the various embodiments described herein, the method may further include categorizing the determined hypoglycemic risk into a first bin when the hypoglycemic risk is below a hypoglycemic risk threshold or a second bin when the hypoglycemic risk is above the hypoglycemic risk threshold, and the amount is a first amount when the hypoglycemic risk is categorized into the first bin, and the amount is a second amount that is greater than the first amount when the hypoglycemic risk is categorized into the second bin.

In any of the various embodiments described herein, the hypoglycemic risk may be a vertical distance of a glucose median to a hypoglycemia risk curve on a plot of glucose median over glucose variability, wherein the hypoglycemia risk curve includes a set of points having the same value for a hypoglycemia risk metric.

In any of the various embodiments described herein, the amount of increase may be a percentage of the medication dose.

In any of the various embodiments described herein, the amount of increase may be a number of units by which to increase the medication dose.

In any of the various embodiments described herein, the method may further include determining a hypoglycemic risk for a first meal period and for a second meal period following the first meal period, and recommending an increase in the medication doses for each of the first and the second meal periods when the hypoglycemic risk of the second meal period is greater than the hypoglycemic risk of the first meal period.

Some embodiments described herein relate to a method for titrating an insulin dose that includes receiving, by a processor, medication information comprising time of administration of one or more medication doses, and receiving low glucose event data from a continuous glucose monitor in communication with the processor. The method further includes associating, by the processor, a low glucose event with a meal dose if the low glucose event occurs within a first period of time following administration of the meal dose; associating a low glucose event with a basal dose when the low glucose event does not occur within the first period of time, and the low glucose event occurs within a second period of time following the administration of the basal dose; and recommending a decrease in the associated meal dose or basal dose if the number of low glucose events exceeds a threshold number of low glucose events for the meal dose or the basal dose.

In any of the various embodiments described herein, the low glucose event data may include low glucose events in which a predetermined minimum number of glucose readings are below a predetermined low glucose level in a predetermined period of time.

In any of the various embodiments described herein, the low glucose event data may include low glucose alarms output by the continuous glucose monitor.

In any of the various embodiments described herein, the second period of time may be 24 hours.

In any of the various embodiments described herein, the threshold number of low glucose events may differ for the meal dose and for the basal dose.

Some embodiments described herein relate to a method of titrating a medication dose that includes receiving, by a processor, glucose data from a glucose monitoring device worn by a user; and receiving, by the processor, medication data relating to medication doses taken by the user. The method further includes identifying, by the processor, a glucose pattern for each of a plurality of time of day periods based on the glucose data; and determining, by the processor, to decrease a medication dose for a time of day period of the plurality of time of day periods based on the identified glucose pattern, wherein the determination to decrease the medication dose is based on a first number of days of glucose data in each of the plurality of time of day periods. The method further includes determining, by the processor, to increase a medication dose for a time of day period of the plurality of time of day periods based on the identified glucose pattern, wherein the determination to increase the medication dose is based on a second number of days of glucose data in each of the plurality of time of day periods, wherein the second number of days is greater than the first number of days. The method further includes outputting, on a display of a display device in communication with the processor, a recommendation to decrease or increase the medication dose.

In any of the various embodiments described herein, the plurality of time of day periods may correspond to mealtimes.

In any of the various embodiments described herein, the medication doses may include one or more of rapid-acting insulin doses or long-acting insulin doses.

Some embodiments described herein relate to a method of titrating an insulin dose that includes receiving, by a processor, glucose data from a glucose monitoring device worn by a user; and receiving, by the processor, medication data relating to insulin doses administered by the user. The method further includes identifying, by the processor, a glucose pattern for each of a plurality of time of day periods based on the glucose data; and determining, by the processor, to increase a rapid-acting insulin dose for a first time of day period of the plurality of time of day periods based on the identified glucose pattern when a minimum number of days of glucose data is available for the first time of day period and for a consecutive time of day period. The method further includes determining, by the processor, to increase a long-acting insulin dose based on the identified glucose patterns for the plurality of time of day periods when the minimum number of days of glucose data is available for each of the plurality of time of day periods. The method further includes outputting, on a display of a display device in communication with the processor, a recommendation to increase the rapid-acting insulin dose or the long-acting insulin dose.

In any of the various embodiments described herein, the plurality of time of day periods may correspond to mealtimes.

In any of the various embodiments described herein, the minimum number of days of glucose data may be in a range of 5 to 10 days.

Some embodiments described herein relate to a method of titrating parameters of a dose guidance system, that includes receiving, at an input of a display device, a user entered value for a first parameter of the dose guidance system; and selecting, by the dose guidance system, a value for a second parameter based on the value entered for the first parameter. The method further includes titrating one or more of the first parameter and second parameter based on the glucose data of the user; and determining if a titration limit for the first parameter or the second parameter is reached. The method further includes outputting a notification when the titration limit has been reached.

In any of the various embodiments described herein, the first parameter and the second parameter may be titrated independently of one another.

In any of the various embodiments described herein, the second parameter may be in a fixed relationship to the first parameter.

In any of the various embodiments described herein, the first parameter may be a pre-meal correction factor and the second parameter may be a post-meal correction factor. In any of the various embodiments described herein, the method may further include receiving user entry of the titration limit.

In any of the various embodiments described herein, the titration limit may be a total daily dose.

In any of the various embodiments described herein, the first parameter may have a first titration limit and the second parameter may have a second titration limit. The first titration limit may be a maximum total daily dose, and the second parameter may be a minimum correction factor.

In any of the various embodiments described herein, outputting a notification when the titration limit has been reached may include outputting an alert on a display device of the user.

In any of the various embodiments described herein, outputting a notification when the titration limit has been reached may include providing a notification in a dose guidance status section of a user interface.

Some embodiments described herein relate to a method of notifying a user of a late medication dose that includes receiving, by a processor, glucose data from a glucose monitoring device in wireless communication with the processor; and detecting, by the processor, a meal based on the glucose data received from the glucose monitoring device. The method further includes outputting, by a display device in communication with the processor, a notification when the meal is detected during a predetermined late dose period, wherein the notification alerts the user of the late medication dose; and recommending an insulin dose during the predetermined late dose period.

In any of the various embodiments described herein, the recommended insulin dose may be based on the glucose level at a start time of the detected meal.

In any of the various embodiments described herein, the method may further include stopping output of the notification when the predetermined late dose period has elapsed.

In any of the various embodiments described herein, the method may further include outputting a second notification at the end of the predetermined late dose period, wherein the second notification indicates that the insulin dose is no longer recommended.

In any of the various embodiments described herein, the method may further include stopping output of the notification when an insulin dose is administered.

In any of the various embodiments described herein, the method may further include receiving user input declining the recommended medication dose, and stopping outputting the notification when the user input declining the recommended medication dose is received.

In any of the various embodiments described herein, the predetermined late dose period may be in a range of 1 to 3 hours.

Some embodiments described herein relate to a method of setting an initial value for rapid-acting insulin doses in a dose guidance system that includes receiving, by a processor, glucose data collected by a glucose monitoring device during a learning period; and receiving, by the processor, medication data collected during the learning period, wherein the medication data comprises an amount and a time of administration for each of a plurality of rapid-acting doses. The method further includes classifying, by the processor, the rapid-acting doses as one of a plurality of mealtime doses; and extracting, by the processor, one or more glucose features from post-prandial glucose data following each of the one or more mealtime doses. The method further includes adjusting, by the processor, the mealtime doses to optimize the one or more glucose features; and initializing, by the processor, each of the mealtime doses in the dose guidance system based on the adjusted mealtime doses.

In any of the various embodiments described herein, the plurality of mealtime doses may include a breakfast dose, a lunch dose, and a dinner dose.

In any of the various embodiments described herein, classifying the rapid-acting doses into the plurality of mealtime doses may include a cluster analysis based on the time of administration of each of the rapid-acting doses during the learning period. A median time of administration of a cluster of rapid-acting doses may be determined as the mealtime. The post-prandial glucose data may include a predetermined period following the mealtime.

In any of the various embodiments described herein, the one or more glucose features may include a time in range.

In any of the various embodiments described herein, the one or more glucose features may include a hypoglycemia metric.

In any of the various embodiments described herein, the post-prandial glucose data may include glucose data in a predetermined period of time following the mealtime, wherein the predetermined period of time is 2 hours.

Described herein is a method of setting an initial value for a basal insulin dose in a dose guidance system that includes receiving, by a processor, glucose data collected by a glucose monitoring device during a learning period; and receiving, by the processor, medication data collected during the learning period, wherein the medication data includes a plurality of basal insulin doses. The method further includes extracting, by the processor, one or more glucose features from a period of time following administration of each basal insulin dose; adjusting, by the processor, a basal insulin dose amount to optimize the one or more glucose features; and initializing, by the processor, the basal insulin dose in the dose guidance system based on the adjusted basal insulin dose amount. Some embodiments described herein relate to a method of titrating a medication dose that includes receiving glucose data from a glucose monitoring device worn on a body of a user, wherein the glucose data is received following administration of a medication dose to the user. The method further includes determining to increase, decrease, or maintain an amount of the medication dose based on the glucose data. When determining to decrease the amount of the medication dose, determining an amount of a next medication dose includes: setting the medication dose that resulted in a recommendation to decrease the medication dose as an upper limit, setting a largest medication dose that resulted in a recommendation to increase the medication dose as a lower limit, and recommending a next medication dose that is in a range of the upper limit and the lower limit.

In any of the various embodiments described herein, the recommended dose of the next medication dose may be an average of the upper limit and the lower limit. In any of the various embodiments described herein, the method may further include determining one or more glucose metrics and one or more medication metrics, and determining the medication dose is fully titrated when a glucose metric of the one or more glucose metrics or a medication metric of the one or more medication metrics meets or exceeds a threshold level. The one or more glucose metrics may include a time in range, and the medication dose is determined to be fully titrated when the time in range is within a predetermined percentage of a maximum time in range. The maximum time in range may be determined based on a relationship of time in range and a standard deviation of the glucose levels of the user over a period of time. The one or more glucose metrics may include a glucose median, and wherein the medication dose is determined to be fully titrated when the glucose median is within a predetermined percentage of a target glucose median. The one or more medication metrics may include a ratio of dose per body weight, and the medication dose is determined to be fully titrated when the ratio of dose per body weight meets or exceeds a threshold ratio of dose to body weight. The one or more glucose metrics may include a difference between a glucose level at midnight and a lowest overnight glucose level, and the medication dose is determined to be fully titrated when the difference is greater than a difference threshold.

Described herein is a method of titrating an insulin dose that includes receiving, by a processor, glucose data from a continuous glucose monitoring device worn on a body of a user collected over a predetermined period of time, and receiving, by the processor, medication data relating to insulin doses administered by the user over the predetermined period of time. The method further includes determining an optimal dose based on the glucose data, and determining an amount of a dose change to a current medication dose based on a difference between the current dose and the optimal dose.

In any of the various embodiments described herein, the optimal dose may be based on a difference between a current glucose median and a goal median. The goal median may be a difference between the current glucose median and a margin, wherein the margin may be a difference between a low percentile glucose level and a hypoglycemia threshold. The low percentile glucose level may be a 4^{th} percentile glucose value.

In any of the various embodiments described herein, the optimal dose may be further based on a median sensitivity. The median sensitivity may be based on population data. The median sensitivity may be determined based a linear fit of data points indicative of the glucose median of the user following administration of a basal insulin dose.

In any of the various embodiments described herein, the method may further include adjusting the amount of the dose change based on a safety factor.

Described herein is a method of titrating a medication dose that includes titrating, by a processor, a medication dose based on glucose data from a first preceding period of time, wherein the glucose data is received from a glucose monitoring device in communication with the processor. The method further includes determining, by the processor, that the medication dose is fully titrated based on one or more full titration criteria; and titrating, by the one or more processors, the medication dose based on glucose data collected over a second preceding period of time when the medication dose is fully titrated as determined based on the one or more full titration criteria, wherein the second preceding period of time is longer than the first preceding period of time.

In any of the various embodiments described herein, the method may further include outputting, by a display device in communication with the processor, a notification when a full titration criterion of the one or more full titration criteria are satisfied.

In any of the various embodiments described herein, the method may further include stopping titration of the medication dose when a risk of hypoglycemia is detected. The risk of hypoglycemia may be based on a time below a predetermined low glucose value exceeding a threshold amount of time.

In any of the various embodiments described herein, the method may further include determining that a new therapy is initiated, and stopping titration of the medication dose when the new therapy is initiated. Determining that the new therapy is initiated may include receiving user input indicating initiation of a second medication.

In any of the various embodiments described herein, the method may further include determining the medication dose is fully titrated when the medication dose has not changed over a predetermined period of time.

In any of the various embodiments described herein, determining the medication dose is fully titrated may include determining a glucose pattern for each of a plurality of time of day periods, and determining the medication dose is fully titrated when the determined glucose patterns for the time of day periods have not changed over a predetermined period of time.

In any of the various embodiments described herein, determining the medication dose is fully titrated may include determining that a change in time in range from a first medication dose to a second medication dose resulted in a change in time in range of less than a predetermined change in time in range threshold.

In any of the various embodiments described herein, determining the medication dose is fully titrated may include determining that a minimum 4^{th} percentile glucose value is less than a predetermined threshold.

### BRIEF DESCRIPTION OF THE FIGURES/DRAWINGS

The accompanying drawings, which are incorporated herein and form a part of the specification, illustrate the present disclosure and, together with the description, further serve to explain the principles thereof and to enable a person skilled in the pertinent art to make and use the same.

FIGS. 1A and 1B are block diagrams of exemplary dose guidance systems.

FIG. 2A shows a schematic diagram depicting a sensor control device according to an embodiment.

FIG. 2B shows a block diagram depicting an example embodiment of a sensor control device.

FIG. 3A shows a schematic diagram depicting an example embodiment of a display device.

FIG. 3B shows a block diagram depicting an example embodiment of a display device.

FIG. 4 shows an example embodiment of a graph depicting information for determining a hypoglycemia risk and other metrics for a glucose pattern analysis.

FIG. 5 shows a method for determining time of day periods for use in a dose guidance algorithm according to an embodiment.

FIG. 6 shows a method for determining time of day periods for use in a dose guidance algorithm according to an embodiment.

FIG. 7A shows an example of time-series of glucose data over a time of day.

FIG. 7B shows an autocorrelation of the glucose data of FIG. 7A.

FIG. 7C shows a median glucose level over time of day based on the time-series of glucose data of FIG. 7A.

FIG. 8 shows a schematic diagram of a dose guidance algorithm including glucose data segmentation according to an embodiment.

FIG. 9 shows an example of glucose data segmentation according to the dose guidance algorithm of FIG. 8.

FIGS. 10A and 10B show examples of glucose data segments assessed for validity and subject to event counting and pattern analyses according to the dose guidance algorithm of FIG. 8.

FIG. 11 shows a plot of glucose median over variability illustrating a Grisk value according to an embodiment.

FIG. 12 shows a flow chart illustrating steps of titrating a medication dose based on low glucose events according to an embodiment.

FIG. 13 shows a flow chart illustrating steps of a rapid titration algorithm according to an embodiment.

FIG. 14 shows a flow chart illustrating steps of a rapid titration algorithm according an embodiment.

FIGS. 15A and 15B show exemplary user interfaces for a dose guidance application for a display device of a user.

FIG. 16 shows an exemplary user interface for configuring a dose guidance system.

FIG. 17 shows an exemplary method of configuring a dose guidance system

FIG. 18A shows a user interface for HCPs that provides dose guidance statuses for patients according to an embodiment.

FIG. 18B shows the user interface of FIG. 16 including a notification to adjust a dose setting.

FIG. 19 shows an exemplary method of providing a late medication dose notification according to an embodiment.

FIG. 20 shows an exemplary method of providing a late medication dose notification according to an embodiment.

FIG. 21 shows an exemplary method of initializing dose amounts for a dose guidance system based on data collected during a learning period.

FIG. 22 shows an exemplary method of setting an initial value for a basal dose in a dose guidance system based on data collected during a learning period.

FIG. 23 shows a graphical representation of the margin and goal median on a plot glucose data over a 24-hour period based on glucose data collected over a plurality of days.

FIG. 24 shows a graph of different percentage time below range values plotted on a graph of glucose median over standard deviation.

FIG. 25 shows an exemplary plot of overnight glucose median over the basal dose amount.

FIG. 26 shows an exemplary method of titrating a medication dose according to an embodiment.

FIG. 27 shows an exemplary method for titrating a medication dose according to an embodiment.

FIG. 28 shows a plot of time in range vs. standard deviation based on simulated data.

FIG. 29 shows an exemplary method of determining full titration of a medication dose according to an embodiment.

FIG. 30 shows an exemplary method of titrating a medication dose according to an embodiment.

FIG. 31 shows a dose monitoring device configured as a smart pen cap according to an embodiment.

FIG. 32 shows a dose monitoring device configured as a smart pen cap according to an embodiment.

FIGS. 33A-C show an exemplary user interface of a glucose monitoring application for providing dose guidance according to an embodiment.

FIG. 34 shows a diagram of a plurality of user states corresponding to time of day periods, and a probability of the user transitioning between the time of day periods.

FIG. 35 shows plots of a probability of a user state over time for each of a plurality of time of day periods.

FIG. 36 shows an exemplary method of titrating a medication dose using glucose data segmentation.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein comport with standards used by those experienced or skilled in the art to most effectively convey the substance of their work to others skilled in the art. In some instances, well-known methods, procedures, and components have not been described in detail to avoid unnecessarily obscuring aspects of the disclosure.

References in the specification to “some embodiments” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to apply such feature, structure, or characteristic in connection with other embodiments whether or not explicitly described.

The following examples are illustrative, but not limiting, of the present disclosure. Other suitable modifications and adaptations of the variety of conditions and parameters normally encountered in the field, and which would be apparent to those skilled in the art, are within the spirit and scope of the disclosure.

Patients with diabetes may take one or more medications to help manage diabetes and control glucose levels. Determining the proper number, timing, and amount of medication doses can be difficult. The patient's health care professional (HCP) may determine the patient's medication regimen based in part on the user's glucose data, and the user's past medication data. However, HCPs may lack the time and specialized training needed to interpret voluminous continuous glucose monitoring (CGM) data and to use the data to determine whether to increase, decrease or maintain the patient's current medication doses and/or whether to escalate therapy and introduce or change medication. Interpreting CGM data can be difficult as numerous factors may impact the patient's glucose levels, such as food, meals, alcohol, activity (e.g., exercise), sleep, illness, and medications. Further, each patient's response to a medication may be different and may vary based on patient-specific factors, such as the patient's age, weight, co-morbidities, and other medications, among other factors. Patients may also not comply with the recommended medication doses, whether intentionally or unintentionally, and may occasionally miss a dose, take the wrong dose, or incorrectly record the doses administered. This can further complicate the HCP's determination of how and whether to adjust medication doses.

Further, the HCP may have limited time to review the CGM and medication data and determine the appropriate response. The HCP and patient may have an appointment on an average of once every several months. As a result, if the patient's medication therapy is not adequate, the patient may experience poor glucose control for an extended period of time, and improvements may occur very gradually. Accordingly, there is a need for improved methods and systems for providing medication dose guidance and for titrating medication doses to aid the HCP and patient in determining the proper medication therapy for the patient.

As used herein, a medication may include insulin, such as basal or long-acting insulin. Basal insulin is typically taken once per day, though in some cases may be split into two or more doses. Medication may include rapid-acting or “meal-time” insulin. Rapid-acting insulin may be taken with one or more of the main meals in the day, such as breakfast, lunch, or dinner. Rapid-acting insulin may be taken in addition to basal insulin. Medication may include pre-mixed insulin, which may include a combination of rapid-acting and long-acting insulins. Additional non-insulin medications may be taken to help to manage diabetes, including but not limited to exenatide, metformin, SGLT inhibitors, DPP-4 inhibitors, and glucagon-like peptide-1 receptor agonists (GLP-1 RA). While the disclosure herein may refer primarily to a particular type of medication, such as insulin, it is understood that the systems and methods disclosed herein may be used for other medications, such as those listed above, except where specifically indicated.

Some embodiments described herein relate to dose guidance systems or dose guidance algorithms for recommending a dose of medication for the user to administer and/or for titrating a dose of medication. The dose guidance system may include algorithms or software for providing dose recommendations and for titrating medication doses. The algorithms may include instructions stored in memory and executed by one or more processors coupled to or in communication with the memory. The algorithms may be executed on one or more processors of a display device of a user, such as a mobile device, e.g., a smartphone, a dedicated handheld display device associated with an analyte sensor, among other portable electronic devices, and other computing devices. The dose guidance algorithms may be included in a mobile application with a graphical user interface on a display of the display device. The dose guidance algorithms may be executed at a remote computer or server, and may communicate with the display device to provide medication information, such as dose recommendations. The dose guidance algorithms may be executed on a medication delivery device, such as a pump or injection pen or associated controller or display, or dose monitoring device, such as a smart pen cap, among others. The dose guidance algorithms may be executed by a combination of a display device, medication delivery device, and remote computer or server.

The dose guidance system may include one or more dose guidance algorithms as described herein. For example, a dose guidance algorithm may be based on American Diabetes Association (ADA) guidelines (e.g., may implement the ADA standards or guidelines). Another dose guidance algorithm may be personalized to the user, and may include user-defined values or values that are dynamically determined over time based on the user's glucose and insulin data. Another dose guidance algorithm may be based on machine learning or artificial intelligence. The user, such as a patient or HCP, may select a dose guidance algorithm for use by the patient. The selection may be based on the patient's goals, such as for conservative or aggressive treatment. Dose guidance system may begin with a first algorithm and transition to a second algorithm over time.

Some embodiments described herein relate to dose guidance functionality provided by dose guidance system 100. The dose guidance functionality may be implemented as a set of software instructions stored and/or executed on one or more electronic devices. This dose guidance functionality may be referred to as a dose guidance algorithm or dose guidance application. The dose guidance application may be stored, executed, and presented to the user on the same single electronic device. In other embodiments, the dose guidance application can be stored and executed on one device, and presented to the user on a different electronic device. For example, the dose guidance application can be stored and executed on trusted computer system and presented to the user by way of a webpage displayed through an internet browser executed on display device 120.

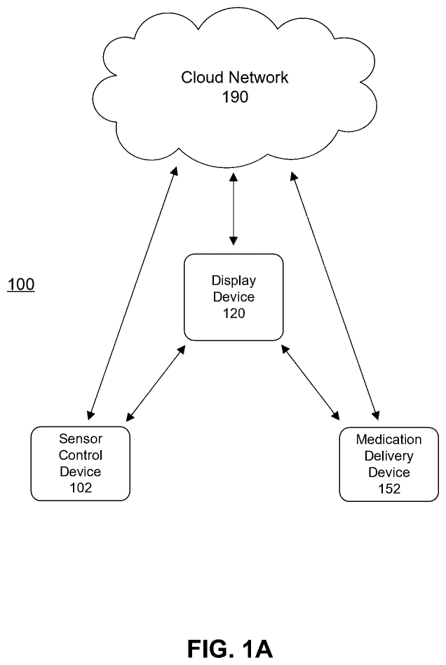

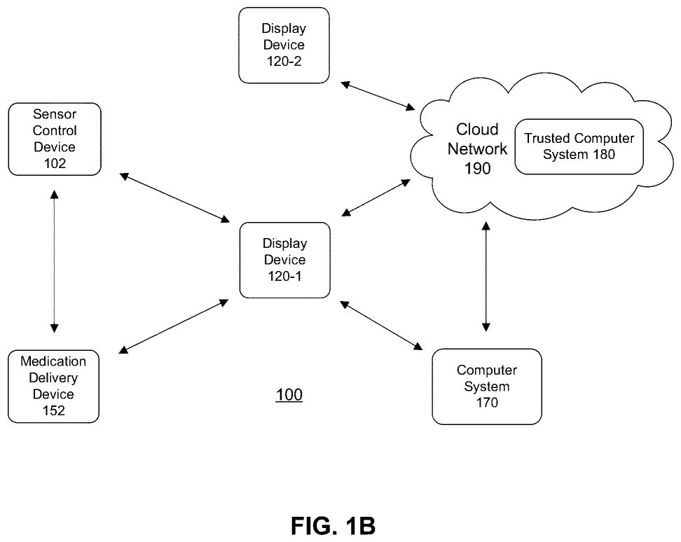

FIG. 1A is a block diagram depicting an example embodiment of dose guidance system 100. In this embodiment, dose guidance system 100 is capable of providing dose guidance, monitoring one or more analytes, and delivering one or more medications. This multifunctional example is used to illustrate the high degree of interconnectivity and performance obtainable by system 100. However, in the embodiments described herein, the analyte monitoring components, the medication delivery components, or both can be omitted if desired.

System 100 may include one or more of a sensor control device 102 configured to collect analyte level information from a user, a medication delivery device 152 configured to deliver medication to the user, and a display device 120 configured to present information to the user and receive input or information from the user. The structure and function of each device will be described in further detail herein.

System 100 is configured for highly interconnected and highly flexible communication between devices. Each of the three devices 102, 120, and 152, can communicate directly with each other (without passing through an intermediate electronic device) or indirectly with each other (such as through cloud network 190, or through another device and then through network 190). Bidirectional communication capability between devices, as well as between devices and network 190, is shown in FIG. 1A with a double-sided arrow. However, those of skill in the art will appreciate that any of the one or more devices can be capable of unidirectional communication such as, for example, broadcasting, multicasting, or advertising communications. In each instance, whether bidirectional or unidirectional, the communication can be wired or wireless. The protocols that govern communication over each path can be the same or different, and can be either proprietary or standardized. For example, wireless communication between devices 102, 120, and 152 can be performed according to a Bluetooth (including Bluetooth Low Energy) standard, a Near Field Communication (NFC) standard, a Wi-Fi (802.11x) standard, a mobile telephony standard, or others. All communications over the various paths can be encrypted, and each device of FIG. 1A can be configured to encrypt and decrypt those communications sent and received. In each instance the communication pathways of FIG. 1A can be direct (e.g., Bluetooth or NFC) or indirect (e.g., Wi-Fi, mobile telephony, or other internet protocol). Embodiments of system 100 do not need to have the capability to communicate across all of the pathways indicated in FIG. 1A.

In addition, although FIG. 1A depicts a single display device 120, a single sensor control device 102, and a single medication delivery device 152, those of skill in the art will appreciate that system 100 can comprise a plurality of any of the aforementioned devices. By way of example only, system 100 can comprise a single sensor control device 102 in communication with multiple (e.g., two, three, four, etc.) display devices 120 and/or multiple medication delivery devices 152. Alternatively, system 100 can comprise a plurality of sensor control devices 102 in communication with a single display device 120 and/or a single medication delivery device 152. Furthermore, each of the plurality of devices can be of the same or different device types. For example, system 100 can comprise multiple display devices 120, including a smart phone, a handheld receiver, and/or a smart watch, each of which can be in communication with sensor control device 102 and/or medication delivery device 152, as well in communication with each other (e.g., via Bluetooth or other wireless communication methods). Any of sensor control device 102, display device 120, medication delivery device 152 or cloud network 190, or combinations thereof, may store and/or execute dose guidance algorithms as discussed herein.

Analyte data and other data can be transferred between each device within system 100 in an autonomous fashion (e.g., transmitting automatically according to a schedule), or in response to a request for analyte data (e.g., sending a request from a first device to a second device for analyte data, followed by transmission of the analyte data from the second device to the first device). Other techniques for communicating data can also be employed to accommodate more complex systems like cloud network 190.

FIG. 1B is a block diagram depicting another example embodiment of dose guidance system 100. Here, system 100 includes sensor control device 102, medication delivery device 152, a first display device 120-1, a second display device 120-2, local computer system 170, and trusted computer system 180 that is accessible by cloud network 190. Dose guidance algorithms as disclosed herein may be stored and/or executed by any of sensor control device 102, medication delivery device 152, first display device 120-1, second display device 120-2, local computer system 170, trusted computer system 180, or cloud network 190, or combinations thereof. Sensor control device 102 and medication delivery device 152 are capable of communication with each other and with display device 120-1, which can act as a communication hub for aggregating information from sensor control device 102 and medication delivery device 152, processing and displaying that information where desired, and transferring some or all of the information to cloud network 190 and/or computer system 170. Conversely, display device 120-1 can receive information from cloud network 190 and/or computer system 170 and communicate some or all of the received information to sensor control device 102, medication delivery device 152, or both. Computer system 170 may be a personal computer, a server terminal, a laptop computer, a tablet, or other suitable data processing device. Computer system 170 can include or present software for data management and analysis and communication with the components in system 100. Computer system 170 can be used by the user or a medical professional to display and/or analyze analyte data measured by sensor control device 102. Furthermore, although FIG. 1B depicts a single sensor control device 102, a single medication delivery device 152, and two display devices 120-1 and 120-2, those of skill in the art will appreciate that system 100 can include a plurality of any of the aforementioned devices, wherein each plurality of devices can comprise the same or different types of devices.

Referring still to FIG. 1B, according to some embodiments, trusted computer system 180 can be within the possession of a manufacturer or distributor of a component of system 100, either physically or virtually through a secured connection, and can be used to perform authentication of the devices of system 100 (e.g., devices 102, 120-n, 152), for secure storage of the user's data, and/or as a server that serves a data analytics program (e.g., accessible via a web browser) for performing analysis on the user's measured analyte data and medication history. Trusted computer system 180 can also act as a data hub for routing and exchanging data between all devices in communication with system 180 through cloud network 190. In other words, all devices of system 100 that are capable of communicating with cloud network 190 (e.g., either directly with an internet connection or indirectly via another device), are also capable of communicating with all of the other devices of system 100 that are capable of communicating with cloud network 190, either directly or indirectly.

Display device 120-2 is depicted in communication with cloud network 190. In this example, device 120-2 can be in the possession of another user that is granted access to the analyte and medication data of the person wearing sensor control device 102. For example, the person in possession of display device 120-2 can be a parent of a child wearing sensor control device 102, as one example, or a caregiver of an elderly patient wearing sensor control device 102, as another example. System 100 can be configured to communicate analyte and medication data about the wearer through cloud network 190 (e.g., via trusted computer system 180) to another user with granted access to the data.

The analyte monitoring functionality of dose guidance system 100 can be realized through inclusion of one or more devices capable of collecting, processing, and displaying analyte data of the user. Example embodiments of such devices and their methods of use are described in International Publ. No. WO 2018/152241 and U.S. Patent Publ. No. 2011/0213225, both of which are incorporated by reference herein in their entireties for all purposes.

Analyte monitoring can be performed in numerous different ways. “Continuous Analyte Monitoring” devices (e.g., “Continuous Glucose Monitoring” devices), for example, can transmit data from a sensor control device to a display device continuously or repeatedly with or without prompting, e.g., automatically according to a schedule. “Flash Analyte Monitoring” devices (e.g., “Flash Glucose Monitoring” devices or simply “Flash” devices), as another example, can transfer data from a sensor control device in response to a user-initiated request for data by a display device (e.g., a scan), such as with a Near Field Communication (NFC) or Radio Frequency Identification (RFID) protocol.

Analyte monitoring devices that utilize a sensor configured to be placed partially or wholly within a user's body can be referred to as in vivo analyte monitoring devices or in vivo glucose monitoring devices. For example, an in vivo sensor can be placed in the user's body such that at least a portion of the sensor is in contact with a bodily fluid (e.g., interstitial (ISF) fluid such as dermal fluid in the dermal layer or subcutaneous fluid beneath the dermal layer, blood, or others) and can measure an analyte concentration in that bodily fluid. In vivo sensors can use various types of sensing techniques (e.g., chemical, electrochemical, or optical). Some systems utilizing in vivo analyte sensors can also operate without the need for finger stick calibration.

“In vitro” devices are those where a sensor is brought into contact with a biological sample outside of the body (or rather “ex vivo”). These devices typically include a port for receiving an analyte test strip carrying bodily fluid of the user, which can be analyzed to determine the user's blood glucose level. Other ex vivo devices have been proposed that attempt to measure the user's internal analyte level non-invasively, such as by using an optical technique that can measure an internal body analyte level without mechanically penetrating the user's body or skin. In vivo and ex vivo devices often include in vitro capability (e.g., an in vivo display device that also includes a test strip port).

The present subject matter will be described primarily with respect to sensors capable of measuring a glucose concentration, although detection and measurement of concentrations of other analytes are within the scope of the present disclosure. These other analytes can include, ketones, lactate, or alcohol. Further, additional analytes may include oxygen, hemoglobin A1C, acetyl choline, amylase, bilirubin, cholesterol, chorionic gonadotropin, creatine kinase (e.g., CK-MB), creatine, DNA, fructosamine, glutamine, growth hormones, hormones, peroxide, prostate-specific antigen, prothrombin, RNA, thyroid stimulating hormone, troponin and others. The concentration of drugs, such as, for example, antibiotics (e.g., gentamicin, vancomycin, and the like), digitoxin, digoxin, drugs of abuse, theophylline, and warfarin, may also be monitored. The sensor can be configured to measure two or more different analytes at the same or different times. The sensor control device can be coupled with two or more sensors, where one sensor is configured to measure a first analyte (e.g., glucose) and the other one or more sensors are configured to measure one or more different analytes (e.g., any of those described herein). In other embodiments, a user can wear two or more sensor control devices, each of which is capable of measuring a different analyte.

The embodiments described herein can be used with all types of in vivo, in vitro, and ex vivo devices capable of monitoring the aforementioned analytes and others.

In many embodiments, the sensor operation can be controlled by sensor control device 102. The sensor can be mechanically and communicatively coupled with sensor control device 102, or can be just communicatively coupled with sensor control device 102 using a wireless communication technique. Sensor control device 102 can include the electronics and power supply that enable and control analyte sensing performed by the sensor. The sensor or sensor control device 102 can be self-powered such that a battery is not required. Sensor control device 102 can also include communication circuitry for communicating with another device that may or may not be local to the user's body (e.g., a display device). Sensor control device 102 can reside on the body of the user (e.g., attached to or otherwise placed on the user's skin, or carried in the user's clothes, etc.). Sensor control device 102 can also be implanted within the body of the user along with the sensor. Functionality of sensor control device 102 can be divided between a first component implanted within the body (e.g., a component that controls the sensor) and a second component that resides on or otherwise outside the body (e.g., a relay component that communicates with the first component and also with an external device like a computer or smartphone). In other embodiments, sensor control device 102 can be external to the body and configured to non-invasively measure the user's analyte levels. The sensor control device, depending on the actual implementation or embodiment, can also be referred to as a “sensor control unit,” an “on-body electronics” device or unit, an “on-body” device or unit, an “in body electronics” device or unit, an “in-body” device or unit, or a “sensor data communication” device or unit, to name a few.

Sensor control device 102 may include a user interface (e.g., a touchscreen) and be capable of processing the analyte data and displaying the resultant calculated analyte levels to the user. In such cases, the dose guidance embodiments described herein can be implemented directly by sensor control device 102, in whole or in part. In many embodiments, the physical form factor of sensor control device 102 is minimized (e.g., to minimize the appearance on the user's body) or sensor control device 102 may be inaccessible to the user (e.g., if wholly implanted), or other factors may make it desirable to have a display device usable by the user to read analyte levels and interface with the sensor control device.

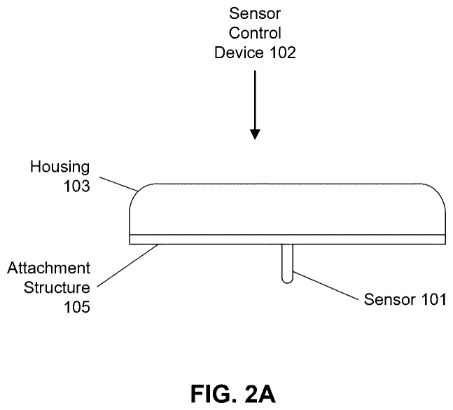

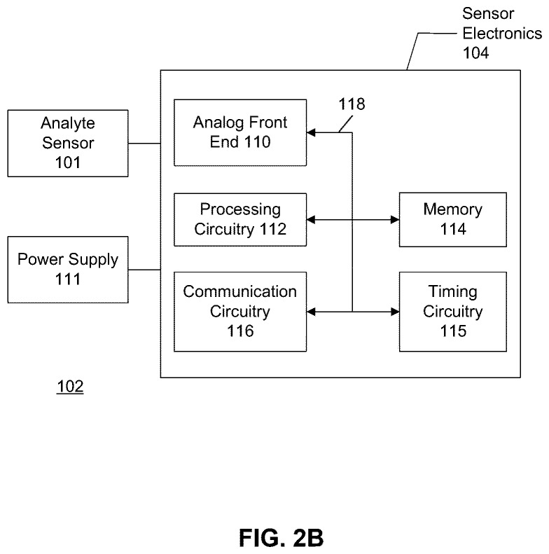

FIG. 2A is a side view of an example embodiment of sensor control device 102. Sensor control device 102 may be referred to as an analyte monitoring device, or as a glucose monitoring device where the analyte to be monitored is glucose (e.g., a continuous glucose monitor (CGM)). Sensor control device 102 may include a glucose sensor and sensor electronics. The glucose sensor may include a first portion configured to be positioned under a skin surface for detecting glucose in a bodily fluid, such as interstitial fluid, and a second portion configured to be positioned above the skin surface. Sensor electronics (as further described herein) are permanently or removably coupled to the second portion of the glucose sensor. Sensor electronics may be configured to transmit glucose data to other devices, such as a display device, a remote server, or a medication delivery device, among others.

Sensor control device 102 can include a housing or mount 103 for sensor electronics (FIG. 2B), which can be electrically coupled with an analyte sensor 101, which is configured here as an electrochemical sensor. According to some embodiments, sensor 101 can be configured to reside partially within a user's body (e.g., through an exterior-most surface of the skin) where it can make fluid contact with a user's bodily fluid and be used, along with the sensor electronics, to measure analyte-related data of the user. A structure for attachment 105, such as an adhesive patch, can be used to secure housing 103 to a user's skin. Sensor 101 can extend through attachment structure 105 and project away from housing 103. Those of skill in the art will appreciate that other forms of attachment to the body and/or housing 103 may be used, in addition to or instead of adhesive, and are fully within the scope of the present disclosure.

Sensor control device 102 can be applied to the body in any desired manner. For example, an insertion device (not shown), sometimes referred to as an applicator, can be used to position all or a portion of analyte sensor 101 through an external surface of the user's skin and into contact with the user's bodily fluid. In doing so, the insertion device can also position sensor control device 102 onto the skin. In other embodiments, the insertion device can position sensor 101 first, and then accompanying electronics (e.g., wireless transmission circuitry and/or data processing circuitry, and the like) can be coupled with sensor 101 afterwards (e.g., inserted into a mount), either manually or with the aid of a mechanical device. Examples of insertion devices are described in U.S. Patent Publication Nos. 2008/0009692, 2011/0319729, 2015/0018639, 2015/0025345, and 2015/0173661, 2018/0235520, all which are incorporated by reference herein in their entireties for all purposes.

FIG. 2B is a block diagram depicting an example embodiment of sensor control device 102 having analyte sensor 101 and sensor electronics 104. Sensor electronics 104 can be implemented in one or more semiconductor chips (e.g., an application specific integrated circuit (ASIC), processor or controller, memory, programmable gate array, and others). In the embodiment of FIG. 2B, sensor electronics 104 includes high-level functional units, including an analog front end (AFE) 110 configured to interface in an analog manner with sensor 101 and convert analog signals to and/or from digital form (e.g., with an A/D converter), a power supply 111 configured to supply power to the components of sensor control device 102, processing circuitry 112, memory 114, timing circuitry 115 (e.g., such as an oscillator and phase locked loop for providing a clock or other timing to components of sensor control device 102), and communication circuitry 116 configured to communicate in wired and/or wireless fashion with one or more devices external to sensor control device 102, such as display device 120 and/or medication delivery device 152.

Sensor control device 102 can be implemented in a highly interconnected fashion, where power supply 111 is coupled with each component shown in FIG. 2B and where those components that communicate or receive data, information, or commands (e.g., AFE 110, processing circuitry 112, memory 114, timing circuitry 115, and communication circuitry 116), can be communicatively coupled with every other such component over, for example, one or more communication connections or buses 118.

Processing circuitry 112 can include one or more processors, microprocessors, controllers, and/or microcontrollers, each of which can be a discrete chip or distributed amongst (and a portion of) a number of different chips. Processing circuitry 112 can include on-board memory. Processing circuitry 112 can interface with communication circuitry 116 and perform analog-to-digital conversions, encoding and decoding, digital signal processing and other functions that facilitate the conversion of data signals into a format (e.g., in-phase and quadrature) suitable for wireless or wired transmission. Processing circuitry 112 can also interface with communication circuitry 116 to perform the reverse functions necessary to receive a wireless transmission and convert it into digital data or information.

Processing circuitry 112 can execute instructions stored in memory 114. These instructions can cause processing circuitry 112 to process raw analyte data (or pre-processed analyte data) and arrive at a final calculated analyte level. Instructions stored in memory 114, when executed, can cause processing circuitry 112 to process raw analyte data to determine one or more of: a calculated analyte level, an average calculated analyte level within a predetermined time window, a calculated rate-of-change of an analyte level within a predetermined time window, and/or whether a calculated analyte metric exceeds a predetermined threshold condition. These instructions can also cause processing circuitry 112 to read and act on received transmissions, to adjust the timing of timing circuitry 115, to process data or information received from other devices (e.g., calibration information, encryption or authentication information received from display device 120, and others), to perform tasks to establish and maintain communication with display device 120, to interpret voice commands from a user, to cause communication circuitry 116 to transmit, and others. In embodiments where sensor control device 102 includes a user interface, then the instructions can cause processing circuitry 112 to control the user interface, read user input from the user interface, cause the display of information on the user interface, format data for display, and others. The functions described here that are coded in the instructions can instead be implemented by sensor control device 102 with the use of a hardware or firmware design that does not rely on the execution of stored software instructions to accomplish the functions.

Memory 114 can be shared by one or more of the various functional units present within sensor control device 102, or can be distributed amongst two or more of them (e.g., as separate memories present within different chips). Memory 114 can also be a separate chip of its own. Memory 114 is non-transitory, and can be volatile (e.g., RAM, etc.) and/or non-volatile memory (e.g., ROM, flash memory, F-RAM, etc.).

Communication circuitry 116 can be implemented as one or more components (e.g., transmitter, receiver, transceiver, passive circuit, encoder, decoder, and/or other communication circuitry) that perform the functions for communications over the respective communications paths or links. Communication circuitry 116 can include or be coupled to one or more antenna for wireless communication.

Power supply 111 can include one or more batteries, which can be rechargeable or single-use disposable batteries. Power management circuitry can also be included to regulate battery charging and monitor usage of power supply 111, boost power, perform DC conversions, and the like.

Additionally, an on-skin or sensor temperature reading or measurement can be collected by an optional temperature sensor. Those readings or measurements can be communicated (either individually or as an aggregated measurement over time) from sensor control device 102 to another device (e.g., display device 120). The temperature reading or measurement, however, can be used in conjunction with a software routine executed by sensor control device 102 or display device 120 to correct or compensate the analyte measurement output to the user, instead of or in addition to, actually outputting the temperature measurement to the user.

Display device 120 can be configured to display information pertaining to system 100 to the user and accept or receive input from the user also pertaining to system 100. Display device 120 can display recent measured analyte levels, in any number of forms, to the user. The display device can display historical analyte levels of the user as well as other metrics that describe the user's analyte information (e.g., time in range, ambulatory glucose profile (AGP), hypoglycemia risk levels, etc.). Display device 120 can display medication delivery information, such as historical dose information and the times and dates of administration, dose guidance system settings or parameters, or dose recommendations, among other information. Display device 120 can display alarms, alerts, or other notifications pertaining to analyte levels and/or medication delivery.

Display device 120 can be dedicated for use with system 100 (e.g., an electronic device designed and manufactured for the primary purpose of interfacing with an analyte sensor and/or a medication delivery device), as well as devices that are multifunctional, general purpose computing devices such as a handheld or portable mobile communication device (e.g., a smartphone or tablet), or a laptop, personal computer, or other computing device. Display device 120 can be configured as a mobile smart wearable electronics assembly, such as a smart glass or smart glasses, or a smart watch or wristband. Display devices, and variations thereof, can be referred to as “reader devices,” “readers,” “handheld electronics” (or handhelds), “portable data processing” devices or units, “information receivers,” “receiver” devices or units (or simply receivers), “relay” devices or units, or “remote” devices or units, to name a few.

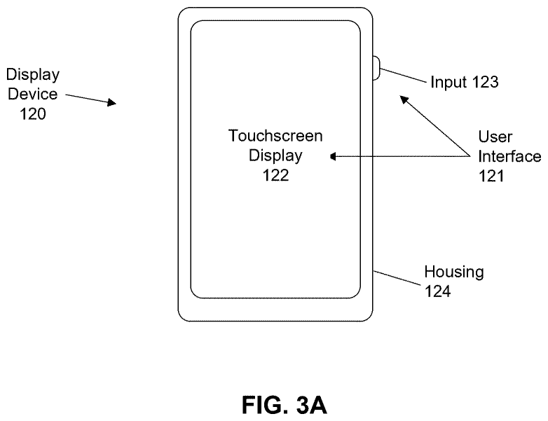

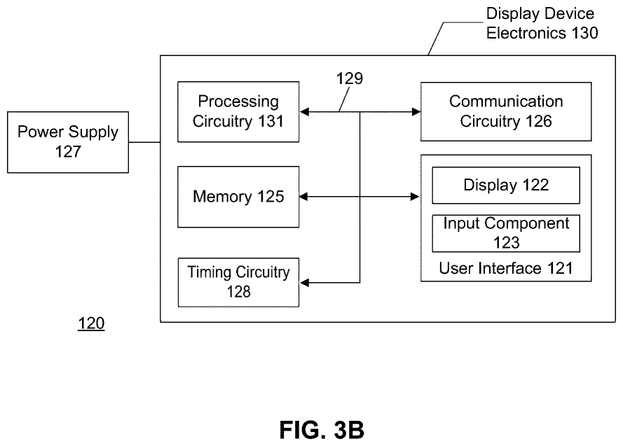

FIG. 3A is a schematic view depicting an example embodiment of display device 120. Here, display device 120 includes a user interface 121 and a housing 124 in which display device electronics 130 (FIG. 3B) are held. User interface 121 can be implemented as a single component (e.g., a touchscreen capable of input and output) or multiple components (e.g., a display and one or more devices configured to receive user input). In this embodiment, user interface 121 includes a touchscreen display 122 (configured to display information and graphics and accept user input by touch) and an input button 123, both of which are coupled with housing 124.

Display device 120 can have software stored thereon (e.g., by the manufacturer or downloaded by the user in the form of one or more “apps” or other software packages) that interface with sensor control device 102, medication delivery device 152, and/or the user. In addition, or alternatively, the user interface can be affected by a web page displayed on a browser or other internet interfacing software executable on display device 120.

FIG. 3B is a block diagram of an example embodiment of a display device 120 with display device electronics 130. Here, display device 120 includes user interface 121 including display 122 and an input component 123 (e.g., a button, actuator, touch sensitive switch, capacitive switch, pressure sensitive switch, jog wheel, microphone, speaker, or the like), processing circuitry 131, memory 125, communication circuitry 126 configured to communicate to and/or from one or more other devices external to display device 120), a power supply 127, and timing circuitry 128 (e.g., such as an oscillator and phase locked loop for providing a clock or other timing to components of sensor control device 102). Each of the components can be implemented as one or more different devices or can be combined into a multifunctional device (e.g., integration of processing circuitry 131, memory 125, and communication circuitry 126 on a single semiconductor chip). Display device 120 can be implemented in a highly interconnected fashion, where power supply 127 is coupled with each component shown in FIG. 3B and where those components that communicate or receive data, information, or commands (e.g., user interface 121, processing circuitry 131, memory 125, communication circuitry 126, and timing circuitry 128), can be communicatively coupled with every other such component over, for example, one or more communication connections or buses 129. FIG. 3B is an abbreviated representation of the typical hardware and functionality that resides within a display device and those of ordinary skill in the art will readily recognize that other hardware and functionality (e.g., codecs, drivers, glue logic) can also be included.

Processing circuitry 131 can include one or more processors, microprocessors, controllers, and/or microcontrollers, each of which can be a discrete chip or distributed amongst (and a portion of) a number of different chips. Processing circuitry 131 can include on-board memory. Processing circuitry 131 can interface with communication circuitry 126 and perform analog-to-digital conversions, encoding and decoding, digital signal processing and other functions that facilitate the conversion of data signals into a format (e.g., in-phase and quadrature) suitable for wireless or wired transmission. Processing circuitry 131 can also interface with communication circuitry 126 to perform the reverse functions necessary to receive a wireless transmission and convert it into digital data or information.

Processing circuitry 131 can execute software instructions stored in memory 125. These instructions can cause processing circuitry 131 to process raw analyte data (or pre-processed analyte data) and arrive at a corresponding analyte level suitable for display to the user. These instructions can cause processing circuitry 131 to read, process, and/or store a dose instruction from the user, and because the dose instruction to be communicated to medication delivery device 152. These instructions can cause processing circuitry 131 to execute user interface software adapted to present an interactive group of graphical user interface screens to the user for the purposes of configuring system parameters (e.g., alarm thresholds, notification settings, display preferences, and the like), presenting current and historical analyte level information to the user, presenting current and historical medication delivery information to the user, collecting other non-analyte information from the user (e.g., information about meals consumed, activities performed, medication administered, and the like), and presenting notifications and alarms to the user. These instructions can also cause processing circuitry 131 to cause communication circuitry 126 to transmit, can cause processing circuitry 131 to read and act on received transmissions, to read input from user interface 121 (e.g., entry of a medication dose to be administered or confirmation of a recommended medication dose), to display data or information on user interface 121, to adjust the timing of timing circuitry 128, to process data or information received from other devices (e.g., analyte data, calibration information, encryption or authentication information received from sensor control device 102, and others), to perform tasks to establish and maintain communication with sensor control device 102, to interpret voice commands from a user, and others. The functions described here that are coded in the instructions can instead be implemented by display device 120 with the use of a hardware or firmware design that does not rely on the execution of stored software instructions to accomplish the functions.

Memory 125 can be shared by one or more of the various functional units present within display device 120, or can be distributed amongst two or more of them (e.g., as separate memories present within different chips). Memory 125 can also be a separate chip of its own. Memory 125 is non-transitory, and can be volatile (e.g., RAM, etc.) and/or non-volatile memory (e.g., ROM, flash memory, F-RAM, etc.).

Communication circuitry 126 can be implemented as one or more components (e.g., transmitter, receiver, transceiver, passive circuit, encoder, decoder, and/or other communication circuitry) that perform the functions for communications over the respective communications paths or links. Communication circuitry 126 can include or be coupled to one or more antenna for wireless communication.

Power supply 127 can include one or more batteries, which can be rechargeable or single-use disposable batteries. Power management circuitry can also be included to regulate battery charging and monitor usage of power supply 127, boost power, perform DC conversions, and the like.

Display device 120 can also include one or more data communication ports (not shown) for wired data communication with external devices such as computer system 170, sensor control device 102, or medication delivery device 152. Display device 120 may also include an integrated or attachable in vitro glucose meter, including an in vitro test strip port (not shown) to receive an in vitro glucose test strip for performing in vitro blood glucose measurements.

Display device 120 can display the measured analyte data received from sensor control device 102 and can also be configured to output alarms, alert notifications, glucose values, etc., which may be visual, audible, tactile, or any combination thereof. Sensor control device 102 and/or medication delivery device 152 can also be configured to output alarms, or alert notifications in visible, audible, tactile forms or combination thereof. Further details and other display embodiments can be found in, e.g., U.S. Patent Publ. No. 2011/0193704, which is incorporated herein by reference in its entirety for all purposes.

Medication delivery device 152 may include an infusion pump, a patch pump, or an injection pen, among other devices for administering a medication. Dose guidance system 100 may include automatic dose capture devices, such as a smart pen cap configured to be disposed on an injection pen to collect medication information, such as the type of medication, time of administration of a dose, or dose amount, among other information.

Dose guidance system 100 may determine a dose of medication to administer and may display the recommended dose on display device 120. The user may then use a medication delivery device 152 to administer the recommended dose. The medication delivery device 152 or dose capture device may communicate collected dose information to the dose guidance system 100. Dose guidance system may communicate the recommended dose to medication delivery device 152. Medication delivery device 152 may display the recommended dose. Medication delivery device 152 may automatically administer the received dose. Medication delivery device 152 may require user confirmation to administer the received dose.

Dose guidance system 100 may use a glucose pattern analysis to analyze glucose data received by a glucose monitoring device. The glucose pattern analysis may be based on quantitative assessment of the user's analyte data during one or more time of day (TOD) periods. Analyte data collected over a number of days can be assessed to determine one or more metrics that are descriptive of the relevant glycemic risk in the corresponding TOD period. Glycemic risks and glucose patterns may be determined based on various methods, as will be appreciated by one skilled in the art. U.S. Publication No. 2014/0188400A1, incorporated herein by reference in its entirety, provides an example implement for determining and deriving glycemic risk metrics that can be used in a glucose pattern analysis. These metrics can be used to classify the analyte data from the TOD period as one of multiple patterns. The patterns can be indicative of a common or prevalent glucose behavior or trend for that TOD. The dose guidance system may be configured to categorize the glucose data for each TOD into a glucose pattern of a predetermined list of glucose patterns. The dose guidance system may identify one or more of three glucose patterns: a Low pattern, a High/Low pattern, and a High pattern. However, other embodiments may use fewer patterns or may include additional patterns. For example, the dose guidance system may include only a Low pattern and a High pattern. Exemplary glucose pattern analyses are described in U.S. Publication No. 2021/0050085A1 and U.S. Publication No. 2022/0249779A1, which applications are incorporated herein by reference in their entireties.

The glucose data may be divided into one or more TOD periods. The TOD periods may include a breakfast (or post-breakfast period), a lunch (or post-lunch period), a dinner (or post-dinner) period, and an overnight period. However, additional or fewer TOD periods may be used. For example, the overnight period may be split into first and second overnight periods. Alternatively, the mealtime periods may include fewer than three mealtime periods, such as for users who do not take a medication dose at each meal the corresponding meal period may be excluded. The remaining TOD periods may be adjusted accordingly.

The dose guidance system may determine a pattern for each TOD period to determine a pattern assessment for that period. The dose guidance system may determine a central tendency value, and a variability value for the user's glucose data for each TOD period. The user's analyte data may be available from the user's own records or those of the user's healthcare professional, or the user's analyte data may have been collected by the dose guidance system, for example. The analyte data preferably spans a multi-day period (e.g., two days, two weeks, one month, etc.) such that sufficient data exists within the TOD period to make a reliable determination. In other embodiments, the method can be performed in real-time based on limited data. The dose guidance system can use any type of central tendency metric that correlates to a central tendency of the data including, but not limited to, a median or mean value. Any desired variability metric can also be used including, but not limited to, variability ranges that span the entire data set (e.g., from the minimal value to the maximum value), variability ranges that span a majority of the data but less than the entire data set so as to lessen the significance of outliers (e.g., from the 90th percentile to the 10th percentile, from the 75th percentile to the 25th percentile), or variability ranges that target a specific asymmetrical range (e.g., low range variability, which can span a range, e.g., from or in proximity with the central tendency value to a lower value of data, e.g., the 25th percentile, the 10th percentile, or the minimal value). The selection of the metrics to represent the central tendency and variability can vary based on the implementation.

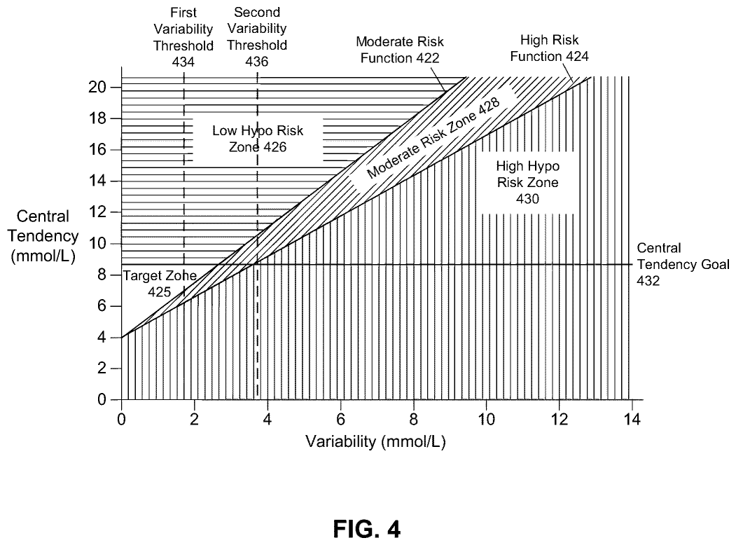

The dose guidance system can assess a risk of hypoglycemia for each TOD period based on one or more of a glucose central tendency value, a glucose variability value, and a hypoglycemia risk metric for the corresponding TOD period. The glucose central tendency value and glucose variability value may correspond to a hypoglycemia risk level. For example, the relationship between glucose central tendency, variability, and hypoglycemia risk is shown graphically on the central tendency-variability plot of FIG. 4. The plot 400 includes two or more zones. The zones are divided by hypoglycemia risk curves. The hypoglycemia risk curves may correspond to a constant value for a hypoglycemia risk metric. In FIG. 4, two hypoglycemia risk curves 422, 424 are shown. A first hypoglycemia risk curve 424 may divide the plot 400 into a high-risk zone 430 and a moderate-risk zone 428. Second hypoglycemia risk curve 422 may divide the plot 400 into a low-risk zone 426 and the moderate-risk zone 428. A target glucose level 432 may also be plotted. The target glucose level 432 may be a glucose median, or other measure of central tendency of glucose values, such as an average glucose. A target zone 425 may include a region bounded by the second hypoglycemia risk curve 422 and the target glucose level 432. Plot 400 may further one or more glucose variability thresholds. Plot 400 shows a first variability threshold 434 and a second variability threshold 436, which may be used for example to assess the glucose variability as low, moderate or high.

The dose guidance system can assess a hyperglycemia risk metric based on the central tendency value. The hyperglycemia risk can be evaluated by comparison of the central tendency value for the particular TOD period to a central tendency goal or threshold. The magnitude and/or sign of the difference of the central tendency value from the goal 432 can identify the amount of risk. For example, a low risk can be present if the central tendency value is less than the goal (e.g., a negative value). A moderate risk can be present if the central tendency value exceeds the goal (e.g., a positive value) by less than a threshold amount (e.g., 5 percent, 10 percent, etc.). A high risk can be present if the central tendency value exceeds the goal by a value greater than the threshold amount. The use of three discrete groupings for hyperglycemia risk (e.g., low, moderate, high) is an example and any number of two or more groupings can be used.

Other metrics such as glucose variability risk can also be assessed. For example, a variability value less than a first variability threshold can be indicative of a low variability risk, a variability value greater than the first variability threshold and less than a second variability threshold can be indicative of a moderate variability risk, and a variability value greater than the second variability threshold can be indicative of a high variability risk. Again, the use of three discrete groupings for variability risk is an example.

The dose guidance system can determine a glucose pattern type for each TOD period based on the assessed one or more risk metrics. In one example embodiment, pattern determination can be assessed with the hypoglycemia and hyperglycemia risk metrics. If the hypoglycemia risk metric is high, then the pattern can be set as a “Low” pattern. Otherwise, if the hypoglycemia risk is moderate and the hyperglycemia risk is either high or moderate then the pattern can be set as a “High/Low” (or moderate) pattern. Otherwise, if the hyperglycemia risk is high or moderate and the hypoglycemia risk is low, then the pattern can be set as a “High” pattern. If both the hyperglycemia risk and hypoglycemia risk are low, then the pattern identified can be No Problem (e.g., an “OK” message is displayed or outputted).

The number of pattern types in the pattern types themselves can vary from those described in this embodiment (e.g., Low, High/Low, High). Once a pattern type for the TOD period has been determined, the dose guidance system can store an indicator of the pattern type in a memory location for use in determining a titration recommendation. The dose guidance system can then determine a titration recommendation once completing the glucose pattern analysis for each relevant TOD period.

Once the patterns for each TOD period are determined, the dose guidance system evaluates whether the pattern type for an overnight TOD period is low. If the pattern is Low, the dose guidance system generates a recommendation to reduce all relevant doses, including at least a basal dose and optionally, one or more of a meal dose, pre-meal correction dose, or post-prandial dose, by an equal amount, for example, 10%. Titration recommendation rules for the Low patterns can include, for overnight TOD periods, generating a recommendation to reduce the long-acting insulin dose(s) or basal rate. If any other TOD period has a Low pattern, the dose guidance system can generate a recommendation to reduce the fixed meal dose for the relevant TOD period only.

If there is at least one Low pattern, then no titration guidance for any High pattern TOD period is provided. The idea here is to emphasize prevention of hypoglycemia and to only increase doses when the risk of hypoglycemia is low in all TOD periods. Also, it is possible that in some situations, when a TOD period has a High pattern, this could be caused by a prior TOD period having a Low pattern, and the patient is overeating to compensate—so addressing the Low pattern can in itself help address a subsequent High pattern. If the pattern is not High, the process waits or terminates without generating a recommendation or passes to a High pattern evaluation.

Accordingly, for High/Low patterns, the dose guidance system generates no titration guidance. If there are no TOD periods where titration guidance can be given and data is sufficient for all time periods, then the dose guidance system can provide a message to the patient indicating that they need to address glucose variability before further titration guidance can be given. Also, the dose guidance system can provide a report to the patient's HCP to consider alternative medications or therapies that can address glucose variability.

Dose guidance system may generate titration recommendations for High patterns when there are no Low pattern TOD periods. If the overnight period has a High pattern and there is no other period with moderate risk of hypoglycemia, the dose guidance system can increase the long-acting insulin dose(s) or basal rate recommendation. If the overnight period has a High pattern, and there is at least one other non-dinner period with moderate risk of hypoglycemia, then the dose guidance system can decrease the meal insulin dose associated with any period with moderate risk of hypoglycemia. If the overnight TOD period has no moderate risk of hypoglycemia and no High pattern, then the dose guidance system can generate a recommendation to increase the meal insulin dose associated with the first TOD period with a High pattern. If the overnight period has a moderate risk of hypoglycemia, and the only post-meal period with a High pattern is dinner, the dose guidance system can generate a recommendation to increase the long-acting insulin dose(s) or basal rate. If the overnight period has a moderate risk of hypoglycemia, but not the post-dinner period, the dose guidance system can generate a recommendation to increase the meal insulin dose associated with the first TOD period with a High pattern.

In alternative embodiments, the pre-meal glucose can be higher or lower than a target glucose (for example, 120 mg/dL). The glucose data for each meal that contributes to the calculation of the hypoglycemia and hyperglycemia risk metrics can be modified to compensate for the effects from a prior meal or condition that affects glucose that is not due to the current meal. The dose guidance system can modify these data by subtracting the offset so the resulting starting glucose is the target level. Alternatively, the dose guidance system can modify these data by a “triangle” function where, for the meal start time, the difference between the meal start glucose and the target glucose is subtracted, but this modification is reduced over time; either linearly for a defined period (e.g., three (3) hours), or another decay function. Alternatively, this function can itself be a function of meal start glucose level or glucose trend, and/or when the previous meal dose was taken.

Some embodiments described herein rely on analysis of TOD periods, such as to titrate medication doses. A day or 24-hour period may be divided into multiple TOD periods relevant to a patient. For example, TOD periods may be based on mealtimes. Medication doses taken during a day can be associated with a particular TOD period. For example, there may be four TOD periods corresponding to breakfast, lunch, dinner, and overnight (e.g., fasting) periods. Glucose data collected throughout the day, such as by a glucose monitoring device, may be segmented into a corresponding TOD period. The glucose data for the corresponding TOD period may be analyzed to determine how and whether to adjust an associated medication dose for that TOD period. Glucose patterns (e.g., High, High/Low, Low, or No pattern), or glycemic risk metrics for a risk of hyperglycemia and hypoglycemia, may be determined for each TOD period based on the glucose data as discussed herein, and the glucose patterns or glycemic risk metrics may be used to titrate each medication dose.

TOD periods may be predetermined by the dose guidance system. For example, dose guidance system may set TOD periods corresponding to typical mealtimes, such as a breakfast period from 8 AM-12 PM, a lunch period from 12 PM-6 PM, a dinner period from 6 PM-12 AM, and an overnight period from 12 AM-8 AM. It is understood that other mealtimes may be used. For example, the TOD periods may include a breakfast period from 7 AM-11 AM, a lunch period from 11 AM-5 PM, a dinner period from 5 PM-11 PM, and an overnight period from 11 PM-7 AM. The TOD periods may each include the same number of hours, or may include different numbers of hours. The TOD periods may be fixed and do not change. Alternatively, the predetermined TOD periods may be used as a default, and the dose guidance system may adjust the TOD periods over time, and may adjust the TOD periods automatically based on collected data and/or based on user input.

In some embodiments, TOD periods can be set based on user input. The dose guidance system may prompt the user to enter the user's typical mealtimes, such as to enter a breakfast time, lunch time, and dinner time via an input of a display device. The dose guidance system can then use TOD periods for titrating the medication doses based on the user's entered mealtimes. For example, a display device, such as a smartphone, may include a user interface that allows the user to select typical mealtimes. The user may select mealtimes from a predetermined list of times. For example, the user may enter 8 AM, 12 PM, and 6 PM for breakfast, lunch and dinner, respectively, such that the TOD periods used by the dose guidance system are 8 AM-12 PM, 12 PM-6 PM, 6 PM-12 AM, and 12 AM-8 AM. In such embodiments, the overnight period may be based on the TOD period being a fixed amount of time, e.g., 6 hours, such that the overnight period is the 6 hours preceding the breakfast time. Alternatively, the overnight period may be assumed to start at midnight and end at the breakfast time.

The use of fixed TOD periods for titrating medication doses may be ineffective where the patient's mealtimes (and corresponding medication dose times) deviate from the fixed TOD periods, whether predetermined by the system or input by the user at set-up of the dose guidance system. Often, a user's mealtimes may vary between days or weeks. For example, the patient may shift to a different work schedule, such as a shift worker who begins working nightshift. Other patients may frequently travel, and may be in different time zones which may impact their mealtimes. Other patients may have atypical or varying mealtimes for other reasons. Accordingly, the use of predetermined TOD periods may result in TOD periods not reflective of the user's schedule. For example, if the TOD periods are set to 8 AM-12 PM, 12 PM-6 PM, 6 PM-12 AM for breakfast, lunch, and dinner, respectively, and the user's breakfast, lunch, and dinner times change to 1 PM, 5 PM, 11 PM, the system will incorrectly identify the breakfast segments as lunch segments, lunch segments as dinner segments, and so on. The resulting mismatch between the TOD periods used for titration and the patient's actual mealtimes (and medication dose times) may negatively impact the titration process. In order to address this problem, the TOD periods or mealtimes used to set the TOD day periods may be determined based on user data.

In some embodiments, TOD periods may be determined based on the time doses are administered. The system may receive dose times from the medication delivery device, or a dose monitoring device (e.g., a smart pen cap). The user may manually enter doses administered into the dose guidance system, such as via an input of a display device. The system may determine the TOD periods for titration based on the time doses are actually administered by the user. The system may use one or more days of data, and may average the dose times over multiple days to determine dose times.

In some embodiments, TOD periods may be determined using a model, such as a machine learning model. The model can be provided with glucose data and insulin data, such as dose information, including for example medication types (e.g., insulin types), dose types, dose times, or dose amounts, among other information, such as meal information. Machine learning models can include, by way of example and not limitation, models trained using or encompassing decision tree analysis, gradient boosting, adaptive boosting, artificial neural networks or variants thereof, linear discriminant analysis, nearest neighbor analysis, support vector machines, supervised or unsupervised classification, and others. The models can also include algorithmic or rules-based models in addition to machine learned models. Machine learning involves computers discovering how they can perform tasks without being explicitly programmed to do so. Machine learning includes, but is not limited to, artificial intelligence, deep learning, fuzzy learning, supervised learning, and unsupervised learning, etc.

Machine learning algorithms may build an initial prediction model based on sample data, known as “training data”, in order to make predictions or decisions without being explicitly programmed to do so. This sample data may include glucose data and insulin data from the patient or from a population of patients. For supervised learning, the computer is presented with example inputs and their desired outputs and the goal is to learn a general rule that maps inputs to outputs. In another example, for unsupervised learning, no labels are given to the learning algorithm, leaving it on its own to find structure in its input. Unsupervised learning can be the ultimate goal such as discovering trends or patterns in data, or a means towards another goal, such as improved accuracy of future predictions.

A machine-learning engine may use various classifiers to map concepts or data to capture relationships between concepts (e.g., glucose data, insulin data, fasting periods, and TOD periods) and an accuracy of prior predicted patient outcomes. The classifier (discriminator) is trained to distinguish (recognize) in variations of data. In some aspects, machine learning models are trained on a remote machine learning platform using a history of glucose data and insulin data from the patient, or from a population of patients. In one embodiment, prediction models are continuously updated as new patient information is received.

Some embodiments described herein relate to systems and methods for detecting a change in the user's schedule. A change in the user's schedule can be determined by detecting a change in the user's fasting period. The longest interval between consecutive medication doses in a day is typically the interval between dinner and breakfast.

Accordingly, the system may determine a fasting period based on the longest gap between consecutive medication doses over a one day period (e.g., 24 hour period). For example, if the user's mealtimes are 8 AM, 12 PM, and 6 PM, and then the user's schedule changes such that mealtimes are 1 PM, 5 PM and 11 PM, the longest interval changes from 6 PM-8 AM to 11 PM-1 PM. If the system detects a change in the fasting period, the system determines a change in the user's schedule and can update the TOD periods accordingly, with the last medication dose before the fasting period identified as a dinner dose, and the first medication dose following the fasting period identified as a breakfast dose.

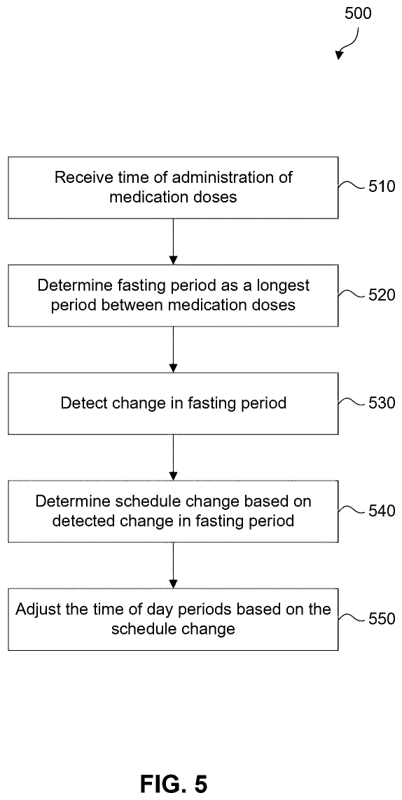

An exemplary embodiment for determining TOD periods is shown for example in FIG. 5. The dose guidance system receives the times at which medication doses are administered 510. The dose guidance system determines a fasting period based on the interval between consecutive doses that is the longest of the intervals between doses 520. The dose guidance system determines if there is a change in the fasting period 530. If a change in the fasting period is detected, the system determines that a schedule change has occurred based on the detected change in the fasting period 540. The system adjusts the TOD periods based on the determined change in schedule 550.

Alternatively, the fasting period (and the change in schedule) may be determined based on a measure of variability of the glucose data, referred to as a glucose variability. While fasting, the user's glucose variability is typically relatively low compared to other times of day when the user may be cating, exercising or performing other activities. The measure of glucose variability may be a standard deviation, coefficient of variability, difference between median and tenth percentile of glucose values (sometimes referred to as “South40”), or interquartile range, among other measures of variability. The glucose variability is assessed in a moving window of a predetermined number of hours. For example, the moving window may be 6 hours, and the variability is determined for each 6-hour period in a day, e.g., 12 PM-6 PM, 1 PM-7 PM, 2 PM-8 PM, etc. It should be understood that other periods for the moving window may be used, such as 4 hour periods, 9 hour periods, or 12 hour periods, among others. The fasting period may be determined to be the moving window with the lowest glucose variability. The fasting period may correspond to the longest period with low variability determined by summing the variability from the moving windows over a second predetermined number of hours.

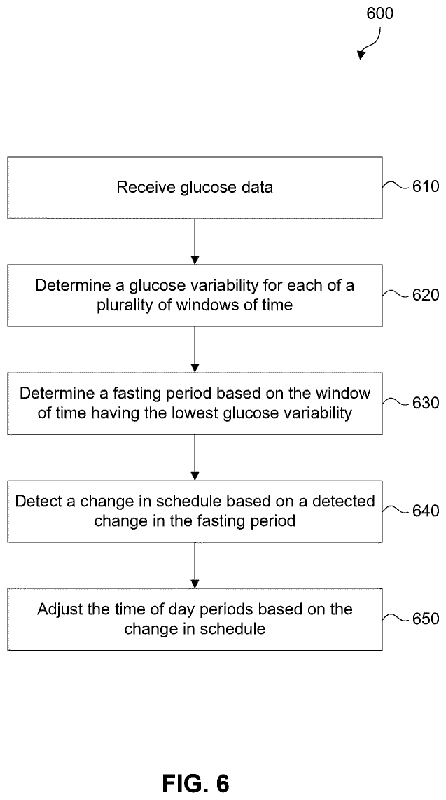

An exemplary embodiment of determining TOD periods for titration 600 is shown in FIG. 6. The dose guidance system receives glucose data over a predetermined period of time 610. A glucose variability is determined for each of a plurality of windows of time in a one day period 620. The system determines a fasting period based on the window of time having the lowest glucose variability 630. The system detects a change in schedule when there is a change in the fasting period 640. The system can adjust the TOD periods based on the detected change in schedule 650.

The dose guidance system may determine the user's schedule has changed based on a change in schedule for one day, or may require two or more days of data to determine and confirm that the user's schedule has actually changed. For example, the change in fasting period may be determined if a start time of the fasting period changes from a first time to a second time, and the fasting period starts at the second time for a minimum period of time, such as the fasting period starting at the second time for two or more days (or any other predetermined number of consecutive days). The system may determine a schedule change has taken place only if the shift in the fasting period is greater than a minimum threshold shift relative to the current or original fasting period. Requirements for additional days of data or for a minimum shift of the fasting period help to prevent false identification of adjusted TOD periods based on aberrations, such as adjusting TOD periods based on one or a few very late or very early mealtimes or meal-time doses. The system may output a notification or prompt for the user to confirm the schedule change upon detection of the schedule change. For example, display device may display a message asking the user if his or her schedule has changed, and the user may be prompted to input a confirmation in order for the system to begin using the changed schedule. The system may alternately update the TOD periods upon detection of a schedule change without notifying the user.

Alternatively, the fasting period may be detected by computing autocorrelation of continuous glucose data over one or more days. For purposes of detecting a fasting period by computing autocorrelation of continuous glucose data, one day may be defined as midnight of one day to midnight of the following day. Autocorrelation computes the correlation of a time-series data with a lagged version of the time-series data. Autocorrelation may be computed over a range of lag values, for example, starting from 15 minutes to 24 hours. The length of the fasting period may be the lower of the total of lag lengths corresponding to positive correlation and the total of lag lengths corresponding to negative correlation.

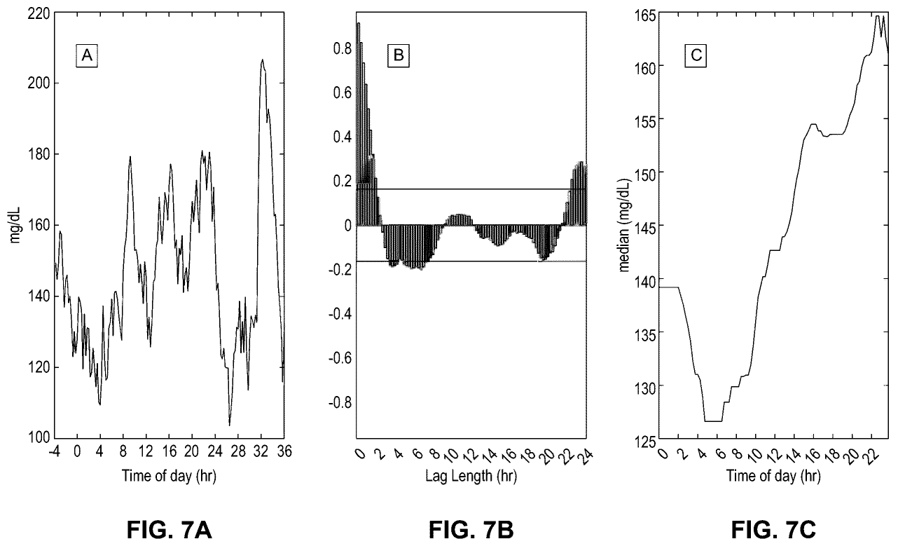

For example, FIG. 7A shows continuous glucose data collected over 36 hours. FIG. 7B shows autocorrelation of the glucose data, and has a positive correlation for 8 hours of lag lengths (i.e., 0-2.5 hours, 9-12 hours, and 21.5-24 hours), and has a negative correlation of 16 hours of lag lengths (i.e., 2.5-9 hours, 12-21.5 hours). The length of the fasting period is the lower of the two values, and is therefore 8 hours. FIG. 7C shows a glucose median calculated every 15 minutes over a window equal to a length of a fasting period, e.g., 8 hours in the example of FIGS. 7A-7C. For example, the median at 11 AM is the median of glucose data collected from 3 AM to 11 AM. The fasting period ends where the glucose median is a minimum. For example, in FIG. 7C the minimum glucose median is at 4.25 hours to 6.25 hours. The fasting period start time is equal to the fasting period end time minus the fasting period length (i.e., 8 hours). For example, as the fasting period end time is 4.25 hours to 6.25 hours, the fasting period start time is 8 hours earlier, and is between 20.25 and 22.25 hours. If the start and end times are in a range, any combination of start and end times within the range may be used to compute the fasting period. For example, the fasting period may be selected from a middle of the starting time range to the middle of the ending time range (21.25 hours to 5.25 hours). A bias term of Z hours may be added to the start and end times such that the fasting period starts at 21.25+Z hours and ends at 5.25+Z hours. The bias term may be based on a difference between the end of the fasting period as determined by autocorrelation and a time of a breakfast dose.

Rather than determining the length of the fasting period based on autocorrelation, the length of the fasting period may be determined to be a fixed period of time T, such as 8 hours. The glucose median may be computed based on T-hour windows, such that the time of day corresponding to the minimum glucose median indicates the end of the fasting period, and T-hours before the end time would be the start of the fasting period.

In another embodiment, one time series is defined for a fixed period and is used as a baseline day to perform a cross-correlation against the user's time series data. One time series may be glucose concentration over time, such as over 24 hours. However, it is understood that shorter or longer time periods may be used. The time resolution of the time period may depend on the time resolution requirement of the fasting period determination. For example, the time resolution may be hourly, or may be in fractions of hours, such as half-hour, quarter of an hour, or 1/10th of an hour, among other increments. Other time series may represent, for example, physical activity, activity monitoring readings, meal information, and insulin doses.

In an example, if the baseline day is based on a glucose time series spanning a fixed period at a time resolution, such as over a period of 24 hours in 20 minute increments, the user's glucose time series will be examined in the same 20 minute increment time resolution. While 20 minute increments are used as an example, it is understood that increments of other durations may be used, such as 10 minute, 15 minute, or 30 minute increments, among others. If the user's glucose time series is recorded at a different sample interval or contains gaps in data, an intermediate method can be used to interpolate the user's glucose time series into the same 20 minute increments. For example, by linear interpolation, quadratic interpolation, spline, or Savitzky-Golay filter. Glucose values in the baseline day represent a typical aspect of glucose variation over the fixed period with a specific range of time of day that is defined as the fasting period. Cross-correlation with the user's glucose time series generates an output from the cross-correlation that peaks at time points over the duration of the user's glucose time series indicating a most likely fasting period for every day of glucose time series evaluated.

In some embodiments, a change in schedule of the user may be determined based on reference to a user's connected schedule information. For example, the dose guidance system may have access to a user's calendar or planner. The calendar may indicate the user's time zone, and if the time zone has changed, or if the time has changed due to day light savings.

The change in schedule, and of the fasting period, may be determined by one or more of the methods described herein, and may be determined in combination with other methods for determining a fasting period. For example, the dose guidance system may determine a potential change in schedule based on a first method, and may use a second method to confirm the change in schedule. The estimated mealtimes or dose times may be determined based on the times determined by multiple methods described herein, and the dose times or mealtimes may be determined by an average, median, or weighted average, among others.

The detection of a schedule change can be used for dose classification when dose classification is based on the timing of a dose. For example, if the window for a dose to be classified as a breakfast dose is 6 AM to 10 AM, then for a 5-hour adjustment in the fasting period (used to determine a change in the user's schedule), the breakfast classification window may be adjusted to 11 AM to 3 PM. Thus, a dose taken in 11 AM to 3 PM, after detection of the schedule change, would be classified as a breakfast medication dose (rather than a lunchtime medication dose before the schedule change).

Some embodiments described herein relate to titrating medication doses based on glucose data segments that are specific to the user. As discussed above, titrating medication dosages based on predetermined TOD periods can present limitations when the patient's mealtimes (and medication dose times) deviate from the predetermined TOD periods. This problem is further compounded when the user's schedule changes such as due to a change in work schedule or when the user is traveling, or when the user otherwise has a varying meal schedule. Accordingly, systems and methods include segmenting glucose data associated with the patient's actual medication dose administration times, and analyzing the segmented glucose data to determine titrated medication doses. The segmented glucose data is subject to an event counting analysis to identify low glucose events or low glucose alarms. The event counting analysis may further include identification of frequent administration of correction doses following a particular mealtime dose. The segmented glucose data is subjected to a glucose pattern analysis as described herein. The titration determination is based on a combination of the event counting analysis and the glucose pattern analysis.

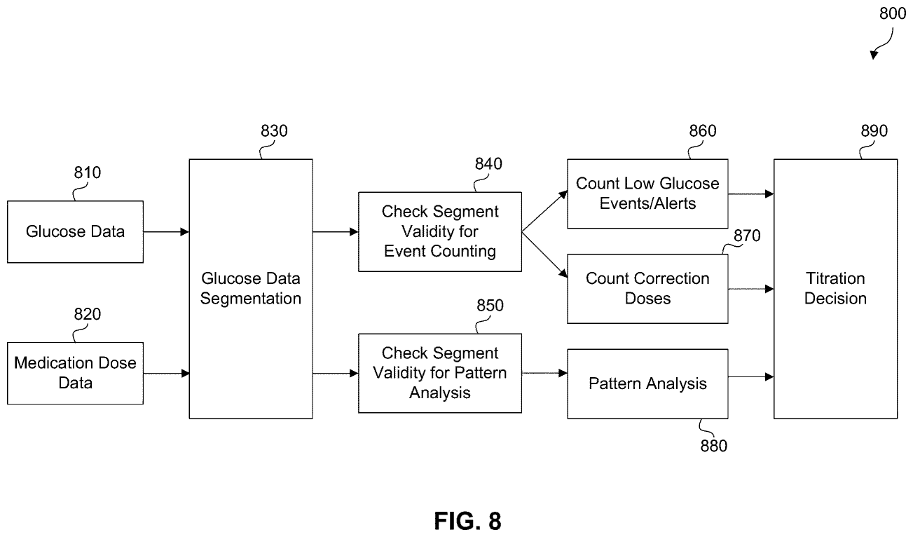

An exemplary embodiment is shown for example in FIG. 8. In glucose data segmentation 830, one or more processors receive glucose data 810 and medication data 820 for a user and produce dose-aligned segments of glucose data. The glucose data may be received from a glucose monitoring device, such as a continuous glucose monitor, in communication with the one or more processors. The medication data may be received from a medication delivery device, a dose monitoring device (e.g., a smart pen cap), or the medication data may be manually entered by the user. The medication data may include medication type, dose amount, and dose time, among other data. The glucose data segments are assessed for validity. There may be a separate validity assessment for the event counting analysis 840 and for the glucose pattern analysis 850. The glucose data segments for event counting are subject to the event counting analysis 860. The event counting analysis may count a number of low glucose events or low glucose alarms for each glucose data segment. The event counting analysis may alternatively or additionally include a correction dose counting analysis 870. The glucose data segments for glucose pattern analysis are subjected to the glucose pattern analysis to assess glycemic risks for each glucose data segment 880. A titration decision to increase, decrease or maintain the medication dose 890 is determined based on the results of the event counting analyses 860, 870 and the pattern analysis 880. The titration decision may be displayed to the user, such as by a display device.

In glucose data segmentation 830, the glucose data segments are classified based on their relationship to a medication dose. The classification may include classification of glucose data as either associated with a medication dose or glucose data not associated with a medication dose. The glucose data associated with a medication dose may be further divided into meal doses or correction doses. The meal dose may be further divided into breakfast doses, lunch doses, and dinner doses. The no dose classification may include an idle period associated with no dose. The idle period may further be divided into one or more overnight periods. The glucose data segment classification may be selected from the group of: breakfast, lunch, dinner, correction, idle, and overnight.

Dose-aligned glucose data segments are classified according to one or more classification rules. A glucose data segment for a first medication dose (meal dose or correction dose) is initiated at the time the first medication dose is administered and the glucose data segment is ended when a second or subsequent medication dose is administered. The glucose data segment may alternately be ended when a maximum time period is reached with no second medication dose administered. The maximum time period may be, for example, 5 hours.

A no dose segment, such as an idle segment, is started at the end of the maximum time period following a medication dose and the idle segment ends at the time a subsequent medication dose is administered. For example, if a first medication dose is administered at 7 AM, and the maximum time period is 5 hours, and no dose is recorded until 1 PM, the glucose data from 7 AM-12 PM will be classified as corresponding to a meal dose (e.g., a breakfast dose), and the glucose data from 12 PM to 1 PM will be classified as an idle period. The event counting analysis 860 may include glucose data segments classified as one of breakfast, lunch, dinner, correction, or idle.

The dose guidance system may determine if a dose is a correction dose or a meal dose using dose inference. As discussed, the meal doses may be further classified as breakfast, lunch or dinner doses. A user interface of the dose guidance system may receive a request from a user for dose guidance. The user may be prompted to select a type of dose, such as a breakfast dose, lunch dose, dinner dose or correction dose. If the dose is a breakfast dose, lunch dose, or dinner dose, the system may further determine either automatically (e.g., based on user input and glucose information) or manually (e.g., based on user input) if the dose has been administered as a late dose. The dose guidance system may recommend an amount of the dose based on parameters of the dose guidance system and user input information, such as a size or amount of a meal or timing of the meal. The dose guidance system may classify the dose according to the dose type selected by the user. However, relying on the user entry alone can be problematic as the user may request guidance but decide not to administer the dose, the user may modify the recommended dose amount, or the user may administer the medication dose at a later time.

If a medication dose is recorded as being administered by a connected medication injection pen of the dose guidance system, the system may use one or more criteria to confirm that the administered dose corresponds to the dose type selected by the user. The dose type selected by the user may be used to classify the dose if the amount of medication administered is equal to or close to (within a predetermined percentage) of the recommended dose. The dose type selected by the user may be used to classify the dose if the dose is administered within a predetermined amount of time from which the user selected the dose guidance. The dose type selected by the user may be used to classify the dose if the dose selection was made within a typical time for the meal. The dose guidance system may store a range of times for each meal. For example, the user may select a lunch dose, a dose is then administered, and if the dose is administered between a lunch time range of 11 AM to 3 PM, the system may record the dose is a lunch dose. If the dose type cannot be confirmed based on the above criteria, the user interface may prompt the user to confirm the dose type after the dose is administered. For example, a pop-up or notification may be displayed on the user interface requesting the user to confirm the dose type.

If the dose guidance system does not have a user interface, the dose classification may be inferred based on a time of administration of the dose. As discussed, the dose guidance system may store a typical time range for each meal, e.g., 11 AM to 3 PM for lunch, and infer that a dose administered during that time window for each meal corresponds to that meal. The time range may be based on TOD periods that are predetermined or that are determined by the dose guidance system as described herein.

In some embodiments, a machine learning model may be used to perform dose classification. Machine learning models can include, by way of example and not limitation, models trained using or encompassing decision tree analysis, gradient boosting, adaptive boosting, artificial neural networks or variants thereof, linear discriminant analysis, nearest neighbor analysis, support vector machines, supervised or unsupervised classification, and others. The machine learning model may be trained based on properly classified doses of the user, or of a population of users. The machine learning model may receive as inputs one or more of dose times, dose amounts, or glucose data, among other information. The model may predict if a dose is a correction dose or meal dose, and if the dose is determined to be a meal dose, the model may further predict whether the dose corresponds to breakfast, lunch or dinner.

For the pattern analysis 880, the same glucose data segments as determined for the event counting analysis 860 are used for the meal-time doses (e.g., breakfast, lunch, and dinner doses). The correction doses from the event counting analysis are removed from consideration for purposes of the pattern analysis 880. For the pattern analysis 880, overnight segments are determined from the idle segments of the event counting analysis 860. To determine an overnight segment, the system may analyze three consecutive glucose data segments according to the following rules. If the three consecutive glucose data segments include a series of: (any type of glucose data segment, Idle, Breakfast), then the Overnight data segment is determined to end at the end of the Idle period, and the Overnight data segment is determined to start a fixed number of hours before the end of the Idle period. The fixed number of hours may be 8 hours. Accordingly, if an Idle period ends at 7 AM when the breakfast dose is administered, the Overnight period is determined to be 11 PM-7 AM, where the fixed period is 8 hours.

If the three consecutive data segments include: (Dinner, Idle, Any type of data segment), then the Overnight data segment is determined to start at the Idle start time and the Overnight data segment is determined to end after a fixed amount of time following the Idle start time, such as 6 hours. For example, if the Idle start time is 12 AM, the Overnight period is determined to be 12 AM-6 AM.

If the three consecutive data segments include: (Lunch, Idle, Lunch), then the Overnight data segment is determined to end a fixed number of hours, e.g., 5 hours, before the idle period end time, and the Overnight data segment is determined to start fixed amount of time before the Overnight data segment end time, such as 8 hours. The Overnight data segment is determined as discussed above, and the system may further require the Overnight period to exceed a minimum amount of time, such as 6 hours.

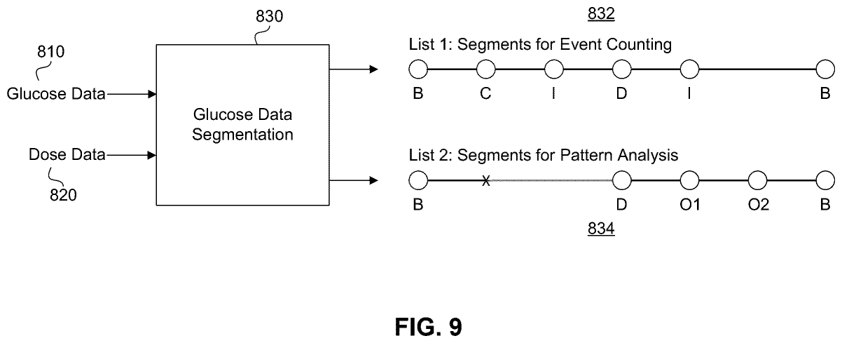

For example as shown in FIG. 9, the glucose data segmentation 830 resulted in glucose data segments for the event counting analysis 832 as Breakfast, Correction, Idle, Dinner, Idle and Breakfast the following day. The glucose data segmentation 830 resulted in glucose data segments for pattern analysis 834, based on the meal doses from the event counting analysis (Breakfast, Dinner and Breakfast), the Correction segment is removed, and the Idle segment is assessed for Overnight periods. Thus, the pattern analysis data segments 834 include Breakfast, Dinner, a first Overnight period, a second Overnight period, and Breakfast.

The glucose data segments are associated with a medication dose. The glucose data segments may be associated with a meal dose (such as breakfast, lunch, or dinner) or with a correction dose. A glucose data segment may be associated with a basal insulin dose if the basal insulin dose is administered within the prior 24 hours of the segment start time. Thus, a glucose data segment may be associated with a meal dose and a basal dose.

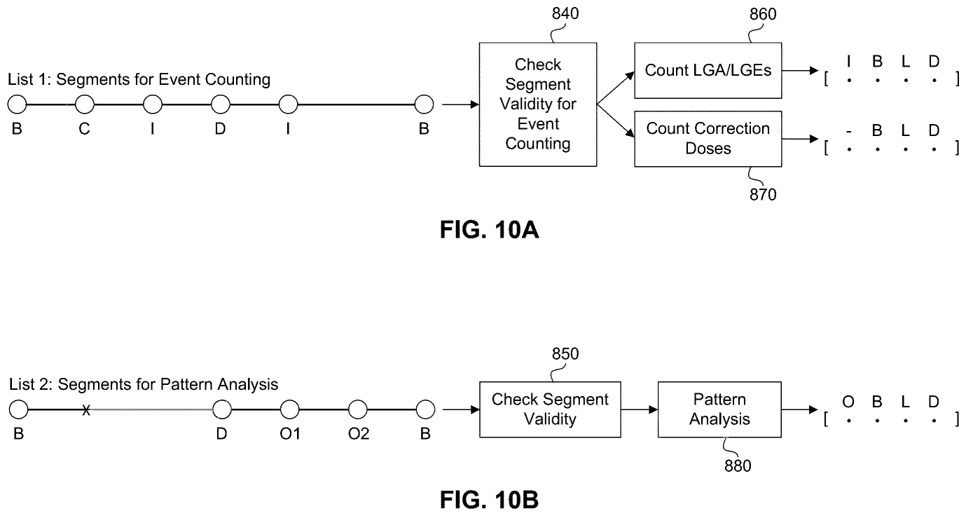

The segmented glucose data is subjected to the event counting analysis 860 (FIG. 10A). The event counting analysis 860 is configured to identify events that may have relevance to the titration determination. The events may include one or more of low glucose events (LGE), low glucose alarms (LGA), or post-meal correction doses. Additional events may be identified and used in the titration determination.

The glucose data segments are assessed for validity before proceeding to an event counting analyses 860, 870 or glucose pattern analysis 880. The validity assessment may differ for the event counting analyses 860, 870, and for the glucose pattern analysis 880. The validity assessment for the event counting analysis 840 may include determining if the glucose data segments are sufficiently recent. The validity assessment may include determining if a start time of the glucose data segment is within a predetermined amount of time of a most recent glucose data segment. Glucose data segments having a start time outside of the predetermined amount of time of the most recent glucose data segment are excluded from the event counting analysis. The predetermined amount of time may be, for example, 7 days, 10 days, or 14 days, among other amounts of time.

The dose guidance system may count the number of idle and meal segments in which a low glucose event occurred. The system determines for each glucose data segment if the low glucose event count is greater than a threshold number of low glucose events. The threshold number may be two low glucose events. The system may generate a low glucose event flag if the low glucose event count exceeds the threshold. The dose guidance system may alternatively or additionally count the number of idle and meal segments in which a low glucose alarm occurred. The system determines for each glucose data segment if the low glucose alarm count is greater than a threshold number of low glucose alarms. The threshold number may be four low glucose alarms. The system may generate a low glucose alarm flag if the low glucose event count exceeds the threshold. It is understood that the thresholds for determining a high number of low glucose alarms or events may be adjusted to other values.

The event counting analysis may count post-meal correction dose events 870. Frequent administration of post-meal correction doses may indicate insufficiency of the user's fixed medication doses. The system may count for each type of meal glucose data segment, the number of meal segments that are consecutively followed by a correction segment. If the count is greater than a predetermined threshold number, the system may determine a high correction count and generate a high correction count flag for the glucose data segment.

The glucose data segment validity is assessed for the pattern analysis 850. For the pattern analysis, the validity assessment may include determining a presence of gaps in the glucose data. Glucose data segments deemed to be invalid are not used in the pattern analysis, whereas glucose data segments deemed to be valid are used in the pattern analysis. The dose guidance system may deem a glucose data segment to be invalid if a number of missing glucose data values in the glucose data segment is greater than a threshold number of missing glucose data values. The glucose data segment may be deemed invalid of a number of consecutive missing glucose data values is greater than a threshold number of missing glucose data values. The threshold may be, for example, 2 consecutive glucose data values. The glucose data segment may be deemed to be invalid if there is no basal insulin dose recorded within 24 hours before the start of the glucose data segment. The glucose data segment may be invalid if there is no associated meal dose. This helps to ensure the glucose data segments are based on the user adhering to the medication therapy regimen. If there is no recorded medication dose for a glucose data segment, then the glucose data is not representative of the user's response to the medication dose, and as a result the glucose data is not properly considered for titrating the medication dose. The glucose data segment may be deemed to be invalid if the duration of the glucose data segment is less than a predetermined duration. The predetermined duration may be, for example, 3 hours.

The validity assessment for the pattern analysis 850 may further include a minimum amount of glucose data. The pattern analysis may be performed if there are at least 7 valid glucose data segments for each TOD period. This may help to limit uncertainty of the glucose data and ensure that the assessed patterns are accurate. The validity assessment for the pattern analysis may include a maximum amount of data. The pattern analysis may be based on a maximum number of glucose data segments for each TOD period. The maximum number of glucose data segments per TOD period may be, for example, 14 glucose data segments per TOD period. This helps to ensure that the pattern analysis, and titration determination is based on sufficiently recent data.

The pattern analysis is performed on List 2 (FIG. 10B). The detailed glucose pattern analysis is discussed herein, and as further disclosed in U.S. Publication No. 2021/0050085A1, incorporated herein by reference in its entirety. The pattern analysis includes calculating patterns and other relevant metrics for the TOD periods, such as breakfast, lunch, dinner and overnight periods. The pattern analysis further includes assessing sufficient data in each hour of each TOD period and adjusting to counteract metric estimation bias. Hour validity may be checked by using hours in a TOD period up to the median segment length for the analyzed TOD period.

The determination of the titrated medication dose 890 is based on a combination of the event counting analyses 860, 870 and the pattern analysis 880 (see, e.g., FIG. 8). The low glucose event or alarm counting analysis is used to determine whether to down-titrate the medication dose, i.e., to decrease the amount of the medication dose. If the number of idle periods with low glucose event flags is greater than an idle threshold (e.g., a threshold number of idle periods), the titration decision 890 is to decrease a basal insulin dose. If the number of idle periods with low glucose events is not greater than the idle threshold, but the number of breakfast doses, lunch doses, or dinner doses having low glucose events/alarms is greater than a meal threshold number (e.g., a threshold number of breakfast, lunch, or dinner periods), the titration determination is to decrease the associated meal dose amount. In some examples, the idle threshold is the same as the meal threshold. In other examples, the idle threshold is different than the meal threshold. In some examples, one or more of the breakfast, lunch, or dinner meal thresholds are the same. In other examples, each of the breakfast, lunch, or dinner meal thresholds are different.

The glucose pattern analysis 880 can be used to determine to up-titrate or down-titrate the medication dose, i.e., to increase or decrease the medication dose, as described in further detail in U.S. Publication No. 2021/0050085A1. A table of titration rules can be used to translate the glucose patterns and metrics into determinations to up-titrate, down-titrate or maintain the dose for each of the basal and meal doses. For each of the meal glucose data segments (e.g., breakfast, lunch or dinner segments), if the corresponding glucose pattern is High/Low, and there is a high correction count flag for the meal glucose data segment, dose guidance system may adjust the determined glucose pattern for that glucose data segment. For example, if the glucose data segment was determined to have the High/Low pattern, the High/Low pattern is replaced with a High pattern based on the high correction count flag. Otherwise, the assessed pattern for each glucose data segment is maintained, and the determination to up or down-titrate is based on the assessed pattern.

If the computed titration decision based on the low glucose event counting analysis 860 and the glucose pattern analysis 880 conflict, the titration decision may be based on the event counting analysis 860. That is, the event counting analysis takes priority over the glucose pattern analysis 880. This may help to prevent increasing medication dosage (based on the glucose pattern analysis) where a risk of hypoglycemia has been assessed based on the event counting analysis.

In some cases, a user may fail to enter event information or only partially enter event information, such as dose information or meal information, or the user may enter the event information incompletely. For example, a user may fail to record all doses administered during a day, or may fail to label a dose, e.g., as a breakfast dose. As a result, segmenting the glucose data to determine glucose data segments corresponding to a particular dose (or meal) is difficult when the event data is incomplete. If a minimum number of glucose data segments are not available, the system may be unable to make a titration decision, or the titration decision may be delayed until more data is received.

In some embodiments, the dose guidance system may use partial event data to determine glucose data segments. The system may integrate partial event data with a model of events occurring over time to allow for more robust data segmentation. The model represents the occurrence of events relevant to the glucose data segmentation over time. For example, when glucose data segments from TOD periods, e.g., post-meal or overnight periods, the model represents the sequence and timing of meals and/or insulin doses over time. The model may include expectations such as breakfast occurring in the morning, lunch occurring around noon, dinner occurring in the evening, and sleep occurring overnight. Additionally, the model may account for day-to-day repeat cycle of events.

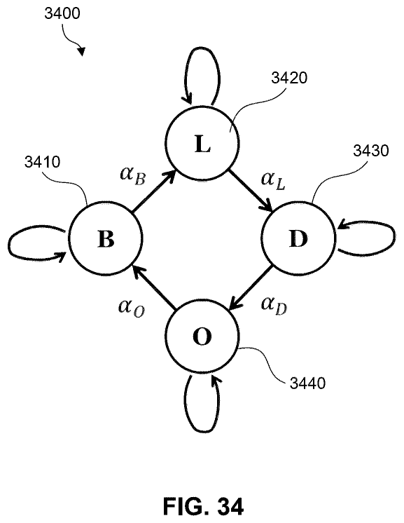

The model may include a sequence and timing of meals or doses. The model may be represented using a Markov Chain, as shown for example in FIG. 34. FIG. 34 shows a sequence or chain 3400 of four periods, a breakfast period (B) 3410, a lunch period (L) 3420, a dinner period (D) 3430, and an overnight period (O) 3440, with a indicating a probability of a transition to the next sequential period. The model tracks the period over time, and the model may transition sequentially between the plurality of periods. The plurality of periods may include mealtime periods and an overnight period. For example, the plurality of periods may include breakfast, lunch, dinner, and overnight periods as shown in FIG. 34. However, it is understood that additional periods (e.g., snacks or additional meals) or fewer periods (e.g., skipped meal(s)) may be included. The model determines that after a certain amount of time, the period transitions to the next sequential period. Each period may have a predetermined duration. The periods may have the same or different durations. For example, each of a breakfast period, lunch period, dinner period, and overnight period may have a duration of 6 hours. Alternatively, the periods may be different durations. For example, a breakfast period may be 5 hours, a lunch period may be 5 hours, a dinner period may be 5 hours, and an overnight period may be 8 or 9 hours. In another example, a breakfast period may be 4 hours, a lunch period may be 7 hours, a dinner period may be 5 hours, and an overnight period may be 8 hours. The duration of the periods may differ based on the user's actual or expected schedule. In a further example, if the user is fasting and is eating during a shortened window of time, a breakfast period may be 2 hours, a lunch period may be 3 hours, and a dinner period may be 3 hours, and an overnight or fasting period may be 16 hours. The model may include a probability of a transition between states at a given time.

To perform glucose data segmentation, the available event data is combined with the model's estimates. An initial state of the user is described by a vector of probabilities. The vector may include the probability that the user is in each of the plurality of periods. For example, the probability the user is in a breakfast, lunch, dinner, or overnight period may be initially set to 25% probability for each period as it is unknown which period the user is in. The user's state may be a function of time. The system may use any available event data to correct the user's state to a corrected state estimate. The user's state may be corrected forward in time. For instance, if a breakfast dose is observed, the user's state is breakfast immediately after the time of the breakfast dose, and is followed by the lunch period. The user's state can be corrected backward in time. For example, the user's state immediately before a recorded breakfast dose is the overnight state.

A dose guidance system may require a minimum number of glucose data segments in one or more TOD periods in order to titrate a medication dose corresponding for that TOD period (e.g., at least two glucose data segments in a post-breakfast period to titrate a breakfast dose). The use of partial event data to perform glucose data segmentation allows for identification of additional glucose data segments when complete event data is not available so that titration determinations can be made.

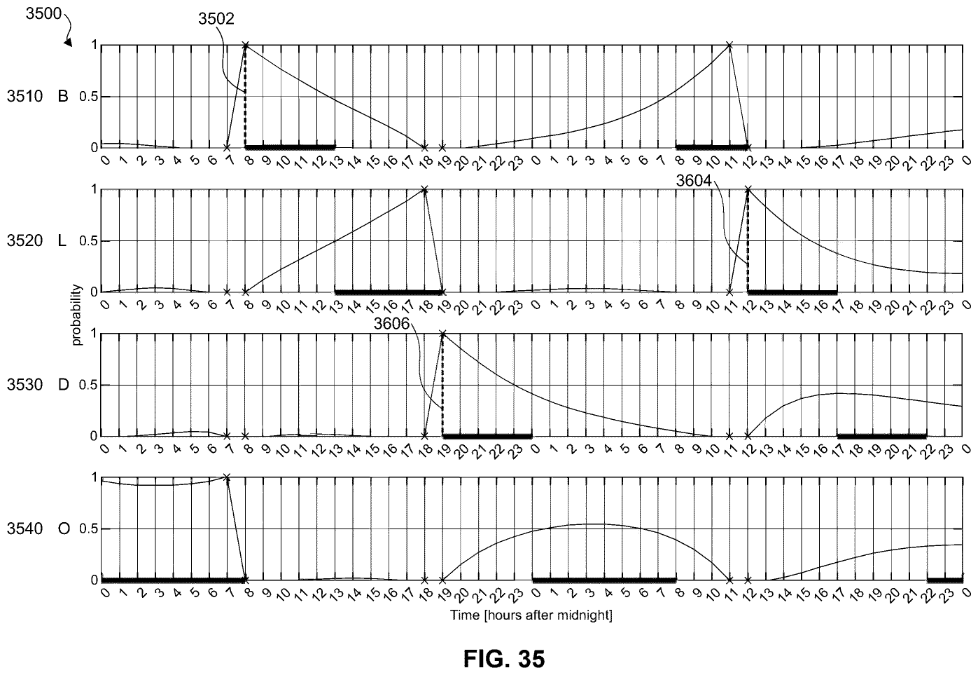

FIG. 35 shows plots 3500 showing a probability the user is in a given period of over time for each of a plurality of periods including a probability of a breakfast period over time 3510, a probability of a lunch period over time 3520, a probability of a dinner period over time 3530, and a probability of an overnight period over time 3540. In FIG. 35, a recorded dose is shown by a vertical dotted line, a TOD period is shown by a solid horizontal line, and a probability of a user's state corresponding to a given period is shown by a solid line or curve. For example, on a first day, a breakfast dose 3502 is recorded at 8 AM, and a dinner dose 3506 is recorded at 7 PM. On a second day, a lunch dose 3504 is recorded at 12 PM.

The system has incomplete event data, as the user has not recorded a meal dose for each meal each day. As a result, the system may not have sufficient glucose data segments to titrate the insulin doses. However, using the partial event data, the system can determine additional glucose data segments. The probability the user is in a given TOD period is very high at the time of an administered dose. The probability the user's state corresponds to that period decreases over time following the administered dose and the probability of the next successive period increases over time. Based on the partial event data in FIG. 35, the system may determine a high probability that a lunch period follows the breakfast period based on the recorded breakfast dose 3502, and that the lunch period ends at a time of the recorded dinner dose 3606. The system may determine a high probability that an overnight period follows the recorded dinner period 3606 on the first day. The system may determine that an overnight period ends at the time of the recorded breakfast dose 3502 on day one, e.g., 8 AM. The system may determine a high probability that a breakfast period on day two starts at the same time as the breakfast period on day one, and terminates at the time of the recorded lunch dose 3604. In this way, the recorded event data, e.g., a meal or medication dose, can be used to improve the estimate of the user's state and the identification of the TOD period.

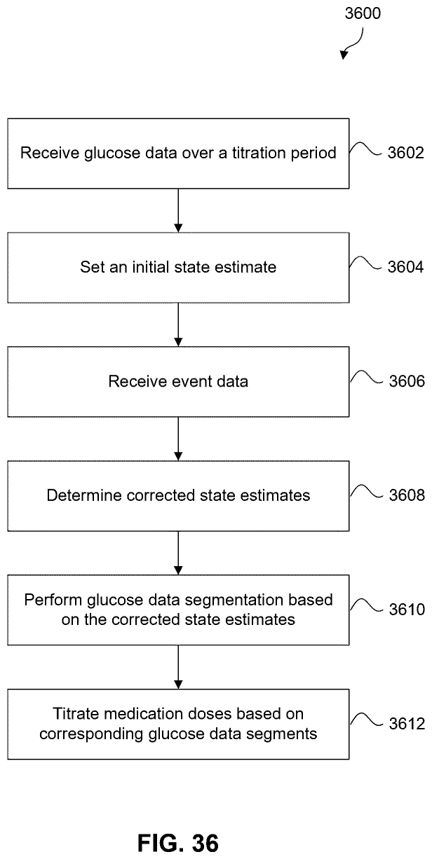

An exemplary method of titrating a medication dose and performing glucose data segmentation based on partial event information is shown in FIG. 36. The method 3600 may include receiving glucose data over a titration period 3602. The method includes storing or setting an initial state estimate corresponding to a probability that the user is in one of a plurality of TOD periods 3604. The current state is assumed to progress sequentially from a first TOD period to a subsequent TOD period over time. Event data is received 3606 and is used to determine corrected state estimates 3608. For example, the event data may include a recorded meal or a recorded insulin dose. The user may tag the meal or insulin dose as corresponding to a particular meal, e.g., breakfast, lunch or dinner. The recorded meal or insulin dose may be time-stamped so that the time of the meal or insulin dose is known. Glucose data segmentation may be performed based on the corrected state estimates 3610. The glucose data for each of the periods may be used to titrate the corresponding dose 3612 as discussed herein. For example, glucose data segments corresponding to the breakfast period may be used in a determination to titrate the breakfast dose.

Some embodiments described herein relate to improved methods for titrating medication (e.g., insulin) doses. Titration of doses, such as whether to increase or decrease the next dose of a medication, may rely on estimates of hyperglycemia or hypoglycemia. For example, where the risk of hypoglycemia is high, as may be evidenced by a pattern of low glucose levels following a first dose of medication, a dose guidance algorithm may recommend a lower dose as the second dose to reduce or avoid hypoglycemia. If the risk of hypoglycemia is moderate or low, the dose guidance algorithm may not recommend a dose change and may maintain the medication dose at the current level. If the risk of hyperglycemia is high, as evidenced by a pattern of high glucose levels, the dose guidance algorithm may recommend increasing the dose of medication administered by the patient to help reduce or avoid hyperglycemia.

When an increase in dose is recommended, titration may increase the amount of the dose at a fixed increment, such as an increase of a fixed percentage or an increase of a fixed amount of the medication (e.g., increase insulin by 1 U). As titration of the dose may occur on a weekly basis or even less frequently, by adjusting the dose amount on a fixed basis the titration process may proceed very slowly. This can be undesirable as the patient may continue to experience poor glucose control while the dose guidance algorithm is gradually adjusting the dose amount, and may result in delay of therapy escalation if it is determined that a second medication is needed once the first medication is fully or optimally titrated. Further, as the glucose levels approach target glucose levels, titrating the dose in a fixed amount may overshoot the optimal medication dose. Thus, as the optimal dose is approached, it would be desirable to make relatively small adjustments to the dose amount rather than continue to make the same fixed dose adjustment.

Rather than recommending a fixed dose adjustments, systems and methods described herein may recommend an amount of the dose adjustment to allow titration to proceed more rapidly. If the user's glucose levels are very high, the user may be able to accommodate a larger increase in the medication dose to lower glucose levels into a target range and to avoid hyperglycemia. Further, as the user approaches euglycemia, the amount of the dose increase may become relatively small. Thus, the dose adjustment may be proportional to the proximity of the user's glucose levels to a target level or range, or based on the risk of hypoglycemia. The dose adjustment may be proportional to the hypoglycemia risk, and a greater adjustment to the dose may be made when the risk of hypoglycemia is low.

The amount of the dose increase can be determined based on a measure of central tendency of the glucose levels, such as glucose median. While the disclosure may refer primarily to “glucose median” it is understood that other measures of central tendency of glucose values may be used, such as an average glucose level. The amount of the dose increase can be based on comparison of the glucose median to a hypoglycemia risk threshold. The hypoglycemia risk threshold may be a hypoglycemia risk curve on a plot of glucose central tendency (e.g., glucose median) over glucose variability. The glucose variability may be defined as a difference between a tenth percentile of glucose levels and the glucose median. However, other measures of variability may be used such as coefficient of variation, standard deviation, and interquartile range, among others.

The hypoglycemia risk curve may be defined as described in U.S. Publication No. 2020/0015756, which is incorporated herein by reference in its entirety. The hypoglycemia risk curve may be a set of points on a plot of glucose central tendency and variability having the same value for a hypoglycemia risk metric. The hypoglycemia risk metric may be AU70. AU70 is dependent on both time and magnitude of glucose readings below the 70 mg/dL—this metric is referred to as AU70 (short for “area under 70 mg/dL”). The AU70 metric is defined as: a) Sum of all differences (70 mg/dL—Reading) for all Readings below 70 mg/dL, b) Divided by number of all Readings. However, in alternate embodiments, different hypoglycemia risk measures may be used.

A particular population of glucose data can be modeled by a Gamma distribution. This distribution is uniquely defined by the glucose median and glucose variability determined from the data population. The glucose median and glucose variability metrics define a point on the median-variability plot. Each point on this plot has an associated value for the AU70 metric determined by:

$$A = {\int }_{0}^{70} ( 70 - G ) Gamma ( G ; a , b ) dG \qquad ( 1 )$$

Here, Gamma (G; a,b) is Gamma distribution, G is the glucose value in mg/dL, and a and b are the Gamma distribution shape and scale parameters. The parameters a and b uniquely specify the Gamma distribution. For the present method, specifying the median and the glucose variability also similarly specifies the Gamma distribution uniquely. A curve made up of points associated with constant AU70 value can be determined analytically using the above equation. This curve is called the “Hypo Risk Curve” and each curve is associated with a particular AU70 value.

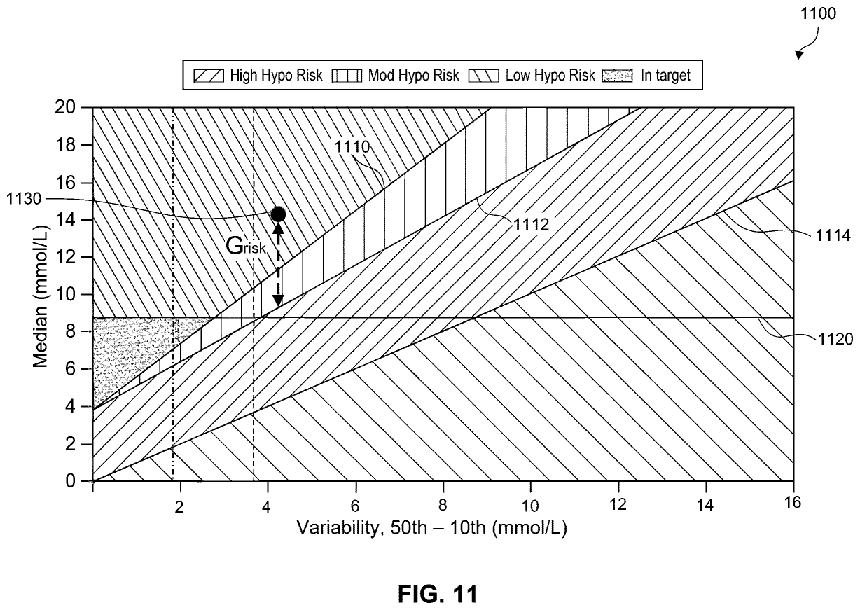

A hypoglycemia risk metric, Grisk, may be defined as a vertical distance from the glucose median to a hypoglycemia risk curve on the plot of glucose median over glucose variability as shown for example in FIG. 11. One or more hypoglycemia risk curves may divide the glucose median and variability plot into zones of relative hypoglycemia risk. In FIG. 11, three hypoglycemia risk curves are shown. Specifically, a low hypo risk curve 1110, a moderate hypoglycemia risk curve 1112, and a high hypoglycemia risk curve 1114. A point 1130 corresponding to the user's glucose variability and glucose median for a TOD segment can be plotted. A vertical distance from the point 1130 to the moderate hypoglycemia risk curve 1112 can be drawn, and a length of the line or the distance between the point 1130 and the hypoglycemia risk curve is the Grisk value. Grisk may be expressed in mg/dl or mmol/ml. The greater the Grisk value, the greater the dose amount may be increased.

The specific dose adjustment may be based on comparison of Grisk to a series of bins or thresholds. The amount of dose increase may be an absolute value or a percentage of the most recent dose. For example, if Grisk is less than a first threshold, the dose is increased by a first percentage. If Grisk is greater than the first threshold and less than a second threshold, the dose is increased by a second percentage that is greater than the first percentage. If Grisk is greater than the second threshold, the dose is increased by a third percentage that is greater than both the first and second percentages. It is understood that additional or fewer bins may be created, and that the percentage increases may be varied from the disclosed example. Table 1 presents an example of Grisk bins and the associated dose adjustment.

TABLE 1
Exemplary Dose Increases for Gᵣᵢₛₖ bins.

| Gᵣᵢₛₖ bin | Percentage increase in dose |
|---|---|
| Gᵣᵢₛₖ < 5 mg/dl | 5% |
| 5 mg/dl < Gᵣᵢₛₖ < 15 mg/dl | 10% |
| Gᵣᵢₛₖ > 15 mg/dl | 15% |

The dose increase may be linearly related to Grisk. For example, Gᵣᵢₛₖ may be grouped into three bins based on the Gᵣᵢₛₖ value in relation to a Gᵣᵢₛₖ lower threshold Gr_{lower} and a Gᵣᵢₛₖ upper threshold Gᵣᵤₚₚₑᵣ. Each bin may have a corresponding dose adjustment as follows, where prIn_{lower} is a lower percent increase, and prInᵤₚₚₑᵣ is a higher percent increase:
if Gᵣᵢₛₖ<Gr_{lower}, then increase dose by prIn_{lower};
if Gr_{lower}<Gᵣᵢₛₖ<Gᵣᵤₚₚₑᵣ, then increase dose by prIn_{lower} to prInᵤₚₚₑᵣ, based on the formula:

$$\% increase in dose = {prIn}_{lower} + \frac{( {G}_{risk} - {G}_{rlower} ) * ( {prIn}_{upper} - {prIn}_{lower} )}{( {G}_{rupper} - {G}_{rlower} )}$$

if Gᵣᵢₛₖ>Gᵣᵤₚₚₑᵣ, then increase the dose by prInᵤₚₚₑᵣ.

For example, Gr_{lower} is 5 mg/dl, Gᵣᵤₚₚₑᵣ is 15 mg/dl, prIn_{lower} is 5%, and prInᵤₚₚₑᵣ is 20%.

A Gᵣᵢₛₖ may be determined for each TOD period. For example, the TOD periods may include periods corresponding to breakfast, lunch, dinner, and overnight periods. Each TOD period may have the same Gᵣᵢₛₖ thresholds or bins, or may have different thresholds or bins, and the bins for different TOD periods may have the same or different dose adjustments.

Some patients may administer a medication regimen including a basal insulin dose and one or more meal doses. For example, the medication regimen may include a basal dose, a breakfast dose, a lunch dose, and a dinner dose. Titration of the patient's medication regimen may be prioritized in the order of basal dose, breakfast dose, lunch dose, and dinner dose, with increases to basal doses determined before any meal dose increases, breakfast dose adjustments determined before lunch or dinner dose adjustments, and lunch dose adjustments determined before dinner dose adjustments. For example, if the user's glucose levels are high, or are assessed to have a High pattern in a glucose pattern analysis in each of the TOD periods, only the basal dose is increased. If the breakfast dose shows a High pattern, the breakfast dose is increased irrespective of the lunch or dinner period glucose patterns (as long as the overnight glucose pattern is not high). The lunch dose is increased when only the lunch or lunch and dinner TOD periods show a High glucose pattern.

Increasing a single medication dose at a time may delay achieving full or optimal titration and delays glucose control. This may be particularly problematic when the user is severely underinsulinized and requires increased insulin doses for multiple medication doses. Accordingly, it would be desirable to titrate by simultaneously increasing multiple medication doses. However, the risk of increasing multiple doses simultaneously is overdelivering insulin which may result in increased instances of hypoglycemia. Thus, there is also a need to avoid overdelivering insulin and increasing hypoglycemia when simultaneously titrating multiple medication doses.

Increasing the basal dose has a glucose lowering effect on all time periods of the day. As a result, simultaneously increasing the basal dose and a meal dose may result in increased risk of hypoglycemia in the time period of the day corresponding to the meal dose. In addition, increasing a meal dose may have a glucose lowering effect on the subsequent time periods of the day. For example, increasing the breakfast dose may have a glucose lowering effect on the lunch time period of the day because increasing the breakfast dose may lower the starting glucose for the lunch time period.

In order to allow for simultaneous increases in multiple doses while mitigating hypoglycemia risk, the determination to simultaneously increase multiple doses may be based on hypoglycemia risk, such as based on Gᵣᵢₛₖ. The determination to increase multiple doses may be based on comparison of Gᵣᵢₛₖ values from different TOD periods having High patterns. Increasing a meal dose requires the corresponding Gᵣᵢₛₖ value to be greater than the Gᵣᵢₛₖ value of prior time periods with dose increases. For example, in order to increase a basal dose and a breakfast dose simultaneously (when the overnight and breakfast glucose patterns are High patterns), breakfast Gᵣᵢₛₖ may be required to be greater than the overnight Gᵣᵢₛₖ. To increase all three meal doses simultaneously (when the breakfast, lunch, and dinner TOD periods have High patterns), the dinner TOD period may be required to have the highest Gᵣᵢₛₖ of the three meals, followed by the lunch TOD period, and followed by breakfast TOD period.

To reduce the risk of hypoglycemia from simultaneous dose increases as described above, the meal dose increase may be modified by a factor based on the relative Gᵣᵢₛₖ values. The meal dose increase is lowered if the corresponding Gᵣᵢₛₖ is less than Gᵣᵢₛₖ in the prior TOD periods with dose increases. For example, if dose amounts are based on binning Gᵣᵢₛₖ for different TOD periods as described herein, and the basal dose is increased by 10% and the breakfast dose is increased 10% based on the binning, to simultaneously increase both doses, the breakfast dose may be increased by only 5% instead of 10% if the breakfast Gᵣᵢₛₖ is less than the overnight Gᵣᵢₛₖ. In other embodiments, the meal dose increase may be lowered irrespective of Gᵣᵢₛₖ values when simultaneously titrating with a basal dose or prior TOD period meal doses.

A dose guidance system may decrease a basal insulin dose and/or may decrease one or more meal doses when the risk of hypoglycemia is determined to be high. However, the estimated risk of hypoglycemia may not be accurate if the user consumes “rescue” carbohydrates in response to low glucose alarms, such as may be provided by a continuous glucose monitoring system. This can result in overtitration of the insulin dose which may cause an increased number of alarms, increased rescue carbohydrate intake, and unnecessary administration of additional insulin doses.

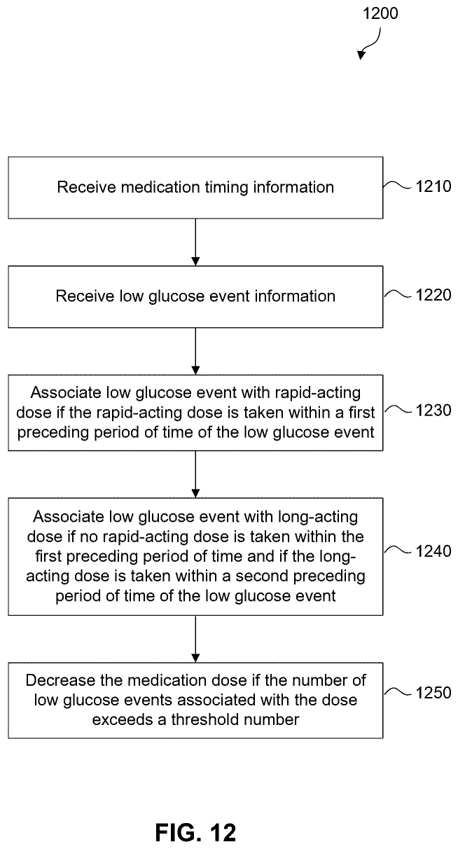

Some embodiments described herein relate to systems and methods for titrating a medication dose based on a number of low glucose events as shown in FIG. 12. The system may receive medication information 1210. The medication information may include, for example, the type of medication, time of administration, and amount or dose of medication administered. The system may further receive low glucose event information 1220. The low glucose event is determined to be associated with a meal dose, also referred to as rapid-acting dose, if the rapid-acting dose is taken within a first preceding period of time of the low glucose event 1230. The low glucose event is determined to be associated with a basal dose, also referred to as a long-acting dose, if there is no rapid-acting dose within the first preceding period of time and the long-acting dose is taken within a second preceding period of time of the low glucose event 1240. The respective medication dose is decreased if the number of low glucose events associated with the medication dose exceeds a threshold number of low glucose events 1250.

The dose guidance system may receive medication information from a medication delivery device, such as an infusion pump or a smart injection pen. The medication information may be received from a dose monitoring device, such as a smart pen cap attached to an injection pen. The medication information may be received manually from the user at an input of a display device of the dose guidance system.

The dose guidance system may receive the glucose alarm data or the glucose level data from a glucose monitoring device, such as a continuous glucose monitor. The dose guidance system may count the number of glucose alarms or may analyze the glucose data to identify low glucose events as discussed above. The low glucose events may be based on a number of low glucose alarms output by a continuous glucose monitor. The low glucose events may be based on the glucose data, where a low glucose event may be defined by a predetermined minimum number of glucose readings below a predetermined low glucose level within a predetermined period of time. For example, the low glucose event criteria may be two or more glucose levels below 70 mg/dl within a 15 minute time period. However, it is understood that the number of glucose levels, the glucose level threshold, and the time period used to define a low glucose event may be adjusted from these values.

A low glucose event is associated with a medication dose if it occurs within a first period of time following administration of the medication dose. When the medication dose is a meal dose, such as a rapid-acting insulin dose taken at breakfast, lunch or dinner, the first predetermined period may be in a range of 2 to 8 hours following the medication dose, 3 to 7 hours, or 4 to 6 hours. The predetermined period may be 5 hours (or any other period of time). A low glucose event is associated with a basal dose if it is not within the first predetermined period of the meal dose, and is within a second predetermined period following the basal dose. The second predetermined period may be, for example, 24 hours.

The medication dose is decreased if the number of low glucose events associated with the medication dose exceeds a predetermined number of low glucose events. For example, the predetermined number of low glucose events may be 4 low glucose events in a 7 day period. Each medication dose may have a different threshold. For example, the threshold for the basal dose may be a first number while the threshold for the meal doses may be a second number. In a further example, the predetermined number of low glucose events may be 4 low glucose events for a breakfast dose, and may be 5 low glucose events for a lunch dose.

In some embodiments, if titration of the medication dose is based on other inputs, the dose decrease recommendation resulting from counting of low glucose events may override the dose change recommendation based on other inputs, such as a dose change recommendation based on a glucose pattern analysis for each TOD period.

In some embodiments, the dose guidance system may require sufficient glucose and medication data to be present for all TOD periods in order to determine a dose change recommendation. The dose guidance algorithm may require an identified glucose pattern in each of the TOD periods in order to determine a dose change recommendation. This helps to confirm the accuracy of the identification of patterns in the user's glucose data and medication regimen. For example, the dose guidance algorithm may require 7 days of medication data and glucose data for each TOD period (e.g., for each of breakfast, lunch, dinner and overnight periods) in order to recommend an increase or a decrease to any medication doses, e.g., the basal dose or any meal-time doses.

However, sufficient glucose data or identified glucose patterns for each TOD period may not be available if a patient misses a medication dose (e.g., the patient forgot to take a lunch dose), or when there is a gap in the glucose data (e.g., the patient's glucose monitor fell off, expired without replacement, or was out of range of the associated display device). Patients may habitually miss certain doses, such as habitually missing a breakfast dose. As a result, titration of any medication doses may be delayed by several days or more until a sufficient amount of data is collected for all TOD periods. This may result in delay of full titration and a longer time in which the patient experiences poor glucose control. This can be particularly dangerous where the available data indicates a pattern of hypoglycemia in one or more of the TOD periods that requires adjustment of the dose for that TOD period. Thus, there is a need to safely titrate medication doses more rapidly.

To avoid delaying adjustment to medication doses, the dose guidance system may have relaxed requirements for titrating a medication dose when decreasing a medication dose, but may still require sufficient data for all TOD periods when increasing the medication dose. For example, the dose guidance system may require a first number of days of medication data and glucose data to recommend a decrease to a medication dose, but may require a second number of days of medication data and glucose data to recommend an increase in the medication dose. The second number of days is greater than the first number of days. The first number of days may be in a range of 4 to 8 days (or any other predetermined number of days), and may be for example 5 days. The second number of days may be in a range of 6 to 10 days (or any other predetermined number of days), and may for example 7 days. The first or second number of days may refer to a consecutive number of days, such as 4 days in a row. The first or second number of days may refer to a number of days in a given time window, such as 5 days in a 7 day period. In another example, the dose guidance system may require a first number of days of data in a TOD period to recommend a decrease in the associated medication dose irrespective of the amount of data collected in other TOD periods. For example, if there are 7 days of data in the breakfast period that indicate a decrease in the breakfast dose is needed, the dose guidance algorithm may recommend a decrease in the breakfast dose despite the lunch period not yet having 7 days of data.

The dose guidance system may require all TOD periods to have sufficient data to recommend an increase in a basal dose, but may relax requirements for the amount of data to increase a meal dose. For example, an increase in a dinner dose may require a sufficient number of days of glucose and medication data be available for dinner and overnight periods, irrespective of the availability of data for the breakfast and lunch periods. An increase in a meal dose for one TOD period may require sufficient data for that TOD period and for the next consecutive TOD period, irrespective of the amount of data available for the remaining TOD periods.

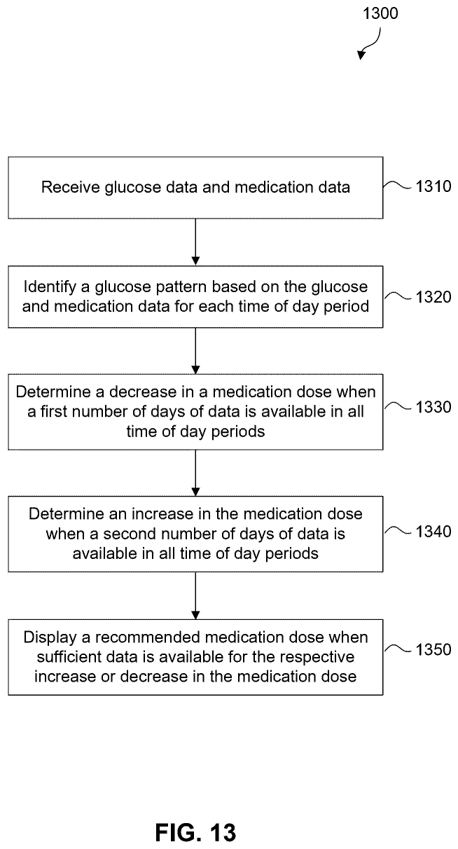

An exemplary rapid titration method 1300 is shown in FIG. 13. The dose guidance system may receive glucose data and medication data over a period of time 1310. The glucose data may be received by a glucose monitoring device, such as a continuous glucose monitor worn by a user and having a first portion positioned under the skin and in contact with a bodily fluid, and a second portion arranged above the skin and coupled to sensor electronics as described herein. The dose guidance system may receive the glucose data at a display device in communication with the continuous glucose monitor, at a remote computer, server or cloud, or both. The dose guidance system may identify a glucose pattern based on the collected glucose data and medication data for each TOD period 1320. TOD periods may correspond to mealtimes. TOD periods may include breakfast, lunch, dinner, and overnight periods. Additional or fewer TOD periods may be included as discussed herein. If the system determines to decrease a medication dose associated with a TOD period based on the identified glucose pattern for the TOD period, the system may require a first number of days of data to be available 1330. If the system determines to increase a dose associated with a TOD period based on the identified glucose patterns, the system may require a second, greater number of days of glucose data and medication data to be available 1340. The system may display the recommendation to increase or decrease the medication dose when a sufficient amount of data is available 1350.

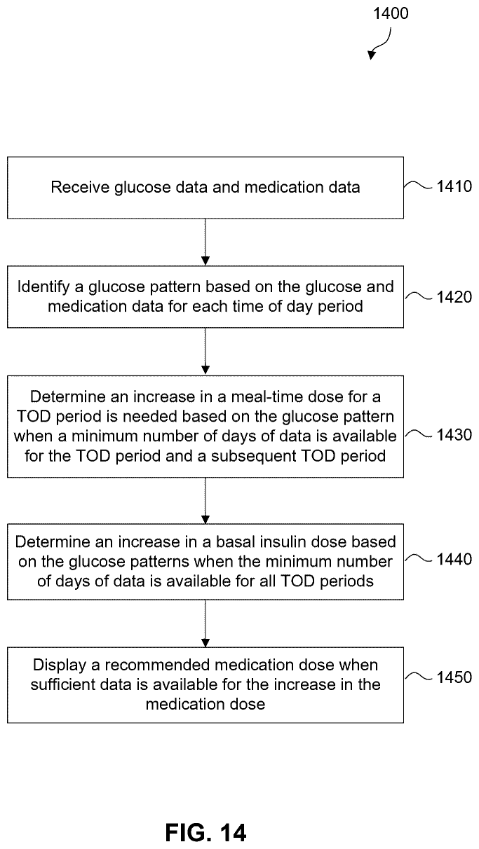

An exemplary method of determining a dose adjustment 1400 is shown in FIG. 14. The dose guidance system may receive glucose data and medication data 1410. The system may identify a glucose pattern based on the collected glucose data and medication data for each TOD period 1420. If the dose guidance system determines an increase in a meal-time dose is needed based on the identified glucose patterns for the respective TOD period, the system may require a minimum number of days of data to be available for the respective TOD period and a subsequent TOD period 1430. If the dose guidance system determines a basal dose needs to be increased based on the identified glucose patterns, the system may require the minimum number of days of data to be available for all TOD periods 1440. The minimum number of days may be in a range of 5 to 10 days, and may be 7 days. The system may display the recommendation to increase the medication dose when a sufficient amount of data is available 1450.

Dose guidance systems may be used by a patient to help manage the patient's glucose levels. The dose guidance systems may require initial set up and configuration by a health care practitioner (HCP). However, HCPs may have little time to help users to perform the initial set up and to configure the dose guidance systems. Further, many HCPs may lack the specialized knowledge or training needed to configure the dose guidance system for optimal benefit of the patient. Accordingly, there is a need to simplify the initial set up and configuration process of the dose guidance system to reduce the amount of time and the level of knowledge needed by the HCP.

A dose guidance system may include a set of equations used to calculate a dose, such as a bolus calculator. The bolus calculator may have one or more parameters used in determining a dose recommendation, including but not limited to an insulin sensitivity, correction factor, carbohydrate ratio, duration of insulin action, and maximum bolus, among other parameters. The user may have to manually enter values for each of the parameters during the initial set up and configuration of the dose guidance system.

A dose guidance system may alternately or additionally include a set of fixed doses to be administered by the user. For example, the set of fixed doses may include a basal dose and one or more meal doses, such as a breakfast dose, a lunch dose, and a dinner dose. The basal dose may be a single dose or may include multiple doses, e.g., two doses. The user may have to select or enter values for each dose of medication to be administered during the initial set up and configuration of the dose guidance system.

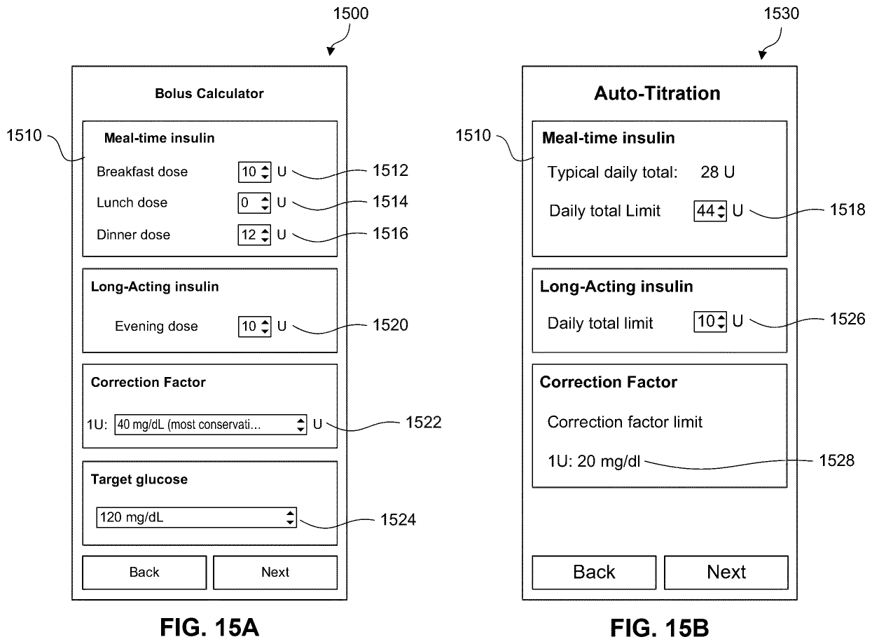

An exemplary user interface for a dose guidance application for a user mobile device is shown in FIGS. 15A and 15B. User interface 1500 may be displayed for example on a mobile device, such as a smartphone, of the user. As shown in FIG. 15A, the dose guidance system may display a user interface 1500. The user interface 1500 may be configured to receive user entry of medication doses. User interface 1500 includes a meal-time insulin section 1510 for setting the initial values for insulin doses for each meal. For example, a breakfast dose setting 1512, a lunch dose setting 1514, and a dinner dose setting 1516, wherein the setting may be zero if the user does not take a medication dose for that meal. The user interface 1500 includes a setting for a long-acting or basal insulin dose 1520. The user interface 1500 includes a setting for a correction factor 1522. The user interface includes a setting for a target glucose level 1524. The settings are shown as being selectable from a list, however, in other embodiments, the user may have to manually enter a numerical value for each setting. Initial values may be populated by the system to default settings that can be adjusted by the user.

In FIG. 15B, a user interface 1530 is shown for the auto-titration settings. The dose guidance system may adjust the initial parameters for the medication dose amounts, correction factor, and target glucose based on analysis of the user's glucose data over time. User interface 1530 allows the user to set limits to the dose guidance algorithm, such that the titration proceeds within the bounds set by the limits. The meal-time insulin or rapid-acting insulin may include a limit on the total daily dose of rapid-acting insulin 1518. The total daily dose is the sum of all rapid-acting insulin doses taken in one day. One day may be from midnight of a first day to midnight of a second day, or may be another 24-hour period. The dose guidance algorithm may freely titrate the doses for each meal so long as the total daily dose does not exceed the set limit 1518. Similarly, the long-acting insulin may have a total daily dose limit 1526. The dose guidance algorithm may freely titrate the long-acting insulin dose (or doses) such that the total long-acting dose does not exceed the set limit. The correction factor may have a limit 1528 that is a minimum correction factor. As the correction factor represents the change in glucose level per unit of insulin, a lower correction factor indicates that more insulin is needed to achieve a given change in glucose level, and thus a low correction factor results in delivery of more insulin than a high correction factor.

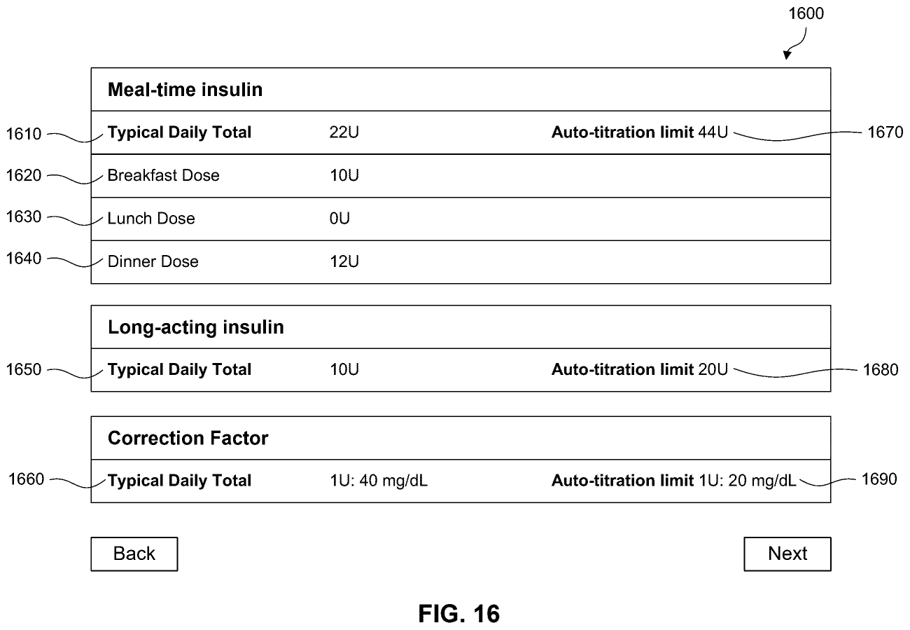

Another exemplary user interface for configuring a dose guidance system is shown in FIG. 16. The dose guidance system may present a user interface 1600 on a web application, such as may be displayed on a computer or laptop of a HCP. The HCP may configure the dose guidance system and the configuration entered by the HCP on user interface 1600 may be communicated to a dose guidance application executed on a display device of the user. The settings configured by the HCP on web application 1600 may be communicated directly to the dose guidance application of the user, or may be communicated to a remote computer, server, or cloud, and in turn the dose guidance application receives the configuration entered by the HCP from the remote computer, server or cloud.

The user interface 1600 may include one interface with all parameters to be set to configure the dose guidance system. The user interface 1600 may separate the settings into mealtime or rapid-acting insulin doses and long-acting or basal insulin doses. The user interface may include titration limits for one or more of the medication doses. A meal-time insulin section may include a setting for a total daily dose 1610 for all meals. The total daily dose (TDD) 1610 may be entered by the HCP, or may sum the entered values for the meal doses. The meal-time insulin section may include individual entries for each meal-time dose, such as for a breakfast dose 1620, lunch dose 1630, and dinner dose 1640. The user interface may include a setting for a TDD of long-acting or basal insulin 1650. The user interface may include a setting for the correction factor 1660.

The system may use the user-entered values as initial values, and the initial values may be titrated by the system over time to improve glucose control. The user interface 1600 may prompt the user to enter limits for one or more of the medication doses. In FIG. 16, a limit includes a maximum TDD for all meal-time doses 1670 (“auto-titration limit”), such that the system will titrate the TDD and individual meal-time dose amounts up to the maximum TDD for all meals, and will not recommend dose amounts that would exceed the TDD for all meal-time doses. A limit may alternatively or additionally be set for titration of the long-acting insulin dose 1680. The limit may be a maximum long-acting insulin dose. The system will titrate the long-acting dose up to the maximum long-acting insulin dose, and will not recommend a long-acting insulin dose above the maximum long-acting insulin dose setting. A limit may alternatively or additionally be set for a minimum correction factor 1690. The system may titrate the correction factor, but will not recommend a correction factor below the minimum correction factor.

The limit for the correction factor or long-acting insulin dose may be determined based on the limit set by the user for the TDD of rapid-acting insulin. For example, the TDD of rapid-acting insulin may be set as a percentage increase relative to the current or initial TDD of rapid-acting insulin. The limit on the long-acting insulin dose or the correction factor may be automatically set to the same user-selected percentage. For example, if the user selects a limit for the TDD of rapid-acting insulin doses to 50% more than the current sum of the fixed rapid-acting doses, the dose guidance system may automatically set a limit of the long-acting insulin dose to the same percentage, i.e., 50% more than the current long-acting dose, and/or the correction factor(s) to the same percentage, i.e., 50% less than the current correction factor.

A user-entered value for a first parameter may be used by the dose guidance system to automatically select a value for a second parameter. In this way, the system may save the user the time and effort of setting each parameter individually. Dose guidance system may prompt a user to enter a target glucose level. The target glucose level may be used, for example, in a bolus calculator to calculate a dose of insulin to administer. The target glucose level may be selected from a list of one or more options, such as 100, 110, 120, 130, or 140 mg/dl, among others. Lower target glucose levels are considered to be more aggressive in that the system delivers more insulin to achieve the lower target glucose than for a relatively high target glucose level.

A median glucose goal may be used by a dose guidance algorithm of the dose guidance system to determine optimal fixed medication doses. A lower value for glucose median goal would result in titration toward larger insulin doses in order to achieve the low glucose median goal. Selection of a target glucose level for bolus calculations may result in automatic selection of a glucose median goal for titration. Target glucose level may be more readily understood by HCPs than glucose median goal. Further, tying selection of glucose median goal to target glucose level helps to simplify the set-up of the dose guidance system. The dose guidance system may store a corresponding median goal setting for each target glucose level. The settings may be stored in a table in memory of the dose guidance system. For example, a selection of 110 mg/dl results in a median goal setting of 140 mg/dl, a target glucose level of 120 mg/dl results in selection of a median goal of 154 mg/dl, and a selection of a target glucose level of 140 mg/dl results in a median goal setting of 168 mg/dl.

A default target glucose level may be 120 mg/dl for most patients. However, for elderly patients or patients with fear of hypoglycemia may have a higher target glucose level, of for example 140 mg/dl. For patients who are more tolerant of hypoglycemia or who wish to maintain tight glucose control, a lower target glucose level setting of 110 mg/dl may be selected. The dose guidance system may select a corresponding median glucose goal for titration that is consistent with the patient's goals for the target glucose level selection and saves the HCP the difficulty of determining and setting an appropriate glucose median goal.

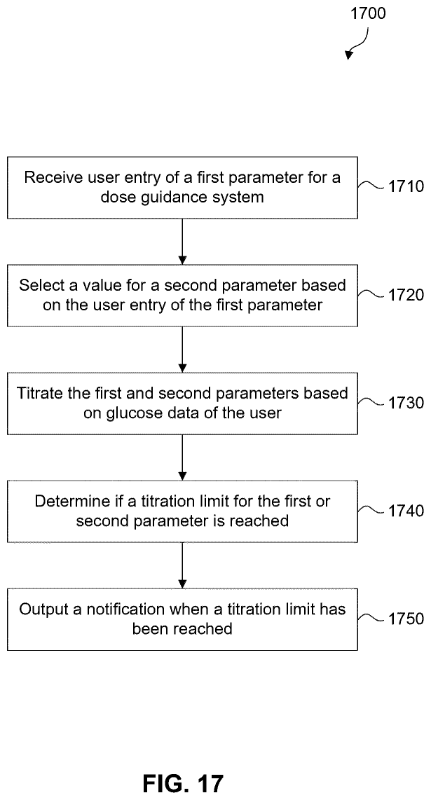

An exemplary method of configuring a dose guidance system is set forth in FIG. 17. A dose guidance system may receive user entry of a first parameter of a dose guidance system 1710. The dose guidance system may automatically select a value for a second parameter based on the input of the first parameter 1720. The system may separately titrate the first and second parameters based on glucose data of the user 1730. The system may determine if the titrated values of either the first or second parameters have reached a titration limit 1740. The system may output a notification that a titration limit has been reached 1750.

A dose guidance system may include a setting for a single correction factor, or for a plurality of correction factors for different times of day. The correction factor may include a pre-meal correction factor and a post-meal correction factor. While setting multiple correction factors may allow for more precise control of glucose levels, setting multiple correction factors or other parameters may be cumbersome for the user, and it may be difficult for the patient or HCP determine the appropriate values for the various parameters.

To simplify the set-up of such dose guidance systems, the system may use multiple correction factors but may prompt the user to configure a single correction factor. The system may set the correction factor to a default value that may be adjusted by the user (e.g., HCP). For example, the correction factor may be set to a default setting of 1:40 units per mg/dl. The system may prompt the user to confirm the default setting or may allow the user to adjust the default correction factor. The correction factor setting may be blank and may require the user to enter a value via a user input of the display device. The user may select a value for the correction factor from a list or may enter the value manually.

As it may be desirable to have more than one correction factor, the dose guidance system may prompt the user to enter a single correction factor, and may set additional correction factors based on the user entered correction factor. For example, the user may be prompted to enter a pre-meal correction factor, and based on the user entry of the pre-meal correction factor, the system may automatically set a value for a post-meal correction factor, or vice versa. In embodiments where the user selects the pre-meal correction factor, the post-meal correction factor may be determined based on the selected pre-meal correction factor based on Equation (2) as follows:

$$CFPost = α * CFPre \qquad ( 2 )$$

wherein CFₚₒₛₜ is the post-meal correction factor, CFₚᵣₑ is the pre-meal correction factor, and α is a ratio between the two values.

The pre-meal correction factor may be titrated by the dose guidance system as glucose data of the user is collected over time. The pre-meal correction factor may also be manually modified by the user after the initial setting of the pre-meal correction factor. The dose guidance system may update the post-meal correction factor according to Equation (2) as the pre-meal correction factor is titrated or manually adjusted.

Alternatively, the pre-meal correction factor and the post-meal correction factor may each be titrated separately by the dose guidance system, wherein Equation (2) is used to determine the initial setting of the post-meal correction factor based on the pre-meal correction factor. After the initial values for the pre- and post-meal correction factors are determined during initial set-up, the titration of the pre-meal correction factor may proceed independently of the titration of the post-meal correction factor.

In some embodiments, the post-meal correction factor may be titrated separately from the pre-meal correction factor, but may be limited by the value of the pre-meal correction factor. For example, the post-meal correction factor may be titrated independently of the pre-meal correction factor, but may not be set to a value below the value of the pre-meal correction factor.

In some embodiments, the pre-meal and post-meal correction factors are not configurable by the user. The pre-meal correction factor may be set to a default value, and the post-meal correction factor is set to a value based on Equation (2). In this way, the user does not have to enter any correction factor as part of set-up of the dose guidance system. The default value may be based on population data, and may be a conservative value. Titration of the correction factor by the dose guidance system over time will tailor the correction factor to the particular user.

Some embodiments described herein relate to titration of a correction factor. A correction factor may be titrated as described, for example, in US 2021/0050085A1, which is incorporated herein by reference in its entirety. The dose guidance system may titrate a pre-meal correction factor. The pre-meal correction factor may be titrated based on glucose data, such as glucose data collected by an in vivo glucose monitoring device. The glucose data segment for titrating the pre-meal correction factor, referred to as CFₚᵣₑ glucose data segments, may start with the first glucose level recorded following a meal dose and end at after a predetermined amount of time after the start time, e.g., 5 hours, or at the time of administration of a subsequent medication dose.

The CFₚᵣₑ glucose data segments may be assessed for validity prior to use in a titration determination, with invalid glucose data segments not being used in the titration determination. One or more of the following criteria may be used to determine validity of the CFₚᵣₑ glucose data segments. A CFₚᵣₑ glucose data segment may be deemed invalid if the duration is less than a predetermined duration, such as 4 hours. The CFₚᵣₑ glucose data segment may be deemed invalid if it was manually classified by the user (rather than automatically classified by the dose guidance system). The CFₚᵣₑ glucose data segments may be deemed invalid if a pre-dose glucose level is less than a predetermined low glucose level, such as 70 mg/dl. The CFₚᵣₑ glucose data segment may be deemed invalid if there is a gap in the glucose data longer a predetermined duration, such as 15 minutes, or 30 minutes, among other durations. The glucose data segment may be deemed invalid if associated with the last recorded meal dose (i.e., the most recently recorded meal dose or the most recently recorded meal dose to be considered for the titration analysis).

A pair of glucose values may be determined based on each CFₚᵣₑ glucose data segment for each meal dose. The pair of glucose values includes a pre-dose glucose level, and a post-dose glucose level. The pre-dose glucose level is the glucose level at the time the meal dose is administered. The post-dose glucose level may be determined to be a predetermined percentile glucose level in the CFₚᵣₑ glucose data segment. The predetermined percentile may be a 5^{th} percentile glucose level. However, it is understood that other percentiles may be selected. The 5^{th} percentile glucose level may be determined based on the glucose data in the CFₚᵣₑ glucose data segment between a maximum glucose level in the glucose data segment and a minimum glucose level after the maximum glucose level and before the end time of the glucose data segment.

The pairs of glucose values for each meal dose may be stored in memory, such as in a buffer. The buffer may retain the most recent 12 weeks of pre- and post-dose glucose data for a given pre-meal correction factor (CFₚᵣₑ) value. However, it is understood that other periods may be used, e.g., 10 weeks, 14 weeks. The pairs of glucose levels in the buffer can be used to perform a linear regression analysis to determine a slope and a p value. The results will determine if a new CFₚᵣₑ value should be suggested in the titration determination.

The dose guidance system may include a data sufficiency requirement to ensure the data used in the pre-meal correction factor titration determination is reliable. The buffer may include glucose data collected based on a previous value for CFₚᵣₑ and glucose data the current CFₚᵣₑ value. While all data in the buffer is used for the titration determination, the data sufficiency requirement may require the buffer to include pairs of glucose values collected at the current CFₚᵣₑ value over a minimum period of time, such as a minimum of 7 days.

The pairs of glucose values may be classified in one or more classes. The classes may include slow excursion, no excursion, or normal excursion. The classification may be based on one or more features of the CFₚᵣₑ glucose data segment. The features may include a height and a decrease of the CFₚᵣₑ glucose data segment. To compute the height and decrease, a maximum or “peak” glucose value in the CFₚᵣₑ glucose data segment is identified and divides the CFₚᵣₑ glucose data segment into a pre-peak segment and a post-peak segment. A minimum glucose value is then identified in each of the pre-peak and the post-peak segments. The height and decrease of the pre- and post-peak segments are the difference between the maximum glucose level and the pre-peak minimum and the post-peak minimum, respectively.

The glucose data segment is classified as a slow excursion if the decrease is greater than a threshold decrease, such as 30 mg/dl, and the ratio of decrease to height is less than a threshold, such as 60%. The CFₚᵣₑ glucose data segment is classified as no excursion if the glucose data segment has a height of zero. The CFₚᵣₑ glucose data segment is classified as a normal excursion if the glucose data segment is not classified as slow excursion or no excursion.

The pairs of glucose values of the CFₚᵣₑ glucose data segments are grouped according to the associated meal (e.g., breakfast, lunch, or dinner) and based on the classification (slow excursion, no excursion, or normal excursion). Thus, there may be 9 groups: breakfast-slow excursion, breakfast-no excursion, breakfast-normal excursion, lunch-slow excursion, lunch-no excursion, lunch-normal excursion, dinner-slow excursion, dinner-no excursion, and dinner-normal excursion. For each group, a mean of the pre-dose glucose level and a post-dose glucose level of each pair of glucose values is computed. For each pair in the group, the mean is subtracted from each of the pre- and post-dose glucose values to determined updated glucose pairs. The updated glucose pairs from the groups that contain at least two unique correction adjusted values are included in the linear regression analysis, and updated glucose pairs with one unique correction adjusted value (rounded to the nearest integer) will be excluded from the linear regression analysis. The correction adjusted value is determining by dividing the difference between the pre-meal glucose and the target glucose by CFₚᵣₑ. After the adjusted glucose pairs of all groups are combined together, at least a minimum number of unique correction adjusted values are needed to perform the linear regression analysis, such as two or more. If there are two, a minimum of 3 pairs of glucose values for each of the two correction adjusted values is needed for the linear regression analysis. If such requirements are not met, the pre-meal correction value will be maintained at the current value.

Titration of the pre-meal correction factor may proceed in a first mode or a second mode. The first mode may be a “rapid mode” and the second mode may be a “stable mode.” When the dose guidance system is first used, titration of the pre-meal correction factor proceeds in first mode until transition logic is satisfied. The pre-meal titration output is determined by the slope of the linear regression. If the slope is greater than zero, the value of the pre-meal correction factor is decreased. If the slope is zero or negative, the pre-meal correction factor is increased.

The pre-meal correction factor titration transitions from the first mode to the second mode upon fulfillment of transition criteria. The transition criteria may involve analysis of the pre-meal correction factor values. After each titration, the new or updated pre-meal correction factor is stored in memory, such as in a buffer. If the pre-meal correction factor oscillates between a first value and a second value, the pre-meal correction factor titration may transition from the first mode to the second mode. The oscillation may be determined based on a shift between two correction factor values over a predetermined number of titrations, such as 5 titrations. The transition criteria may further require the correction factor index to be less than a predetermined value, such as 13. The CFₚᵣₑ and CFₚₒₛₜ values may be selected from a list of predefined values represented by a correction factor index (CFI). For example, there may be 15 possible values of CFₚᵣₑ and CFₚₒₛₜ and accordingly the CFI may be 1 to 15, with each number representing a correction factor value. The CFI is determined by comparing the initial pre-meal correction factor value with the list of pre-defined pre-meal correction factor values. If the pre-meal correction factor is determined to be increased, CFI=previous correction factor+1, and when the pre-meal correction factor is decreased, CFI=max (3, previous correction factor−1).

When the transition criteria are met, the pre-meal correction factor titration transitions to the second mode or stable mode. Similar to the first mode, the pre-meal titration output is determined by the slope of the linear regression. If the slope is greater than zero, the value of the pre-meal correction factor is decreased. If the slope is zero or negative, the pre-meal correction factor is increased. However, in the second mode, the value of the correction factor is only changed if the p-value of the linear regression is less than a threshold p-value, such as 0.05.

The recommended pre-meal correction factor as determined based on the titration determination described herein may be presented to the user for approval. When the user approves the recommended pre-meal correction factor, the pairs of glucose values that were used for the current titration continue to be used for future titrations subject to the validation and data sufficiency requirements described above.

The buffer of pairs of glucose values may be cleared if the user manually reduces a dose or enters a new correction factor. The pre-meal titration algorithm may transition back to the first mode (rapid mode). The titration analysis proceeds with glucose values collected after the manual dose reduction.

Some embodiments described herein relate to titration of a post-meal correction factor. The post-meal correction factor may be titrated using glucose pattern analysis as described herein. The dose guidance system may analyze glucose data following each post-meal correction dose, referred to as a CFₚₒₛₜ glucose data segment. The post-meal correction dose may be automatically classified as a post-meal correction dose by dose guidance system. The CFₚₒₛₜ glucose data analyzed may be glucose data in a predetermined time period following the correction dose. The CFₚₒₛₜ glucose data segment may have a start time that is one hour after administration of the post-meal correction dose, and have an end time after a predetermined amount of time following the start time of the CFₚₒₛₜ glucose data segment, such as 4 hours. Alternately, the end time may be the start time of a next insulin dose. The dose guidance system may detect low glucose events or low glucose alarms that occur during the CFₚₒₛₜ glucose data segment.

The CFₚₒₛₜ glucose data segments may be checked by the dose guidance system for validity prior to being used in a titration determination. A CFₚₒₛₜ glucose data segment may be deemed invalid if there are gaps in the glucose data greater than a predetermined threshold, such as 30 minutes or more. A CFₚₒₛₜ glucose data segment may be deemed invalid if the CFₚₒₛₜ data segment has a duration less than a predetermined duration, such as 3 hours. The CFₚₒₛₜ glucose data segments are invalid if not recorded within a predetermined period of time before the titration determination, such as 12 weeks.

The post-meal correction factor may be titrated based on a most recent set of CFₚₒₛₜ data segments. The most recent set may include the most recent 14 CFₚₒₛₜ data segments. Relying on a limited number of most recent glucose data segment helps to improve accuracy while avoiding delay of titration and titrating based on sufficiently recent data. Once 14 glucose data segments are collected, the oldest valid data segment is deleted as a new glucose data segment is recorded. The titration determination may require a minimum amount of glucose data segments, and may require a minimum of 7 CFₚₒₛₜ glucose data segments.

The titration of the post-meal correction factor may be based on the valid CFₚₒₛₜ glucose data segments. The valid CFₚₒₛₜ glucose data segments are subject to the glucose pattern analysis as described herein. The glucose data segments may be treated as belonging to a breakfast TOD period. If the glucose pattern results in a High pattern, but there are any glucose levels in valid data segments below a low glucose level, such as 70 mg/dl, the assessed pattern is changed to a No pattern. If the number of low glucose events or low glucose alarms is greater than a predetermined number, then the pattern is changed to a Low pattern regardless of the original pattern determination.

The titration determination includes increasing the CFₚₒₛₜ if the Low pattern is assessed. The titration decision includes decreasing the CFₚₒₛₜ if the glucose pattern is a High pattern. The increase or decrease of the CFI may be by a predetermined amount, such as 1. If any other pattern is detected, the titration determination is to maintain the CFₚₒₛₜ. A minimum CF value may be assessed, such that if the titration decision to decrease CFₚₒₛₜ, CFₚₒₛₜ may not be reduced below the minimum CF value. If CFₚₒₛₜ is titrated along with the fixed-dose titration of meal doses, and the glucose pattern for any other TOD period is a Low pattern, the current CFₚₒₛₜ value is maintained.

The default values for the parameters of the dose guidance system may be selected based on user characteristics. The user's characteristics may include the patient's body weight, age, BMI, or gender, and combinations thereof, among other user characteristics. The dose guidance system may prompt the user to enter one or more user characteristics, such as via an input of a display device, and may use the entered user characteristics to set default values for parameters of the dose guidance system. The default values for the parameters may be based on population data such that the default parameter values are based on the parameters used for patients in the population having similar characteristics. For example, the default setting for the pre-meal correction factor for a user may be based on the pre-meal correction factor for other users in the same age range and/or weight range. Setting the parameters based on user characteristics further helps to alleviate the burden of the user having to select appropriate values for the parameters, such as pre-meal and post-meal correction factors which may be difficult to determine without guidance.

The dose guidance system may allow for entry of a titration limit relating to a total amount of medication that can be recommended by the system. The limit may be a maximum total daily dose (TDD). This limit may help to prevent the system from administering too much insulin to the patient, which can result in hypoglycemia, and to ensure that the HCP is involved in the adjustment of the patient's therapy. TDD may be a sum of all insulin doses administered in one day. For example, the TDD may be the sum of a basal dose, breakfast dose, lunch dose, and dinner dose. Alternatively or additionally, the titration limit may include a limit for each medication dose. For example, the basal dose may have a maximum allowable basal dose setting, the breakfast dose may have a maximum allowable dose setting, the lunch dose may have a maximum allowable dose setting, and the dinner dose may have a maximum allowable dose setting. Setting a TDD simplifies set-up of the dose guidance system and may be easier for the HCP to determine rather than setting limits for each individual medication dose. The TDD limit may be an absolute value or may be based on an allowed percentage increase relative to the current dose or most recent dose. For example, the maximum dose may be 25% greater than the current dose, 50% greater than the current dose, or 100% greater than the current dose.

The dose guidance system may titrate the parameters, such as the medication doses, until the titrated value reaches or exceeds a titration limit, in which case the dose guidance algorithm will not recommend an adjustment that exceeds the titration limit. If the dose guidance algorithm determines a dose that reaches or exceeds the titration limit, the system may output a notification to the user, such as on a display device of the user, indicating that the user should contact their HCP and consider increasing the titration limit. A notification may alternatively or additionally be communicated by the dose guidance system to a computing device of the HCP indicating that a parameter of the dose guidance system, such as a TDD, has reached the titration limit and requesting the HCP to increase the titration limit. Further, a glucose report including glucose data and/or medication data over a preceding period of time, such as 7 days, may be displayed along with a recommendation to adjust the titration limit.

A titration limit may include a minimum value for a parameter. For example, there may be a setting for a minimum value for a correction factor. The minimum value for the correction factor may be automatically selected by the system based on the limit manually set by the user for the TDD. For example, if the maximum increase in the TDD is 25%, the minimum value for the correction factor may be set to a value that is 25% less than the current correction factor setting. For example, a user may be requested to select a limit for the TDD. Once the limit for the TDD is set, the system automatically selects the minimum CF setting based on the limit set for the TDD. This may help to further simplify the initial configuration of settings for the user. Further, as discussed, the TDD may be a more easily understood value for the user to set, whereas the correction factor setting may be more difficult for the user to determine.

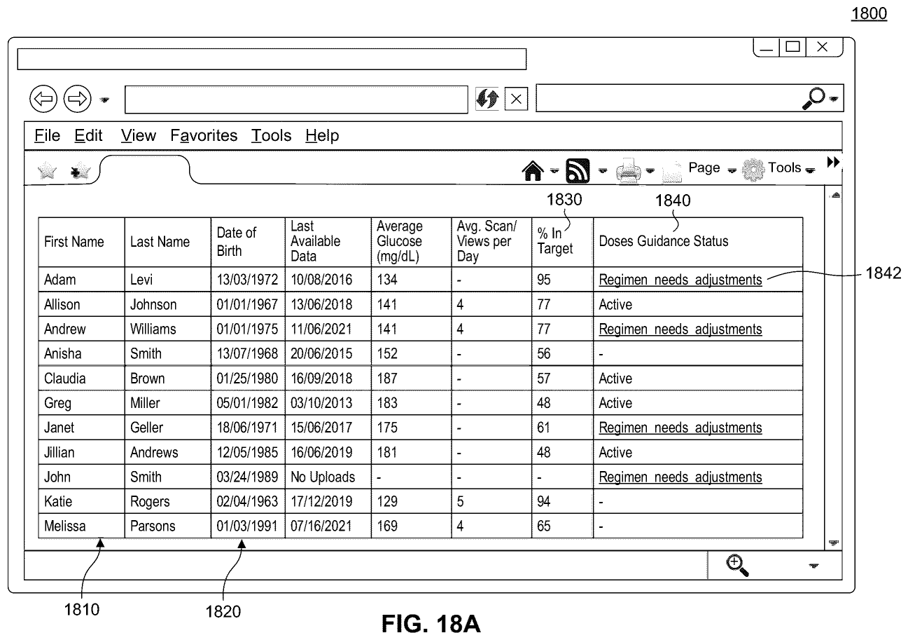

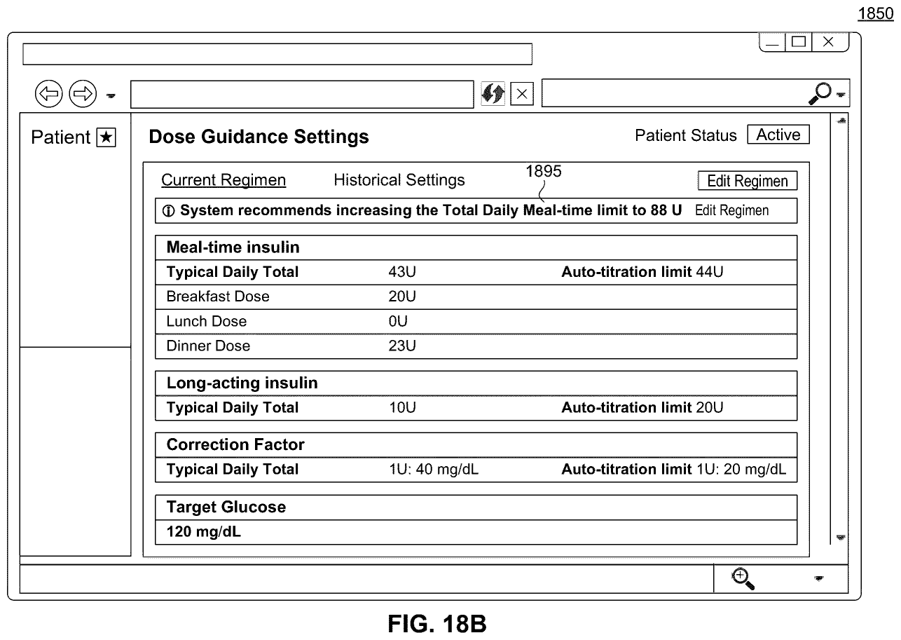

An exemplary user interface for a HCP is shown in FIG. 18A. The HCP device may include an interface 1800 showing a list of the HCP's patients. The interface 1800 may include a row for each patient and columns providing patient information for the particular patient. However, it is understood that the patient list and patient information may be displayed in other formats. The patient information may include the patient name 1810. The patient information includes personal information 1820 such as the patient's age, weight, or gender, among other information. The patient information includes glucose data 1830, such as one or more glucose metrics. The glucose metrics may be indicative of the user's glycemic control, such as average glucose, median glucose, or time in range (TIR), among others. The glucose metrics may indicate the user's use or compliance with use of the system, such as an average number of scans per day, an average number of views of glucose data per day, or information relating to the amount of time the user wears a glucose monitoring sensor.

Interface 1800 may further include a dose guidance status 1842. The dose guidance status 1842 may alert the HCP that the user's dose guidance settings may need to be adjusted. The dose guidance status 1842 may be selected from: needs adjustment, active (no adjustment needed), or none. For example, if titration of one or more of the parameters of the dose guidance system has reached a titration limit, interface 1800 may provide a notification in dose guidance status 1842 to indicate that adjustment of the user's dose guidance settings may be needed. For example, if titration has progressed such as a maximum TDD has been met or exceeded. The dose guidance status 1842 may indicate to the HCP that the dose guidance system is active (and no adjustments are needed), or that the dose guidance system is not available or is otherwise inactive (e.g., for patients who do not use a dose guidance system). The interface 1800 and dose guidance status 1842 helps the HCP to easily monitor the progress of the medication titration for multiple patients, and to easily identify which patients may require an adjustment to their therapy.

Interface 1800 may allow the HCP to adjust user dose guidance settings, and may recommend the adjusted dose guidance settings for the HCP to confirm or modify. For example, the dose guidance status 1842 may be selected by the HCP to re-direct the HCP to interface 1850 as shown in FIG. 18B. Interface 1850 in FIG. 18B is the same as interface 1600 of FIG. 16 but includes a notification 1895. The notification may include a recommended change to the parameters. For example, as shown in FIG. 18B, notification 1895 includes a recommendation that the user edit one or more dose guidance parameters. The notification 1895 may include the parameter to be adjusted. The notification 1895 may include a recommended new value for that parameter. For example, in FIG. 18B, the notification 1895 alerts the user to increase the TDD for meal-time insulin to 88 U.

If the HCP modifies the parameters of the dose guidance system via interface 1600, the recommended new setting may be communicated to the display device of the user. The updated value for the parameter may be automatically go into effect. The dose guidance system may provide a notification to the user, such as on the display device of the user, that a dose guidance parameter has been modified. The dose guidance parameter may automatically go into effect or may require user confirmation of the change of the parameter before going into effect.

As discussed above, the dose guidance system requires input from an HCP to determine the appropriate settings for the parameters of the dose guidance system. However, the dose guidance application is typically provided on a display device of a user, such as on the user's smartphone, for use by a user on a day-to-day basis. Thus, there is a need to help confirm and ensure that the HCP is involved in the initial set-up and configuration of the dose guidance system.

In order to ensure an HCP is involved in the set-up of the dose guidance system, the system may request verification that the user setting the initial configuration of the dose guidance system is an HCP. The user interface may prompt a user to enter a code, wherein the code is only available to HCPs. The system compares the entered code to a database of acceptable codes to determine if the entered code is valid. The system enables use of the dose guidance system if the entered code is valid. If the code is not valid, the user is unable to use the dose guidance system to generate recommended medication doses. The user may manually enter a code, such as using an input device to enter text, such as a numerical code, an alphabetical code, or an alphanumeric code, or the like. Alternatively, the user may scan a code, such as a barcode or QR code. The user may scan the code using a camera of the display device, e.g., smartphone, on which the dose guidance application is installed. The QR code may encode the authentication code to verify that the user is an HCP or is accompanied by an HCP.

The dose guidance system may provide a code, such as an alphanumeric code to the user. The user may then access an application or webpage on a computing device, such as a laptop, computer, or the like, wherein the application or webpage is only accessible to HCPs. Once the code is entered into the application or webpage, the system would communicate with the dose guidance application on the display device of the user to allow the user to proceed to use the dose guidance system.

Some embodiments described herein relate to a dose guidance system configured to provide a late dose alert to notify a user that the user has not taken a dose of medication. This may help the user to stay compliant with the user's medication therapy regimen, and avoid loss of glucose control and delay of titration that may result from missing a medication dose. Patients may periodically forget to take a scheduled dose of medication. Missing a dose may result in poor glycemic control and increase the risk of the user experiencing hyperglycemia and other negative health consequences. Further, as discussed herein, missing a dose may delay titration as the algorithm may require a minimum amount of valid TOD periods and a minimum amount of data in each TOD period to recommend an adjusted dose.

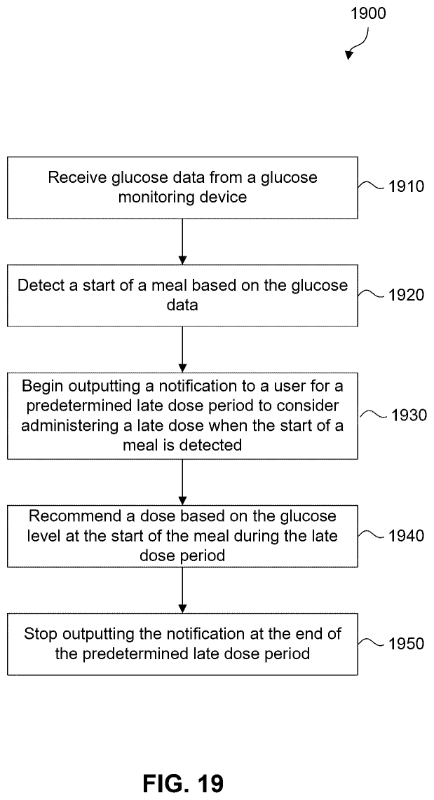

In an exemplary method of providing a late medication dose notification as shown in FIG. 19, the system receives glucose data from a glucose monitoring device 1910. The system detects if a meal has started based on glucose data 1920. The start of a meal may be determined based on analysis of one or more glucose metrics, based on historical glucose data for the user indicative of the user's response to previous meals, and/or based on population data indicating the glucose response to meals of a population of users. The glucose data may be input to a model, such as a machine learning model, configured to identify a pattern of glucose data indicative of a meal. The model may be trained on population data or historical glucose data of the user. Machine learning models can include, by way of example and not limitation, models trained using or encompassing decision tree analysis, gradient boosting, adaptive boosting, artificial neural networks or variants thereof, linear discriminant analysis, nearest neighbor analysis, support vector machines, supervised or unsupervised classification, and others. The models can also include algorithmic or rules-based models in addition to machine learning models. In alternate embodiments, the meal may be detected by one or more of the methods described herein, or may be determined based on a combination of one or more methods described herein, and may be determined in combination with other methods known in the art for detecting a meal as would be understood by one skilled in the art.

If the system detects a meal has started, the system outputs a notification to the user for a late dose period 1930. The notification may alert the user that the user may have missed a medication dose. The notification may recommend that the user administer a late dose of medication. The notification may be a pop-up window on the user interface, a banner type notification, or may be an icon or other indicator displayed on the user interface of a glucose monitoring application or dose guidance application (e.g., on a mobile phone or a smart watch). The system may output a notification based solely on the glucose data. Alternatively, the system may output a notification only if no dose has been recorded as being administered by the user for the meal. Medication administration information may be manually entered by the user or may automatically captured by a medication delivery device or dose monitoring device.

The system may recommend a dose amount (e.g., a fast-acting insulin dose) that is calculated based on a glucose level at the start time of the detected meal 1940. The system may stop outputting or stop presenting the notification after the predetermined late dose period 1950. The predetermined late dose period may be, for example, in a range of 1 hour to 3 hours. The late dose period may be 2 hours. The notification may similarly stop outputting or presenting if the user enters an input to decline a recommended late dose, or upon detection or confirmation of administration of a medication dose. Administration of a medication dose may be determined based on user input indicating a medication dose has been administered, automatic capture of the dose by a medication delivery device or dose monitoring device, or based on glucose data indicative of administration of a medication dose (e.g., a pattern of declining glucose levels).

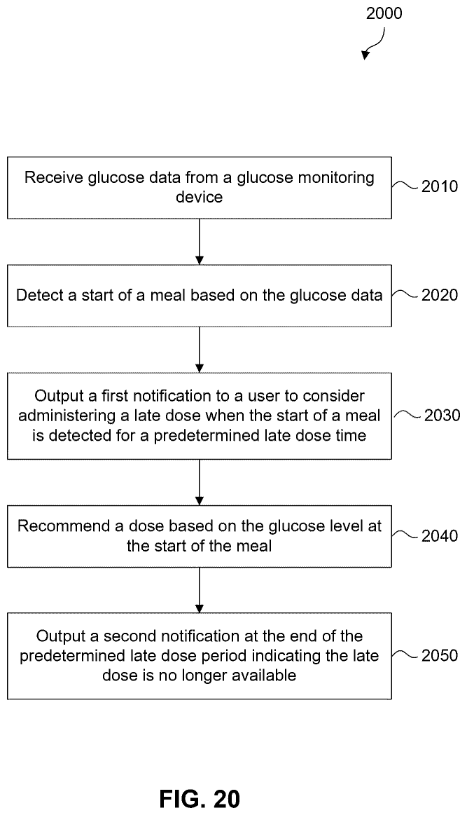

In another exemplary embodiment as shown in FIG. 20, the system receives real-time glucose data from a glucose monitoring device 2010. The system detects if a meal has started based on glucose data 2020. If the system detects a meal has started, the system outputs a first notification to the user 2030. The notification may alert the user that the user may have missed a medication dose. The notification may recommend that the user administer a late dose of medication. The system may recommend a dose amount that is calculated based on a glucose level at the start time of the detected meal 2040. The system may output a second notification at the end of the late dose period 2050. The second notification may indicate that the late dose should no longer be administered or that the late dose is no longer available. This may help to prevent the user from taking the recommended dose too long after the meal and when the user's glucose levels have changed from the time when the dose was determined.

The dose guidance system may include a setting to enable or disable late dose notifications. The dose guidance system may alternatively or additionally include a setting to enable or disable correction dose notifications. Correction dose notifications may provide a recommendation to the user to administer a correction dose, such as when high glucose levels are detected. The setting of the late dose notification may automatically set the correction dose notification setting. For example, if the late dose notification setting is enabled, the correction dose notification may automatically be set to enabled. This helps to relieve the user from individually setting the various parameters of the system by tying together related settings.

Some embodiments described herein relate to setting initial parameters of the dose guidance system based on the user's data collected during a learning period. The learning period may be a predetermined period of time prior to initiation of the dose guidance system. For example, the learning period may be 7 days, 10 days, or 14 days, among others. During the learning period, the system may receive glucose data from a glucose monitoring device. The system may further receive medication administration information. The medication information may include one or more of the type of medication, e.g., long-acting insulin or rapid-acting insulin, medication administration times, or dose amounts, among other information. The medication information may be manually entered by the user or automatically captured by a medication delivery device or dose capture device.

The data from the learning period may be used to configure initial dose regimen parameters, such as one or more of a basal dose amount, one or more meal-time dose amounts, one or more correction factors, a target glucose level, or mealtimes, among other parameters.

The dose guidance system may use a machine learning model that receives the glucose data and medication data from the learning period as inputs, and the machine learning model is configured to output a set of dose amounts. Machine learning models can include, by way of example and not limitation, models trained using or encompassing decision tree analysis, gradient boosting, adaptive boosting, artificial neural networks or variants thereof, linear discriminant analysis, nearest neighbor analysis, support vector machines, supervised or unsupervised classification, and others as described herein. The dose amounts may include one or more of a basal dose amount and meal or rapid-acting dose amounts. The machine learning model may alternatively or additionally determine and output a correction factor. A correction factor may be estimated by the dose guidance system by determining a TDD based on the medication data collected during the learning period, and dividing the TDD by a fixed value, e.g., 1700 U/mg/dl.

To determine the dose classification of the rapid-acting doses administered during the learning period, the model may use a clustering analysis. A clustering analysis is disclosed, for example, in U.S. Publication No. 2021/0050085A1, incorporated herein by reference in its entirety. The clustering analysis may implement a K-mean algorithm together with the elbow method. The clustering analysis uses dose administration times as the input and outputs the optimal number of clusters, K, wherein K is at least 1 and is no greater than 3, and outputs the cluster index for each dose administration time. The optimal number of clusters K can be the number of meal doses taken by the patient per day. The dose guidance system can group the rapid-acting doses into K clusters according to the cluster indexes. The dose guidance system can identify the groups as, for example, breakfast, lunch, or dinner.

The optimal number of clusters, K, may be determined by assessing if each of a list of criteria is true for a particular value of K, starting with K=3, and then K=2, and then K=1. If each criterion is true for a particular K value, then K is set to that value. The criteria may include one or more of: no clusters have fewer than a predetermined number of data points (e.g., no fewer than 4 data points), the centers of each cluster are at least a predetermined number of hours apart (e.g., at least 3 hours apart), and/or a center of a last cluster and a center of the first cluster is at least a second predetermined number of hours apart (e.g., 9 hours). The center of the cluster may be determined based on a median of all values in each cluster. Alternately, the center of the cluster may be determined by other measures of central tendency, such as an average value.

Once the rapid-acting or meal doses are classified (e.g., breakfast doses, lunch doses, dinner doses), such as by using a model with a clustering analysis as described above, the model extracts glucose features from the glucose data following each meal, i.e., post-prandial glucose data. The glucose features may include one or more of mean glucose, glucose variability, area under the curve on a plot of glucose levels over time, 5^{th} percentile of glucose data, 95th percentile of glucose data, time in range, time above range (e.g., time above 180 mg/dl), or time below range (time below 70 mg/dl). For a given set of glucose features for a meal, a regression estimate is used to estimate an increase or decrease of the corresponding meal dose. The increase or decrease may be expressed as a percentage relative to the current meal dose. The percent change in the meal dose is optimized to maximize a glucose feature that measures or is indicative of glucose control, such as time in range, among others. The percent change in meal dose may further be optimized to reduce or minimize a glucose feature indicative of hypoglycemia, such as time below range or 5^{th} percentile glucose data. The percent change in meal dose may be optimized to reduce a glucose feature that is indicative of hyperglycemia, such as 95th percentile glucose data, or time above range. The dose guidance system may have a goal to optimize the medication doses to increase the user's time in range while minimizing incidences of hypoglycemia.

A basal dose amount may be determined in a similar manner as described above with respect to the rapid-acting doses. However, rather than using glucose data from a post-prandial period, the model may extract glucose features from glucose data following administration of each basal dose, such as in a 24-hour period following administration of the basal dose. The glucose features may include one or more of mean glucose, glucose variability, area under the curve on a plot of glucose levels over time, 5^{th} percentile of glucose data, 95th percentile of glucose data, time in range, time above range (e.g., time above 180 mg/dl), or time below range (time below 70 mg/dl). The model may use a regression estimate to determine whether to increase or decrease the basal dose. The model may determine a dose amount that optimizes one or more of the glucose features. The increase or decrease may be expressed as a percentage relative to the current basal dose. The percent change in the basal dose is optimized to maximize a glucose metric that is indicative of glucose control, such as time in range, among others. The percent change in meal dose may further be optimized to reduce or minimize a hypoglycemia metric. In this way, the basal dose may be optimized to increase time in range while minimizing incidences of hypoglycemia.

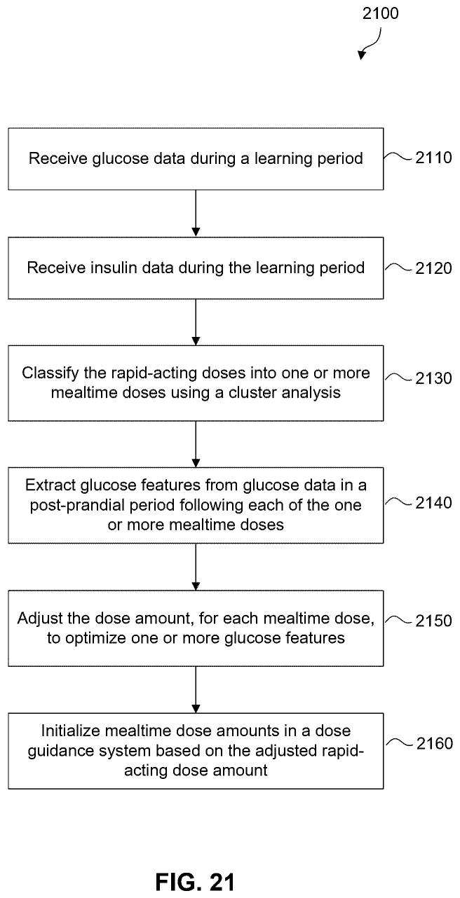

An exemplary method of initializing dose amounts for a dose guidance system based on data collected during a learning period is shown in FIG. 21. The dose guidance system receives glucose data collected during a learning period 2110. The glucose data may be received by a glucose monitoring device which may be in communication with a display device of the user. The dose guidance system receives insulin data during the learning period 2120. The insulin data may include dose amounts, dose times, and medication type, such as long-acting insulin or rapid-acting insulin. The rapid-acting doses are classified into one or more meal doses using a clustering analysis 2130. As the user may not administer insulin doses at the exact same time each day, the clustering analysis is used to associate each rapid-acting dose with a particular meal, such as one or more of breakfast doses, lunch doses, and dinner doses. Glucose features are extracted from the glucose data in a post-prandial period following each mealtime dose 2140. The post-prandial period may have a start time that is a center of a cluster, e.g., a median dose time of doses in the cluster. The post-prandial period may be deemed to end after a fixed amount of time after the start time, e.g., 5 hours. Alternatively, the post-prandial period may end at a start time of a subsequent meal dose. The meal doses administered by the user during the learning period may be adjusted to optimize one or more of the glucose features 2150. The meal doses may be set to initial values based on the adjusted dose amounts 2160.

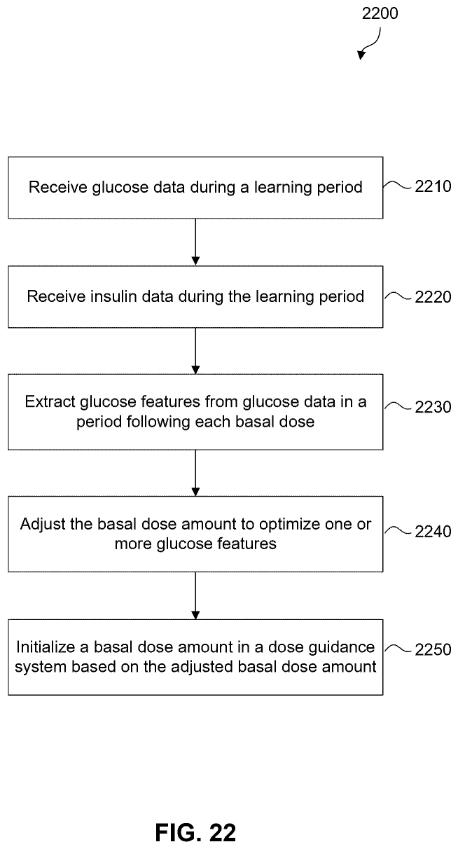

An exemplary method of setting an initial value for a basal insulin dose in a dose guidance system 2200 is shown in FIG. 22. A basal dose amount for the dose guidance system may be set to an initial value based on the data collecting during the learning period. The basal dose may be determined in a similar manner as discussed above for the rapid-acting or meal dose amounts, but does not require a cluster analysis, and instead the glucose features are extracted from glucose data in a period of time following administration of the basal dose.

The dose guidance system receives glucose data collected during a learning period 2210. The glucose data may be received by a glucose monitoring device which may be in communication with a display device of the user. The dose guidance system receives insulin data during the learning period 2220. The insulin data may include dose amounts, dose times, and medication type, such as basal dose (i.e., long-acting insulin) or rapid-acting insulin doses. Glucose features are extracted from the glucose data in a period following each basal dose 2230. The period following each basal dose may have a start time that is a median time of administration of the basal doses during the learning period. The period following each basal dose may end after a fixed amount of time following the start time, such as 24 hours. The basal dose amount may be adjusted to optimize the one or more glucose features 2240 in the period following the basal dose. The basal dose in the dose guidance system may be set to an initial value based on the adjusted basal dose 2250.

Some embodiments described herein relate to a dose guidance system for titrating a basal dose of insulin. A basal dose of insulin may be titrated over time based on the user's glucose data to achieve glycemic goals, such as improving time in range and reducing instances of hypoglycemia. When the glucose data indicates that the basal dose should be increased, the basal dose may be increased by a fixed amount (e.g., increase in increments of 2 units), or by a percentage of a TDD (e.g., increase the basal dose by an amount that is 10% of the TDD). However, if the user's glucose levels are far from a target level, such as when the user's glucose levels are very high, the basal dose may ultimately need to be increased by a relatively large amount to achieve full titration. Thus, titrating the basal dose by gradually increasing the dose in relatively small increments may delay the titration process and slow achieving glucose control.

Further, HCPs may not have the necessary experience and training to review continuous glucose monitoring data and to determine a specific adjustment to the basal dose. Dose guidance systems may help to close the knowledge gap by recommending the amount of the adjustment or the recommended dose amount.

In some embodiments, the dose guidance system may titrate the basal dose based on analysis of glucose monitoring data. The dose guidance system segments the glucose data into a plurality of time bins. The glucose data can be segmented into bins corresponding to TOD periods, as described herein, such as a breakfast period (or “bin”), a lunch period, a dinner period, and an overnight period. Alternatively, the glucose data may be segmented into bins of a fixed number of hours, such as 2-hour bins. It is understood that bins of other amounts of time may be used, such as 1 hour bins, 3 hour bins, etc.

The dose guidance system may recommend a medication dose based on determination of an optimal dose. The recommended dose may be less than the optimal dose, and may be determined as a percentage of the optimal dose, so that the optimal dose is approached in multiple steps. This may help to avoid incidences of hypoglycemia resulting from a large dose increase from the current dose to the determined optimal dose. The optimal dose may be determined based in part on a comparison of the user's glucose median to a goal median.

In some embodiments, the optimal dose may be calculated by Equation (3) as follows:

$$optimal dose = current dose + \frac{current median - goal median}{median sensitivity} \qquad ( 3 )$$

wherein the goal median is a goal glucose median value that is the lowest glucose median with minimal hypoglycemia the patient can achieve given the glucose variability of the patient. In some embodiments, goal median may be calculated by Equation (4) as follows:

$$Goal Median = Current Median - Margin \qquad ( 4 )$$

The Margin is defined as the difference between a low percentile of glucose data and a hypoglycemia threshold. The hypoglycemia threshold may be a low glucose level, for example, 70 mg/dl. However, it is understood that other hypoglycemia threshold values may be selected, e.g., 75 mg/dl, or 60 mg/dl, among others. The low percentile may represent a maximum acceptable amount of hypoglycemia. The low percentile may be a 4^{th} percentile of glucose data over a predetermined number of days of glucose data. The Margin is expressed in units of mg/dl (or mmol/l). The difference may be taken from the lowest value of the 4^{th} percentile of glucose data in a particular TOD period or bin. For example, the Margin may be determined based on the difference between the 4^{th} percentile glucose value and the hypoglycemia threshold in an overnight period. The Margin may be a minimum margin, a maximum margin, or an average margin.

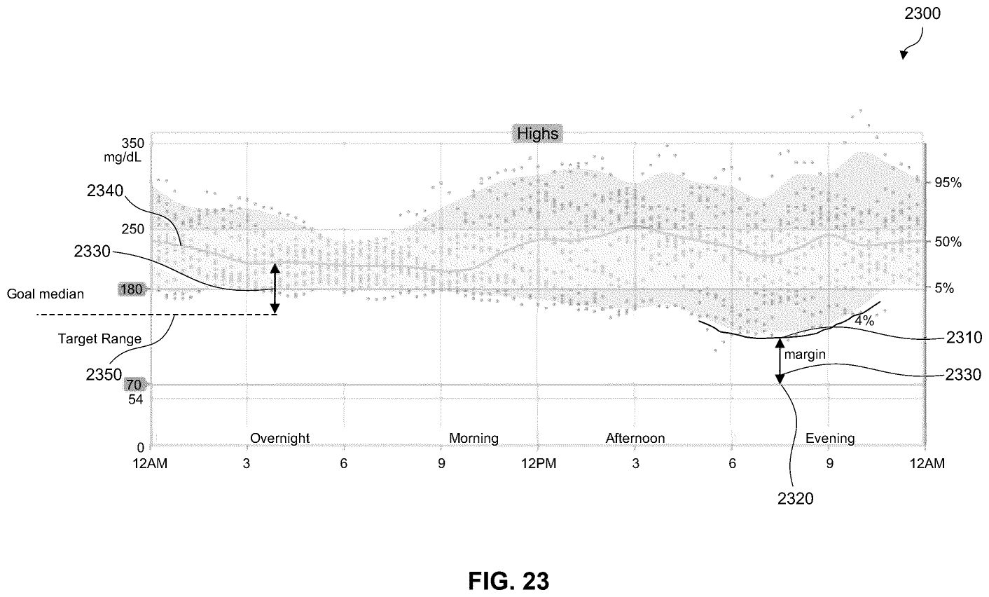

FIG. 23 shows a graphical representation of the margin and goal median. FIG. 23 shows a plot 2300 of different percentiles of the user's glucose data over a 24-hour period based on glucose data collected over multiple days. The margin 2330 is determined as the difference between the 4^{th} percentile glucose value 2310 and a hypoglycemia threshold 2320. The 4^{th} percentile glucose value 2310 may be taken as the lowest value in a TOD period, such as in the overnight period. The Goal Median 2350 may be determined based on the current median 2340 minus the Margin 2330. The current median may be determined as the average glucose median in a TOD period, such as in the overnight period.

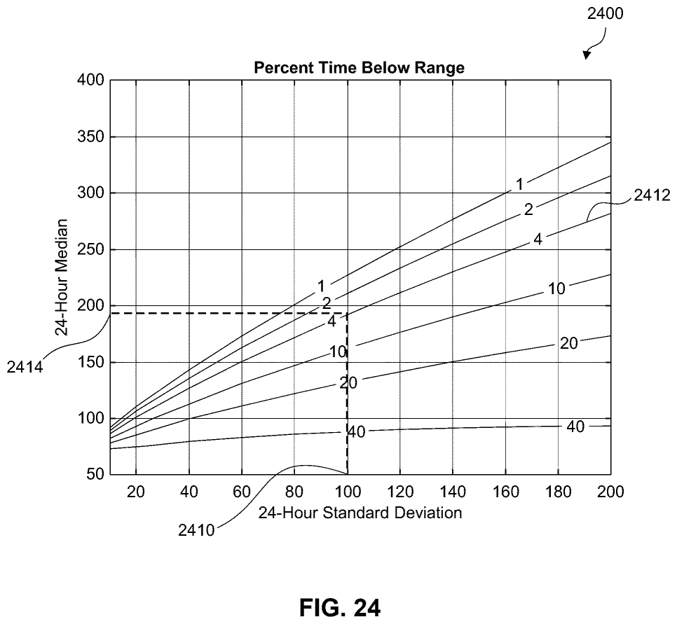

In another embodiment, Goal Median may be calculated based on a measure of glucose variability for the user and a time below range value. The time below range value may be set by the dose guidance system to a value that is a maximum allowable time below range. The time below range may be 4%. However, it is understood that other time below range values may be used, such as 2% or 10%, among others. The glucose variability may be a standard deviation of the user's glucose data over a period of time, such as over a 24 hour period. As shown for example in FIG. 24, different percentage time below range values are plotted on a graph of glucose median over standard deviation 2400. If the user's standard deviation of glucose values 2410 is 100 mg/dl, and the maximum time below range 2412 is selected to be 4%, then the Goal Median 2414 for the user is 190 mg/dl.

The median sensitivity in Equation (3) may be set to a default or initial value at the start of titration. The median sensitivity may be in a range of 0.2 to 2 mg/dl/U, 0.4 to 1.8 mg/dl/U, or 0.6 to 1.5 mg/dl/U. The median sensitivity may be based on population data on median sensitivity from in-silico simulations, based on median sensitivity data for other users, or from published studies. The median sensitivity can be calculated for a particular user once glucose data corresponding to two or more basal doses taken by the user is available. In some examples, median sensitivity based on population data or other users may be used until glucose data corresponding to two or more basal doses taken by the user is available, at which point the median sensitivity for the particular user may be used. The dose guidance system may determine the overnight median glucose value associated with a basal dose of insulin. The plot of points of basal dose and overnight glucose median may be constructed and a fit line for the points is generated. The median sensitivity can be determined as the slope of the fit line. Additional points may be added to the plot as the user continues to administer basal doses, and the fit line may be adjusted accordingly.

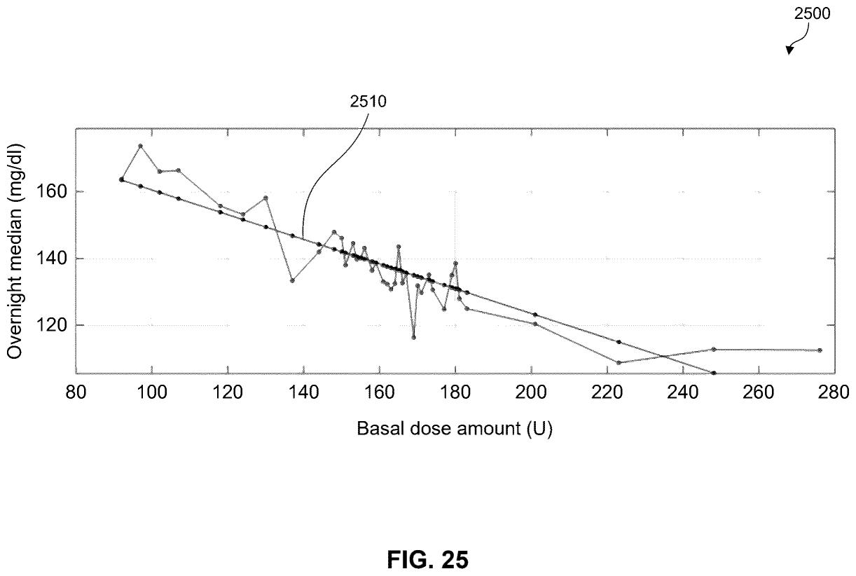

An exemplary plot of the TOD glucose median over the basal dose amount 2500 is shown in FIG. 25. A linear fit of the glucose median in a TOD (e.g., overnight period) to the basal dose can be determined, and the median sensitivity is the slope of the linear fit line 2510. Although the slope is negative in the plot, the median sensitivity is expressed as a positive value.

Accordingly, an optimal basal dose can be determined based on the current median, goal median, and median sensitivity as discussed above. The amount of a recommended change to the user's basal dose may be determined based on a difference between the current dose and the optimal dose. The recommended dose change may be a percentage or fraction of the difference of the current and optimal doses. The dose change may be determined by Equation (5) as follows:

$$Dose change = α \times ( optimal dose - current dose ) \qquad ( 5 )$$

wherein α is a safety factor, and is 0<α<1. By having a safety factor less than one, the dose guidance system approaches the optimal dose in more than one step to avoid increasing incidences of hypoglycemia. The safety factor can be set to a higher value when a larger dose change is desirable and set to a lower value when a smaller dose change is desirable. The amount of dose change may be based on a hypoglycemia risk metric. The hypoglycemia risk metric may be based on Gᵣᵢₛₖ, as described herein. Alternately, the hypoglycemia risk metric may be based on the Margin. The safety factor may also depend on the median sensitivity value and may be set to a lower value when the median sensitivity is aggressive and vice versa. The safety factor may be determined based on the confidence interval or p-value of the straight line fit such that the safety factor is set to a lower value if the confidence of the linear fit is lower, and can be set to a higher value when the confidence of the linear fit is higher.

Based on the above equations, the recommended dose change from the current dose may alternately be determined based on the margin and median sensitivity. The margin may be divided by the median sensitivity, and optionally including a safety factor. The dose change may be expressed by Equation (6) as follows:

$$Dose change = α \times \frac{margin}{median sensitivity} \qquad ( 6 )$$

The safety factor can be set based on a difference between predicted overnight median of the applied doses and the actual overnight median after the dose change, as set forth in Equation (7) as follows:

$$( 7 )$$

$$Δ OvernightMedian = predictedOvernightMedian - actualOvernightMedian$$

The predictedOvernightMedian can be calculated by following the fit line in FIG. 25 and finding the corresponding y-axis value (overnight median) for the applied dose amount (basal dose amount) shown on the x-axis.

A safety factor, a, may be determined based on a training set for a population. Different safety factor values result in different distributions of the change in overnight median. Generally, the larger the safety factor, the larger the distribution of the change in overnight median.

In some embodiments, the safety factor may be predetermined by a training set for a population. The association between the distribution property of the change in overnight median values given the chosen safety factor is stored. The distribution property may be for example, one or more parameters of Gaussian distribution, mean of the distribution, standard deviation of the distribution, offset, skewness, or kurtosis, among others. Glucose data and insulin data are collected for a user, and when sufficient glucose-insulin data is available for the user, a comparison is made between the distribution property of the change in overnight median values from the user's data against the training set derived quantities. If the scatter of the change in overnight median values from the user's data is larger given the chosen safety factor, the safety factor is gradually decreased. Various numerical approaches can be used to decrease the safety factor, such as a steepest descent method, a Newton-Raphson method, or MIT adaptation rule, among others. For example, if the lower 5% confidence interval of the overnight median as calculated using the distribution property is above a pre-determined lower threshold, the difference between the lower 5% confidence interval and a pre-determined lower threshold can be used as the input variable to the numerical approaches to reduce the safety factor. If the upper 95% confidence interval of the overnight median is below a pre-determined upper threshold, the difference between the upper 95% confidence interval of the overnight median and the pre-determined upper threshold can be used as the input variable to the numerical approaches to increase the safety factor. It is understood that other percentiles can be chosen.

In some embodiments, the safety factor may be determined using a machine learning model. Machine learning models can include, by way of example and not limitation, models trained using or encompassing decision tree analysis, gradient boosting, adaptive boosting, artificial neural networks or variants thereof, linear discriminant analysis, nearest neighbor analysis, support vector machines, supervised or unsupervised classification, and others. The model may receive the glucose data and insulin data of the user, and one or more distribution properties as described above. The model may further include the training set for the population. The model may output a safety factor.

In some embodiments, estimation of the optimal dose can be used to set the initial basal dose when a user is first started on basal insulin therapy. The initial median sensitivity may be set to a default value. The default value may be a large, but realistic value to produce a relatively small optimal dose estimate. For example, the median sensitivity may be set to 2 mg/dl/U, 1.5 mg/dl/U, or 1 mg/dl/U, among other values. The default value may be based on population data for median sensitivity. Starting with a smaller dose helps to minimize the risk of hypoglycemia as the user starts basal insulin therapy. Alternatively, the median sensitivity may be set to an average value, and the safety factor may be set such that the recommended initial basal dose is relatively small.

A further safety factor can be introduced by considering uncertainty arising from noise in glucose data when selecting a median sensitivity. The glucose data used to calculate glucose median and median sensitivity may have noise and other abnormalities. A Gaussian distribution for the glucose median is characterized by a mean and a standard deviation. A sensitivity value can be calculated based on median distributions at different dosages using a least squares method. A sensitivity distribution may be generated by sampling from each median distribution and calculating a sensitivity. The sensitivity distribution provides a quantitative estimate of the risk of hypoglycemia. For example, if a 90th percentile value of median sensitivity is used, there is a 10% risk of overdose. The acceptable risk of overdose is a clinical decision that can be made by the HCP.

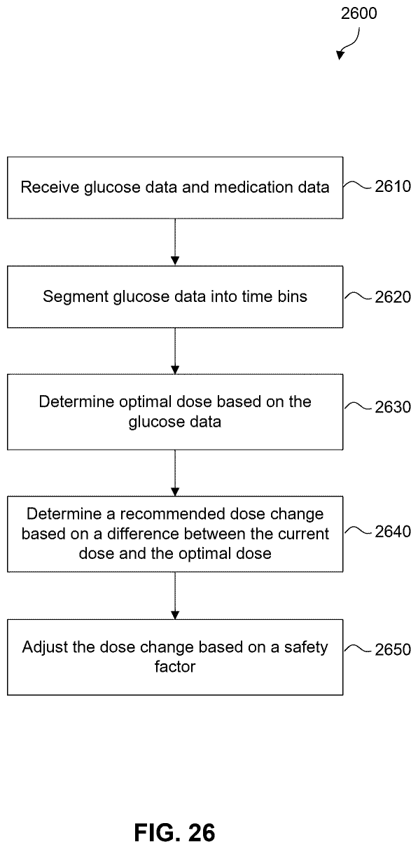

An exemplary method of titrating a basal dose of insulin 2600 is shown in FIG. 26. The dose guidance system may include receiving glucose data and medication data over a predetermined period of time 2610. The glucose data is segmented into one or more time bins 2620. The time bins may include a plurality of TOD periods, such as breakfast, lunch, dinner, and overnight periods. The system may determine an optimal dose based on the glucose data 2630. The optimal dose may be for achieving a goal glucose median based on a current dose, current median, goal median, and median sensitivity as described herein. Alternately, the optimal dose may be based on the margin and median sensitivity. A recommended dose change is determined based on a difference between the current dose and the optimal dose 2640. The dose change may optionally be adjusted based on a safety factor 2650. This may help to reduce incidences of hypoglycemia that may result from a large dose change.

The estimated optimal dose may be used as the maximum dose for titration of the medication dose. If the maximum dose is reached by the dose guidance algorithm, the optimal dose may be reevaluated to determine if an adjustment in the maximum dose is needed. A notification to adjust the maximum dose may be communicated to a computing device of the HCP and displayed in a dose guidance status, as disclosed for example in FIG. 18A-18B. The estimated optimal dose may be communicated to a computing device of an HCP. The HCP may determine if the maximum dose should be increased and can set a new maximum dose. The HCP recommended maximum dose may be communicated to a dose guidance system of a user, such as to a mobile application on a display device of the user.

Titration of a medication dose may result in one or more increases to the initial medication dose to achieve glucose goals. However, the dose may be increased to a medication dose that the dose guidance systems deems to be too high, such as if the medication dose resulted in hypoglycemia or excessive hypoglycemia. As a result, the dose guidance algorithm may then determine that a decrease in the medication dose is needed. The dose guidance algorithm may decrease the medication dose by a fixed amount, or by a fixed percentage of the current medication dose.

The medication dose amount that resulted in the determination to decrease the medication dose may be set as an upper limit of the range for the next medication dose. The most recent prior medication dose at which a dose increase was recommended may be set as the lower limit of the range of the next medication dose. Thus, the next medication dose may be selected as an amount that is between the lower limit and the upper limit dose amounts. The next medication dose may be selected that is an average of the upper limit and the lower limit. Alternatively, a difference between the upper limit and the lower limit may be computed, and the lower limit dose may be increased by a percentage of the difference between the upper limit and the lower limit, as shown by Equation (8) as follows:

$$Next medication dose = {dose}_{LL} + P \times ( {dose}_{UL} - {dose}_{LL} ) \qquad ( 8 )$$

wherein dose_{LL} is the dose lower limit, P is a percentage, and dose_{UL} is the dose upper limit. The percentage may be between 10% and 50%, 20% and 40%, or is 30%.

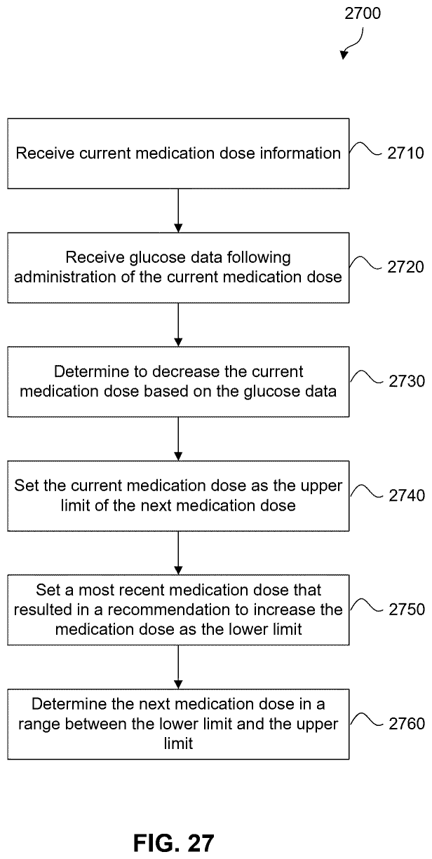

An exemplary method for titrating a medication dose is shown in FIG. 27. The dose guidance system may receive current medication dose information 2710. The system may receive glucose data following administration of the current medication dose 2720. The dose guidance system may determine based on the glucose data to decrease the current medication dose 2730. The dose guidance system sets the current medication dose as the upper limit for the next medication dose amount 2740. A most recent medication dose that resulted in a recommendation by the dose guidance system to increase the medication dose may be set as a lower limit for the next medication dose 2750. The next medication dose may be determined to be a dose in a range of the lower limit and the upper limit 2760. As discussed above, the next dose may be an average of the lower limit dose and the upper limit dose. Alternatively, the difference between the upper limit and lower limit may be computed, and the next medication dose may be the lower limit dose amount plus a predetermined percentage of the difference between the upper and low limit doses.

Medication doses are titrated over time, to increase or decrease the amount of the medication dose, in order to improve glycemic control which may be assessed with reference to one or more glucose metrics. Various methods for titrating a medication dose, such as an insulin dose, such as rapid-acting insulin or long-acting insulin, are described herein. When titrating medication doses, it can be difficult to determine when the medication dose has been fully or optimally titrated. As a result, the medication may be over-titrated, or the therapy may otherwise languish in a repetitive cycle of titration with limited improvement to the patient's condition. Determining when the medication dose is fully or optimally titrated may allow the HCP to initiate a new medication or therapy or to recommend adjustments to self-care behaviors.

Some embodiments described herein relate to systems and methods for determining when a medication dose has been fully or optimally titrated. The system may receive and analyze analyte data, such as glucose data, over a preceding period of time, such as over the previous day, medication data over the preceding period of time, and recommend an adjusted medication dose to achieve one or more target analyte metrics. For example, data collected over a period of days shows that the user administers a daily basal insulin dose and the glucose levels remain elevated above a target glucose level after the basal insulin dose is administered, the system may determine to increase the basal insulin dose amount in order to further reduce the glucose levels toward the target glucose level.

The system may determine that a medication dose, such as an insulin dose, has been fully or optimally titrated based on one or more full titration criteria. The full titration criteria may be based on glucose data, insulin data, or other data and combinations thereof. The full titration criteria may include analysis of the medication dose amounts over time. The full titration criteria may be based on analysis of TOD periods and a corresponding glucose pattern assessment as described herein. The full titration criteria may be based on analyte data or analyte metrics, such as glucose data and glucose metrics. The glucose data or metrics may be compared to a target glucose metric, or may be compared to the corresponding metrics for preceding periods of time. The full titration criteria may include determining that the glucose metric has not changed over a predetermined period of time, or that the change is less than a threshold amount of change, e.g., less than a predetermined percentage. The full titration criteria may include or be based on an amount of time in which no change in medication dose is recommended, an amount of time in which glucose patterns for TOD periods have not changed, change in time in range, or minimum percentile glucose level.

The full titration criteria may include determining that the medication dose has not changed within a predetermined period of time. The minimum predetermined period of time may be 3 days, 5 days, 7 days, 10 days, or 14 days, among other periods of time. The full titration criteria may include determining that the titration decision is to maintain the current medication dose for at least a predetermined period of time, or that the decision to maintain the current medication dose has repeated for a predetermined number of titrations. The full titration criteria may include a mean dose change over a predetermined period of time below a predetermined percentage (e.g., less than 5% change in mean dose over a period of time). Thus, if the dose guidance algorithm analyzes the glucose data and does not determine that a change in the medication dose is needed over a period of time, or over multiple rounds of titration, the medication dose may be deemed to be optimized.

The system may determine that the medication dose is fully titrated when a glucose pattern assessed for one or more TOD periods has not changed over a predetermined period of time, or has not changed for a predetermined number of rounds of titration.

Full titration may be determined when an increase in a glucose time in range (TIR) for a medication dose relative to the TIR for the previously recommended medication dose is less than a target percentage (e.g., 5%). For example, if a first medication dose resulted in a TIR of 60%, and the dose guidance system recommends a second medication dose and the second medication dose resulted in a TIR of 61%, the medication dose may be deemed to be fully titrated. The full titration criteria may include determining that the glucose median is within a predetermined amount of the median goal, such as within 10% or less, 8% or less, or 6% or less of the median goal. The full titration criteria may include determining that the glucose median has changed less than a target percentage.

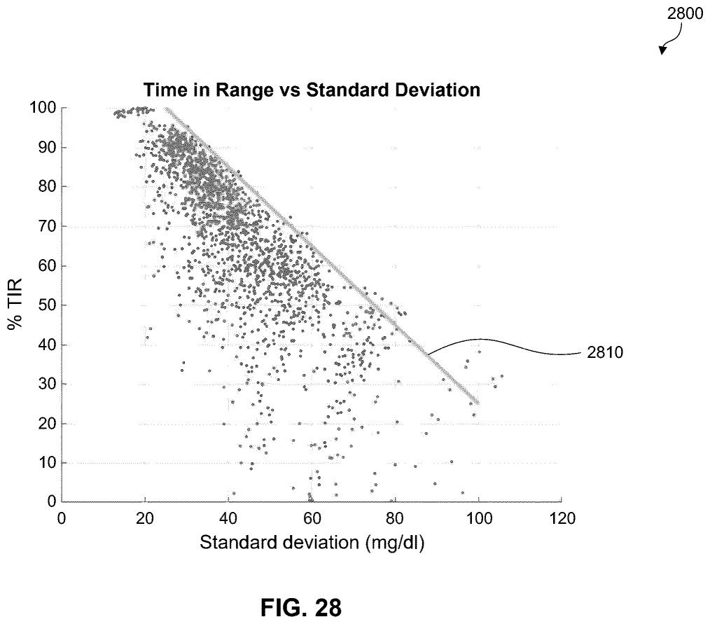

The full titration criteria may include determining that the time in range is within a predetermined amount of a maximum time in range. The maximum time in range may be determined based on glucose variability. The glucose variability may be a standard deviation. Simulated data demonstrated a sharp cut-off for time in range for a given standard deviation as shown for example in the plot 2800 of FIG. 28. A linear fit line 2810 represents the maximum time in range for a given standard deviation.

The full titration criteria may include a determination that a minimum glucose percentile is less than a threshold glucose level. The minimum glucose percentile may be a minimum 4^{th} percentile of glucose data. The minimum glucose percentile may be calculated by segmenting glucose data in a 24-hour period into time bins of a fixed amount of time (e.g., 2 hours). The full titration criteria may be based on the lowest value of the glucose percentile from the 2 hour time bins being less than a predetermined low glucose level (e.g., 70 mg/dl). For basal dose titration, the minimum 4^{th} percentile can be computed by comparing all of the time bins. For meal-time doses, the minimum 4^{th} percentile can be calculated by comparing all of the time bins corresponding to each mealtime dose, e.g., all time bins in the respective TOD period.

If the dose guidance system determines that the medication dose has been fully titrated, such as based on one or more of the full titration criteria being satisfied, the dose guidance system may perform one or more actions. The action may include outputting a notification that the medication dose is fully titrated. The action may include outputting a recommendation for initiating a new medication regimen or a recommendation to discuss initiating a new medication regimen with the patient's HCP. For example, if the patient is taking a mealtime insulin dose (prandial insulin dose), the system can recommend adding a mealtime insulin dose for an additional meal, escalating existing medications, or adding new medications. The system may alternatively or additionally recommend one or more self-care behaviors, such as recommendations relating to diet or exercise, among others. The action may include stopping or preventing further titration of the insulin dose, such that no further recommendations to increase, decrease, or maintain the insulin dose are output to the patient. The action may include adjusting a period of time over which the insulin dose is titrated. For example, subsequent determinations to increase, decrease or maintain the insulin dose may be based on glucose data collected over a second, longer period of time than the first period of time.

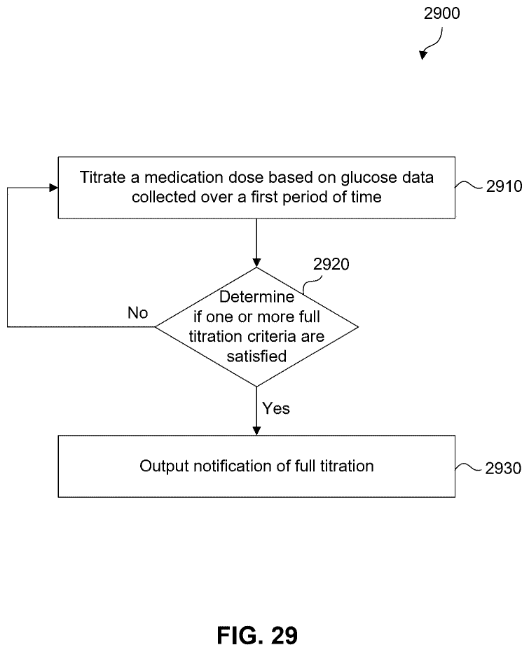

An exemplary method of determining full titration of a medication dose is shown for example at FIG. 29. The method 2900 includes titrating a medication dose based on glucose data collected over a first period of time 2910. Titrating medication dose may include receiving glucose data following administration of the medication dose over a period of time, and determining to increase, decrease, or maintain the medication dose amount based on the glucose data received during the period of time. The system determines if one or more full titration criteria 2920 are satisfied. If the medication dose is not fully titrated, i.e., the one or more full titration criteria are not satisfied, the method 2900 repeats titrating the medication dose 2910 (e.g., by collecting glucose data following a second medication dose over a second period of time and determining and outputting a recommendation based on the glucose data whether to increase, decrease or maintain the medication dose). If one or more full titration criteria are satisfied, the system may perform an action, such as outputting a notification to the user 2930. The notification may be output on a display device of the user or on a computing device of an HCP or both.

When a patient is on a medication regimen that includes only basal insulin, one or more additional full titration criteria may be applied. The additional full titration criteria for basal insulin titration may include that the current dose has reached or exceeded a maximum dose. The maximum dose for a patient may be based on a body weight of the patient. The full titration criteria may include a ratio of the basal dose to the user's body weight exceeding a basal dose to body weight threshold. The threshold may be for example, 0.6 U/kg body weight.

The full titration criteria may include a decrease in overnight glucose levels beyond a threshold. The decrease in overnight glucose levels may be determined based on a bedtime to morning (“BEAM”) value that is greater than a threshold glucose level decrease, e.g., 50 mg/dl. The BEAM value may be determined as a difference between a glucose level at midnight and the lowest overnight value.

The full titration criteria may include a fasting glucose level is acceptable, but other glycemic goals are not met. The glycemic goals may include a goal time in range at or above a threshold, e.g., 70%, 75%, or 80%. The glycemic goals may include a glucose management indicator (GMI) at a target level, e.g., 7.5%.

If the patient is only taking basal insulin, and the basal insulin dose is determined to be fully titrated, such as based on the full titration criteria described herein, the system may recommend initiating a new medication regimen, such as initiating one or more prandial insulin doses, or adding a new medication.

The initial medication dose titration and determination of full titration may be determined based on analysis of glucose data over a first period of time, such as 7 days. For example, a glucose pattern for each TOD period may be based on 7 days of glucose data. Determining titration over a larger number of days may help to reduce uncertainty and improve accuracy of pattern determination. Once the medication dose is determined to be fully titrated, the dose guidance system may no longer recommend changes to the medication dose. The dose guidance system may continue to analyze glucose data and titrate the medication dose, but the glucose pattern analysis used to titrate the medication dose is based on a larger amount of data, such as glucose data over a second period of time, such as 14 days. Thus, titrations are recommended if there is strong evidence of a pattern change (based on the larger number of days of glucose data).

The titration of the medication dose may be stopped if a high risk of hypoglycemia is detected. The risk of hypoglycemia may be based on a time below range, or time below a predetermined minimum glucose level exceeding a threshold. The risk of hypoglycemia may be based on detection of a Low pattern by the glucose pattern analysis as described herein. The risk of hypoglycemia may be determined when a distance of a glucose median to a hypoglycemia risk line on a plot of glucose median over glucose variability exceeds a threshold.

The titration of the medication dose may be stopped when there is a change in the user's therapy or medication regimen. The change may be entered by the patient or health care provider, such as by inputting a new medication and dosage into the system, or recording administration of a new medication dose. A new therapy, such as a new medication, may be retrieved from an electronic medical record (EMR) in communication with the medication system.

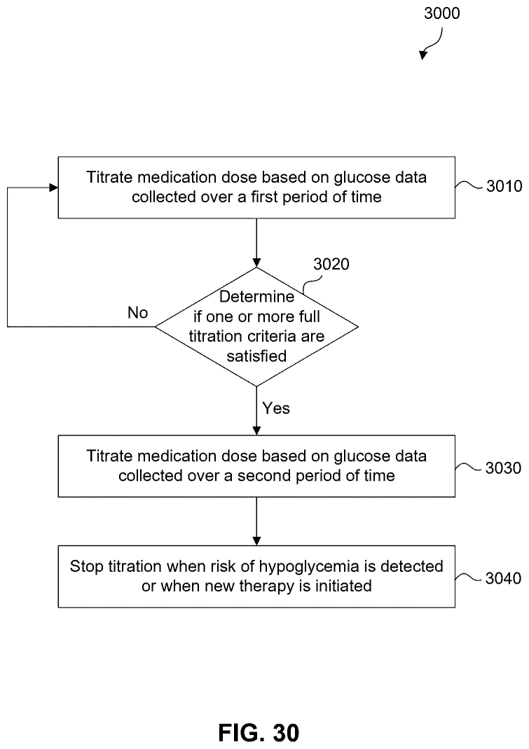

An exemplary method of titrating a medication dose 3000 is shown in FIG. 30. A medication dose is titrated based on glucose data from a first period of time 3010. For example, the medication dose may be determined based on analyte data collected over the preceding 7 days. The system may determine if one or more full titration criteria are satisfied 3020. Full titration of the medication dose may be determined as described herein, such as with respect to method 2900. Once the medication dose is fully titrated, the system may continue to titrate the medication dose based on analyte data for a second period of time 3030. The second period of time is longer than the first period of time, and may be for example 14 days. Further titration of the mediation dose may be stopped when a risk of hypoglycemia is detected or when a new therapy is initiated 3040.

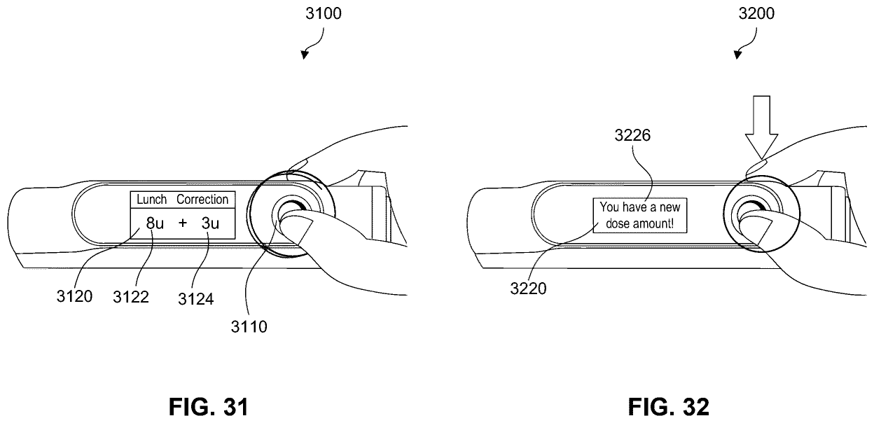

In some embodiments described herein, dose guidance system may include a dose monitoring device. The dose monitoring device may be configured as a smart pen cap, as shown for example in FIG. 31. Smart pen cap 3100 may be configured to be positioned on a medication injection pen and is configured to automatically capture medication information, such as a type of medication, timing of medication administration, amount of medication administered, among other data. Smart pen cap 3100 may communicate, such as by wireless communication, with one or more other components of dose guidance system, such as a display device of a user, a continuous glucose monitoring device, and/or a network. Smart pen cap 3100 may transmit and receive medication information and/or glucose information. The smart pen cap 3100 may include an input 3110, such as a button, that can be operated to turn the pen cap on or off, and to change information shown on a display 3120 of pen cap 3100. Display 3120 may show a current glucose level as determined by a glucose monitoring device in communication with smart pen cap 3100, a glucose trend arrow. Smart pen cap 3100 may show medication information, such as a time since the last dose was administered. Smart pen cap 3100 may display a recommended medication dose. The recommended medication dose may include a meal dose amount 3122. The meal dose amount may be based on the dose amounts for each meal determined by dose guidance system. Smart pen cap 3100 may further display a recommended correction dose amount 3124 based on the user's current glucose level. Display 3120 may show the meal dose and the correction dose amounts simultaneously, or may display the recommended dose amount as a sum of the two dose amounts.

As shown in FIG. 32, a dose monitoring device, such as a smart pen cap, is shown in FIG. 32. Smart pen cap 3200 may have similar features as described above with respect to smart pen cap 3100. Smart pen cap 3200 may be configured for use with an injection pen for delivering long-acting doses of insulin to a user. Display 3220 of smart pen cap 3200 may display a recommended dose of long-acting insulin. Further, when the long-acting insulin dose is adjusted, such as due to titration of the dose amount by the dose guidance system as described herein, display 3220 may present a notification 3226 alerting the user that the dose amount has been updated. Display 3220 may further present the new dose amount to the user. This may help the user to stay informed as to changes to the user's therapy. The user may also select to modify the recommended dose amount. Presenting the dose amount to the user gives the user the opportunity to adjust the new dose amount.

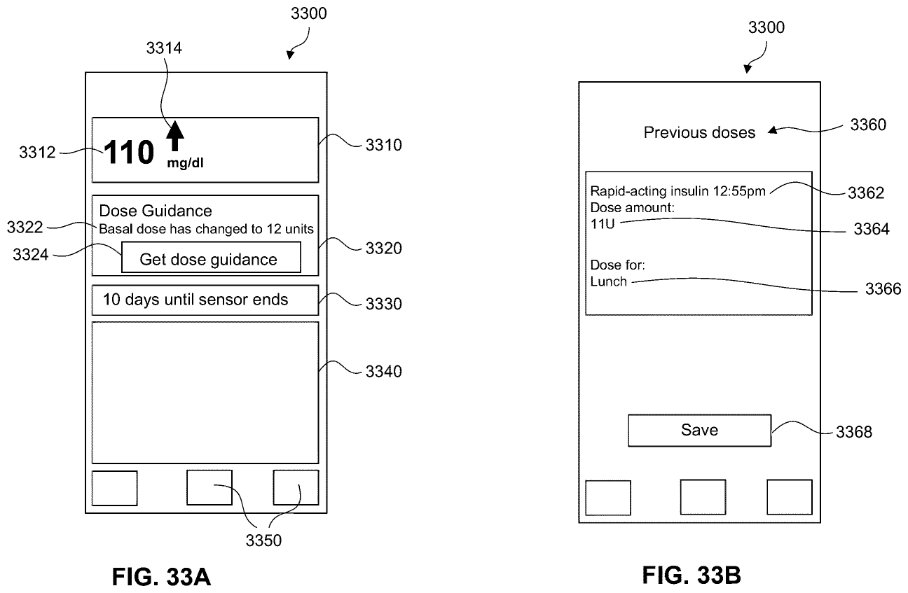

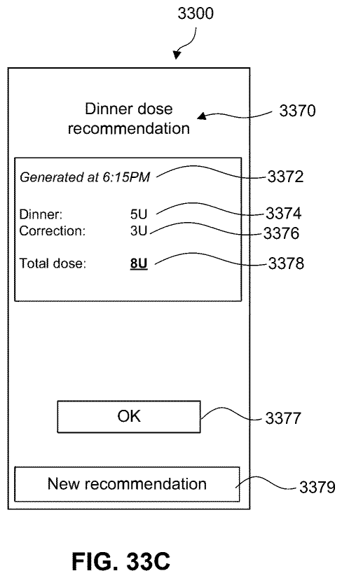

An exemplary user interface for a glucose monitoring application incorporating dose guidance is shown for example in FIGS. 33A-33C. User interface 3300 may include a home screen for a glucose monitoring application. Glucose monitoring application may be in communication with a glucose monitoring device as described herein. Glucose monitoring application may be configured to receive, process and display glucose data, including various glucose metrics and information about the glucose monitoring device. User interface 3300 may display medication information in addition to glucose data. User interface 3300 may include a plurality of different panels for displaying information. However, other display formats may be used.

As shown in FIG. 33A, user interface 3300 may show current glucose information 3310. Current glucose information 3310 may include a real-time glucose level 3312, such as a most recently received glucose level. Current glucose information 3310 may include a glucose trend indicator, such as an arrow 3314. User interface 3300 may further display glucose monitoring device information 3330. Glucose monitoring device information 3330 may include information about sensor expiration, such as a remaining life of the glucose sensor. A countdown of a remaining sensor life may be displayed. User interface 3300 may display various additional content 3340 including educational resources, tips, instructions and other guidance for the user. Additional content 3340 may include further glucose information, such as a glucose trend graph, among others. User interface 3300 may include one or more menu icons 3350 for easily navigating to other screens of the user interface or for performing additional actions, such as logging one or more events.

User interface 3300 may include a dose guidance section 3320. Dose guidance section 3320 may include information about the user's medication regimen 3322, including any updates to doses as recommended by the dose guidance system or received from the HCP. Dose guidance section 3320 may include a selectable icon 3324 to receive dose guidance. The user may select the icon 3324 to receive guidance for administration of a dose of medication, such as for a meal dose or a correction dose, among others.

Selection of the dose guidance icon 3324 may prompt the user to confirm a most recent medication dose, such as the amount of the dose and the type of the dose (e.g., lunch dose). A new window may be presented to confirm the most recent dose, or a pop-up window may be generated, among other presentations. FIG. 33B shows an exemplary user interface 3300 showing one or more previously administered doses 3360. The previous doses may be auto-populated by the software, or may require manual user entry of the previous dose information. The previous dose may include the dose information, such as the medication type (e.g., rapid-acting insulin). Dose information may include the time of the dose 3362, the dose amount 3364, and the purpose of the dose 3366, e.g., meal dose (or more specifically a breakfast, lunch or dinner dose). The dose amount and purpose of the dose may be editable, such that the user can adjust as necessary. A selectable icon or button 3368 may be displayed to save the dose information or to confirm the dose information 3368.

Upon receiving the user confirmation of a most recent medication dose, user interface 3300 may present a dose recommendation as shown, for example, in FIG. 33C. The dose recommendation 3370 may include a time of the recommendation 3372. If the user does not immediately take a dose after requesting the dose recommendation, the time 3372 may help the user to decide if the recommendation is sufficiently recent or if an updated recommendation should be generated. The dose recommendation 3370 may include a meal dose amount 3374, a correction dose amount 3376, or both. Where the dose recommendation includes a meal dose amount 3374 and a correction dose amount 3376, a total dose amount 3378 may be displayed in addition to or in place of the meal dose amount 3376 and correction dose amount 3378. Interface 3300 may include a selectable icon or button 3377 to confirm the dose recommendation. Selecting the icon or button 3377 may return to the home screen. Interface 3300 may include a selectable button or icon 3379 to receive a new dose recommendation. This may be used, for example, where time has passed since an initial dose recommendation as generated.

Exemplary embodiments are set out in the following numbered clauses:
Clause 1. A method for titrating a medication dose, the method comprising: receiving, by a processor, glucose data collected by a glucose monitoring device worn by a user over each of a plurality of time of day periods, wherein the glucose monitoring device is in communication with the processor;
determining, by the processor, a time of administration of each of a plurality of medication doses taken by the user;
detecting, by the processor, a fasting period based on one or more of the glucose data and the times of administration of the plurality of medication doses;
determining, by the processor, a change in a schedule of a user based on a detected change in the fasting period;
adjusting, by the processor, the plurality of time of day periods based on the determined change in the schedule of the user; and
titrating the medication doses for each time of day period of the adjusted plurality of time of day periods.
Clause 2. The method of clause 1, wherein the fasting period corresponds to a longest interval between consecutive medication doses of the plurality of medication doses.
Clause 3. The method of any one of clauses 1 and 2, wherein detecting the fasting period comprises determining a variability in the glucose data for a plurality of moving windows of time, and wherein the fasting period comprises a moving window of time of the plurality of moving windows of time having the lowest glucose variability.
Clause 4. The method of any one of clauses 1 to 3, wherein detecting the change in the fasting period comprises detecting the start time of the fasting period has changed from a first time to a second time, and the start time of the fasting period is the second time for a predetermined minimum period of time.
Clause 5. The method of any one of clauses 1, 2 and 4, wherein detecting the fasting period comprises autocorrelation or cross-correlation of the glucose data.
Clause 6. The method of any one of clauses 1 to 5, wherein the glucose monitoring device comprises an in vivo glucose sensor comprising a first portion positioned below the skin and in contact with a bodily fluid and a second portion positioned above the skin.
Clause 7. The method of any one of clauses 1 to 6, wherein the medication doses comprise insulin doses, wherein the insulin doses comprise fast-acting insulin, slow-acting insulin, or pre-mixed insulin.
Clause 8. The method of any one of clauses 1 to 7, wherein the plurality of time of day periods comprise a plurality of mealtime periods.
Clause 9. The method of clause 8, wherein the plurality of time of day periods comprises a breakfast period, a lunch period, a dinner period, and an overnight period.
Clause 10. A dose guidance system comprising a processor and a memory, the memory comprising an algorithm which, which executed by the processor, causes the processor to perform the method of any one of clauses 1 to 9.
Clause 11. A non-transitory computer-readable medium comprising an algorithm which, when executed by a computer, causes the computer to perform the method of any one of clauses 1 to 9.
Clause 12. A method for titrating a medication dose, the method comprising: receiving, by one or more processors, glucose data from a continuous glucose monitoring device over a predetermined period of time;
receiving, by the one or more processors, medication data comprising a time and an amount of one or more medication doses;
segmenting, by the one or more processors, the glucose data into a plurality of glucose data segments, wherein the glucose data segments comprise glucose data segments associated with medication doses;
performing, by the one or more processors, a glucose pattern analysis for one or more glucose data segments of the plurality of glucose data segments;
performing, by the one or more processors, an event counting analysis on one or more of the plurality of glucose data segments;
determining, by the one or more processors, to increase, decrease or maintain the medication dose based on the glucose pattern analysis and the event counting analysis for the associated glucose data segment; and
outputting, on a display device in communication with the one or more processors, a recommendation to increase, decrease or maintain the medication dose.
Clause 13. The method of clause 12, wherein the glucose data segments comprise a meal segment, wherein the meal segment comprises a start time corresponding to a time of administration of a medication dose, and an end time that is one of a start time of a subsequent medication dose or a fixed amount of time following the start time.
Clause 14. The method of clause 13, wherein the glucose data segments include an idle segment, wherein the idle segment comprises a start time at an end of the fixed amount of time following the start time of the meal segment and that has an end time at a start time of a subsequent medication dose.
Clause 15. The method of clause 14, wherein the event counting analysis comprises counting a number of low glucose events or low glucose alarms in each of the meal segments and the idle segments.
Clause 16. The method of clause 15, wherein titrating the medication dose comprises decreasing a basal dose if the number of low glucose events or low glucose alarms in one or more of the idle segments exceeds a threshold number of low glucose events or low glucose alarms.
Clause 17. The method of clause 15, wherein titrating the medication dose comprises decreasing a medication dose associated with a meal if the number of low glucose events or low glucose alarms in the associated glucose data segment exceeds a threshold number of low glucose events or low glucose alarms.
Clause 18. The method of any one of clauses 14 to 17, further comprising determining an overnight period from the idle segment when a pattern of glucose data segments comprises an idle segment followed by a breakfast segment, and wherein the overnight period has an end time at the end of the idle segment and a start time that is a fixed number of hours before the end time of the overnight period.
Clause 19. The method of clause 18, wherein the glucose pattern analysis comprises determining a glucose pattern for each of the meal segments and the overnight period.
Clause 20. The method of any one of clauses 12 to 19, further comprising determining validity of the glucose data segments prior to the event counting analysis or the glucose pattern analysis.
Clause 21. The method of clause 20, wherein determining the validity of the glucose data segments comprises deeming glucose data segments invalid that have a start time that is more than a predetermined amount of time before a most recent glucose data segment.
Clause 22. The method of any one of clauses 12 to 21, wherein the event counting analysis comprises determining for each meal segment, a number of correction doses following the meal segment.
Clause 23. The method of clause 22, further comprising adjusting a glucose pattern for the meal segment if the number of correction doses following the meal segment exceeds a threshold number of correction doses.
Clause 24. The method of any one of clauses 12 to 23, further comprising determining a medication dose of the one or more medication doses is a meal dose when the dose amount corresponds to a recommended dose amount.
Clause 25. The method of any one of clauses 12 to 24, further comprising determining a medication dose of the one or more medication doses is a meal dose when a time of administration of the medication dose is within a predetermined period of time from a recommendation for the meal dose.
Clause 26. The method of any one of clauses 12 to 25, further comprising classifying a medication dose as a breakfast, lunch, or dinner dose, respectively, based on the time of administration of the dose being within a predetermined time range for one of the breakfast, lunch, or dinner dose.
Clause 27. A dose guidance system comprising a processor and a memory, the memory comprising an algorithm which, which executed by the processor, causes the processor to perform the method of any one of clauses 12 to 26.
Clause 28. A non-transitory computer-readable medium comprising an algorithm which, when executed by a computer, causes the computer to perform the method of any one of clauses 12 to 26.
Clause 29. A method of titrating a medication dose, the method comprising: receiving, by a processor, glucose data collected by a glucose monitoring device comprising a first portion positioned under a skin surface of a user and in contact with a bodily fluid;
determining, by the processor, a glucose median for a time of day period based on the glucose data collected by the glucose monitoring device;
determining, by the processor, a hypoglycemic risk based on comparison of a glucose median of a user to a hypoglycemia risk threshold for the time of day period; and
outputting, by the processor, a recommendation to increase the medication dose by an amount based on the hypoglycemic risk.
Clause 30. The method of clause 29, wherein the recommended increase in the medication dose is proportional to the hypoglycemic risk.
Clause 31. The method of any one of clauses 29 to 30, further comprising:
categorizing the determined hypoglycemic risk into a first bin when the hypoglycemic risk is below a hypoglycemic risk threshold or a second bin when the hypoglycemic risk is above the hypoglycemic risk threshold; and
wherein the amount is a first amount when the hypoglycemic risk is categorized into the first bin, and the amount is a second amount that is greater than the first amount when the hypoglycemic risk is categorized into the second bin.
Clause 32. The method of any one of clauses 29 to 31, wherein the hypoglycemic risk comprises a vertical distance of a glucose median to a hypoglycemia risk curve on a plot of glucose median over glucose variability, wherein the hypoglycemia risk curve comprises a set of points having the same value for a hypoglycemia risk metric.
Clause 33. The method of any one of clauses 29 to 32, wherein the amount of increase comprises a percentage of the medication dose.
Clause 34. The method of any one of clauses 29 to 32, wherein the amount of increase comprises a number of units by which to increase the medication dose.
Clause 35. The method of any one of clauses 29 to 34, further comprising determining a hypoglycemic risk for a first meal period and for a second meal period following the first meal period, and recommending an increase in the medication dose for each of the first and the second meal periods when the hypoglycemic risk of the second meal period is greater than the hypoglycemic risk of the first meal period.
Clause 36. A dose guidance system comprising a processor and a memory, the memory comprising an algorithm which, which executed by the processor, causes the processor to perform the method of any one of clauses 29 to 35.
Clause 37. A non-transitory computer-readable medium comprising an algorithm which, when executed by a computer, causes the computer to perform the method of any one of clauses 29 to 35.
Clause 38. A method for titrating an insulin dose, the method comprising:
receiving, by a processor, medication information comprising time of administration of one or more medication doses;
receiving low glucose event data from a continuous glucose monitor in communication with the processor;
associating, by the processor, a low glucose event with a meal dose if the low glucose event occurs within a first period of time following administration of the meal dose;
associating, by the processor, a low glucose event with a basal dose when the low glucose event does not occur within the first period of time, and the low glucose event occurs within a second period of time following the administration of the basal dose; and
recommending a decrease in the associated meal dose or basal dose if the number of low glucose events exceeds a threshold number of low glucose events for the meal dose or the basal dose.
Clause 39. The method of clause 38, wherein the low glucose event data comprises low glucose alarms output by the continuous glucose monitor.
Clause 40. The method of any one of clauses 38 and 39, wherein the low glucose event data comprises instances in which a minimum number of glucose levels is below a low glucose threshold in a period of time.
Clause 41. The method of any one of clauses 38 to 40, wherein the second period of time is 24 hours.
Clause 42. The method of any one of clauses 38 to 41, wherein the threshold number of low glucose events differs for the meal dose and for the basal dose.
Clause 43. A dose guidance system comprising a processor and a memory, the memory comprising an algorithm which, which executed by the processor, causes the processor to perform the method of any one of clauses 38 to 41.
Clause 44. A non-transitory computer-readable medium comprising an algorithm which, when executed by a computer, causes the computer to perform the method of any one of clauses 38 to 41.
Clause 45. A method of titrating an insulin dose, the method comprising:
receiving, by a processor, glucose data from a glucose monitoring device worn by a user;
receiving, by the processor, medication data relating to insulin doses taken by the user;
identifying, by the processor, a glucose pattern for each of a plurality of time of day periods based on the glucose data;
determining, by the processor, to decrease a medication dose for a time of day period of the plurality of time of day periods based on the identified glucose pattern, wherein the determination to decrease the medication dose is based on a first number of days of glucose data in each of the plurality of time of day periods;
determining, by the processor, to increase a medication dose for a time of day period of the plurality of time of day periods based on the identified glucose pattern, wherein the determination to increase the medication dose is based on a second number of days of glucose data in each of the plurality of time of day periods, wherein the second number of days is greater than the first number of days; and
outputting, on a display of a display device in communication with the processor, a recommendation to decrease or increase the medication dose.
Clause 46. The method of clause 45, wherein the plurality of time of day periods correspond to mealtimes.
Clause 47. The method of clause 45 or clause 46, wherein the medication doses comprise one or more of rapid-acting insulin doses or long-acting insulin doses.
Clause 48. A dose guidance system comprising a processor and a memory, the memory comprising an algorithm which, which executed by the processor, causes the processor to perform the method of any one of clauses 45 to 47.
Clause 49. A non-transitory computer-readable medium comprising an algorithm which, when executed by a computer, causes the computer to perform the method of any one of clauses 45 to 47.
Clause 50. A method of titrating an insulin dose, the method comprising:
receiving, by a processor, glucose data from a glucose monitoring device worn by a user;
receiving, by the processor, medication data relating to medication doses administered by the user;
identifying, by the processor, a glucose pattern for each of a plurality of time of day periods based on the glucose data;
determining, by the processor, to increase a rapid-acting insulin dose for a first time of day period of the plurality of time of day periods based on the identified glucose pattern when a minimum number of days of glucose data is available for the first time of day period and for a consecutive time of day period;
determining, by the processor, to increase a long-acting insulin dose based on the identified glucose patterns for the plurality of time of day periods when the minimum number of days of glucose data is available for each of the plurality of time of day periods; and
outputting, on a display of a display device in communication with the processor, a recommendation to increase the rapid-acting insulin dose or the long-acting insulin dose.
Clause 51. The method of clause 50, wherein the plurality of time of day periods correspond to mealtimes.
Clause 52. The method of clause 50 or 51, wherein the minimum number of days of glucose data is in a range of 5 to 10 days.
Clause 53. A dose guidance system comprising a processor and a memory, the memory comprising an algorithm which, which executed by the processor, causes the processor to perform the method of any one of clauses 50 to 52.
Clause 54. A non-transitory computer-readable medium comprising an algorithm which, when executed by a computer, causes the computer to perform the method of any one of clauses 50 to 52.
Clause 55. A method of titrating parameters of a dose guidance system, the method comprising:
receiving, at an input of a display device, a user entered value for a first parameter of the dose guidance system;
selecting, by the dose guidance system, a value for a second parameter based on the value entered for the first parameter;
titrating one or more of the first parameter and second parameter based on the glucose data of the user;
determining if a titration limit for the first parameter or the second parameter is reached; and
outputting a notification when the titration limit has been reached.
Clause 56. The method of clause 55, wherein the first parameter and the second parameter are titrated independently of one another.
Clause 57. The method of clause 55, wherein the second parameter is in a fixed relationship to the first parameter.
Clause 58. The method of any one of clauses 55 to 57, wherein the first parameter is a pre-meal correction factor and the second parameter is a post-meal correction factor.
Clause 59. The method of any one of clauses 55 to 58, further comprising receiving user entry of the titration limit.
Clause 60. The method of any one of clauses 55 to 59, wherein the titration limit comprises a maximum total daily dose.
Clause 61. The method of any one of clauses 55 to 60, wherein the first parameter comprises a first titration limit and the second parameter comprises a second titration limit.
Clause 62. The method of clause 61, wherein the first titration limit is a total daily dose, and the second parameter is a minimum correction factor.
Clause 63. The method of any one of clauses 55 to 62, wherein outputting a notification when the titration limit has been reached comprises outputting an alert on a display device of the user.
Clause 64. The method of any one of clauses 55 to 63, wherein outputting a notification when the titration limit has been reached comprises providing a notification in a dose guidance status section of a user interface.
Clause 65. A dose guidance system comprising a processor and a memory, the memory comprising an algorithm which, which executed by the processor, causes the processor to perform the method of any one of clauses 55 to 64.
Clause 66. A non-transitory computer-readable medium comprising an algorithm which, when executed by a computer, causes the computer to perform the method of any one of clauses 55 to 64.
Clause 67. A method of notifying a user of a late medication dose, the method comprising:
receiving, by a processor, glucose data from a glucose monitoring device in wireless communication with the processor;
detecting, by the processor, a meal based on the glucose data received from the glucose monitoring device;
outputting, by a display device in communication with the processor, a notification when the meal is detected during a predetermined late dose period, wherein the notification alerts the user of the missed medication dose; and
recommending an insulin dose during the predetermined late dose period.
Clause 68. The method of clause 67, wherein the recommended insulin dose is based on the glucose level at a start time of the detected meal.
Clause 69. The method of any one of clauses 67 to 68, further comprising stopping output of the notification when the predetermined late dose period has elapsed.
Clause 70. The method of any one of clauses 67 to 69, further comprising outputting a second notification at the end of the predetermined late dose period, wherein the second notification indicates that the insulin dose is no longer recommended.
Clause 71. The method of any one of clauses 67 to 70, further comprising stopping output of the notification when an insulin dose is administered.
Clause 72. The method of any one of clauses 67 to 71, further comprising receiving user input declining the recommended medication dose, and stopping outputting the notification when the user input declining the recommended medication dose is received.
Clause 73. The method of any one of clauses 67 to 72, wherein the predetermined late dose period is in a range of 1 to 3 hours.
Clause 74. A dose guidance system comprising a processor and a memory, the memory comprising an algorithm which, which executed by the processor, causes the processor to perform the method of any one of clauses 67 to 73.
Clause 75. A non-transitory computer-readable medium comprising an algorithm which, when executed by a computer, causes the computer to perform the method of any one of clauses 67 to 73.
Clause 76. A method of setting an initial value for rapid-acting insulin doses in a dose guidance system, the method comprising:
receiving, by a processor, glucose data collected by a glucose monitoring device during a learning period;
receiving, by the processor, medication data collected during the learning period, wherein the medication data comprises an amount and a time of administration for each of a plurality of rapid-acting doses;
classifying, by the processor, the rapid-acting doses as one of a plurality of mealtime doses;
extracting, by the processor, one or more glucose features from post-prandial glucose data following each of the one or more mealtime doses;
adjusting, by the processor, the mealtime doses to optimize the one or more glucose features; and
initializing, by the processor, each of the mealtime doses in the dose guidance system based on the adjusted mealtime doses.
Clause 77. The method of clause 76, wherein the plurality of mealtime doses comprise a breakfast dose, a lunch dose, and a dinner dose.
Clause 78. The method of clause 76 or 77, wherein classifying the rapid-acting doses into the plurality of mealtime doses comprises a cluster analysis based on the time of administration of each of the rapid-acting doses during the learning period.
Clause 79. The method of any one of clauses 76 to 78, wherein a median time of administration of a cluster of rapid-acting doses is determined as the mealtime.
Clause 80. The method of any one of clauses 76 to 79, wherein the post-prandial glucose data comprises a predetermined period following the mealtime.
Clause 81. The method of any one of clauses 76 to 80, wherein the one or more glucose features include time in range.
Clause 82. The method of any one of clauses 76 to 81, wherein the one or more glucose features include a hypoglycemia metric.
Clause 83. The method of any one of clauses 76 to 82, wherein the post-prandial glucose data comprises glucose data in a predetermined period of time following the mealtime, wherein the predetermined period of time is 2 hours.
Clause 84. A dose guidance system comprising a processor and a memory, the memory comprising an algorithm which, which executed by the processor, causes the processor to perform the method of any one of clauses 76 to 83.
Clause 85. A non-transitory computer-readable medium comprising an algorithm which, when executed by a computer, causes the computer to perform the method of any one of clauses 76 to 83.
Clause 86. A method of setting an initial value for a basal insulin dose in a dose guidance system, the method comprising:
receiving, by a processor, glucose data collected by a glucose monitoring device during a learning period;
receiving, by the processor, medication data collected during the learning period, wherein the medication data comprises a plurality of basal insulin doses;
extracting, by the processor, one or more glucose features from a period of time following administration of each basal insulin dose;
adjusting, by the processor, a basal insulin dose amount to optimize the one or more glucose features; and
initializing, by the processor, the basal insulin dose in the dose guidance system based on the adjusted basal insulin dose amount.
Clause 87. A dose guidance system comprising a processor and a memory, the memory comprising an algorithm which, which executed by the processor, causes the processor to perform the method of clause 86.
Clause 88. A non-transitory computer-readable medium comprising an algorithm which, when executed by a computer, causes the computer to perform the method of clause 86.
Clause 89. A method of titrating a medication dose, the method comprising:
receiving glucose data from a glucose monitoring device worn on a body of a user, wherein the glucose data is received following administration of a medication dose to the user;
determining to increase, decrease, or maintain an amount of the medication dose based on the glucose data;
when determining to decrease the amount of the medication dose, determining an amount of a next medication dose comprises:
setting the medication dose that resulted in a recommendation to decrease the medication dose as an upper limit;
setting a largest medication dose that resulted in a recommendation to increase the medication dose as a lower limit; and
recommending a next medication dose that is in a range of the upper limit and the lower limit.
Clause 90. The method of clause 89, wherein the recommended dose of the next medication dose is an average of the upper limit and the lower limit.
Clause 91. The method of clause 89 or 90, further comprising determining one or more glucose metrics and one or more medication metrics, and determining the medication dose is fully titrated when a glucose metric of the one or more glucose metrics or a medication metric of the one or more medication metrics meets or exceeds a threshold level.
Clause 92. The method of clause 91, wherein the one or more glucose metrics comprises a time in range, and wherein the medication dose is determined to be fully titrated when the time in range is within a predetermined percentage of a maximum time in range.
Clause 93. The method of clause 92, wherein the maximum time in range is determined based on a relationship of time in range and a standard deviation of the glucose levels of the user over a period of time.
Clause 94. The method of any one of clauses 89 to 93, wherein the one or more glucose metrics comprises a glucose median, and wherein the medication dose is determined to be fully titrated when the glucose median is within a predetermined percentage of a target glucose median.
Clause 95. The method of any one of clauses 89 to 94, wherein the one or more medication metrics comprises a ratio of dose per body weight, and wherein the medication dose is determined to be fully titrated when the ratio of dose per body weight meets or exceeds a threshold ratio of dose to body weight.
Clause 96. The method of any one of clauses 89 to 95, wherein the one or more glucose metrics comprises a difference between a glucose level at midnight and a lowest overnight glucose level, and wherein the medication dose is determined to be fully titrated when the difference is greater than a difference threshold.
Clause 97. A dose guidance system comprising a processor and a memory, the memory comprising an algorithm which, which executed by the processor, causes the processor to perform the method of any one of clauses 89 to 96.
Clause 98. A non-transitory computer-readable medium comprising an algorithm which, when executed by a computer, causes the computer to perform the method of any one of clauses 89 to 96.
Clause 99. A method of titrating an insulin dose, the method comprising:
receiving, by a processor, glucose data from a continuous glucose monitoring device worn on a body of a user collected over a predetermined period of time;
receiving, by the processor, medication data relating to insulin doses administered by the user over the predetermined period of time;
determining an optimal dose based on the glucose data; and
determining an amount of a dose change to a current dose based on a difference between the current dose and the optimal dose.
Clause 100. The method of clause 99, wherein the optimal dose is based on a difference between a current glucose median and a goal median.
Clause 101. The method of clause 100, wherein the goal median comprises a difference between the current glucose median and a margin, wherein the margin comprises a difference between a low percentile glucose level and a hypoglycemia threshold.
Clause 102. The method of clause 101, wherein the low percentile glucose level comprises a 4^{th} percentile glucose value.
Clause 103. The method of any one of clauses 99 to 102, wherein the optimal dose is further based on a median sensitivity.
Clause 104. The method of clause 103, wherein the median sensitivity is based on population data.
Clause 105. The method of clause 103 or 104, wherein the median sensitivity is determined based a linear fit of data points indicative of the glucose median of the user following administration of a basal insulin dose.
Clause 106. The method of any one of clauses 99 to 105, further comprising adjusting the amount of the dose change based on a safety factor.
Clause 107. A dose guidance system comprising a processor and a memory, the memory comprising an algorithm which, which executed by the processor, causes the processor to perform the method of any one of clauses 99 to 106.
Clause 108. A non-transitory computer-readable medium comprising an algorithm which, when executed by a computer, causes the computer to perform the method of any one of clauses 99 to 106.
Clause 109. A method of titrating a medication dose, the method comprises: titrating, by a processor, a medication dose based on glucose data from a first preceding period of time, wherein the glucose data is received from a glucose monitoring device in communication with the processor;
determining, by the processor, that the medication dose is fully titrated based on one or more full titration criteria; and
titrating, by the one or more processors, the medication dose based on glucose data collected over a second preceding period of time when the medication dose is fully titrated as determined based on the one or more full titration criteria, wherein the second preceding period of time is longer than the first preceding period of time.
Clause 110. The method of clause 109, further comprising outputting, by a display device in communication with the processor, a notification when a full titration criterion of the one or more full titration criteria are satisfied.
Clause 111. The method of clause 109 or 110, further comprising stopping titration of the medication dose when a risk of hypoglycemia is detected.
Clause 112. The method of clause 111, wherein the risk of hypoglycemia is based on a time below a predetermined low glucose value exceeding a threshold amount of time.
Clause 113. The method of any one of clauses 109 to 112, further comprising determining that a new therapy is initiated, and stopping titration of the medication dose when the new therapy is initiated.
Clause 114. The method of clause 113, wherein determining that the new therapy is initiated comprises receiving user input indicating initiation of a second medication.
Clause 115. The method of any one of clauses 109 to 114, further comprising determining the medication dose is fully titrated when the medication dose has not changed over a predetermined period of time.
Clause 116. The method of any one of clauses 109 to 115, wherein determining the medication dose is fully titrated comprises determining a glucose pattern for each of a plurality of time of day periods, and determining the medication dose is fully titrated when the determined glucose patterns for the time of day periods have not changed over a predetermined period of time.
Clause 117. The method of any one of clauses 109 to 116, wherein determining the medication dose is fully titrated comprises determining that a change in time in range from a first medication dose to a second medication dose resulted in a change in time in range of less than a predetermined change in time in range threshold.
Clause 118. The method of any one of clauses 109 to 117, wherein determining the medication dose is fully titrated comprises determining that a minimum 4^{th} percentile glucose value is less than a predetermined threshold.
Clause 119. A dose guidance system comprising a processor and a memory, the memory comprising an algorithm which, which executed by the processor, causes the processor to perform the method of any one of clauses 109 to 118.
Clause 120. A non-transitory computer-readable medium comprising an algorithm which, when executed by a computer, causes the computer to perform the method of any one of clauses 109 to 118.
Clause 121. A method for titrating a medication dose, the method comprising: receiving, by a processor, glucose data over a titration period;
setting, by the processor, an initial state estimate comprising a probability that the glucose data corresponds to a time of day period of a plurality of time of day periods;
receiving, by the processor, event data comprising one or more meals or insulin doses recorded during the titration period;
determining, by the processor, corrected state estimates based on the initial state estimate and the event data;
segmenting, by the processor, the received glucose data into a plurality of glucose data segments corresponding to each of the plurality of time of day periods;
titrating, by the processor, an insulin dose based on the glucose data segments for the corresponding time of day period; and
outputting on a display in communication with the processor the titrated insulin dose.
Clause 122. The method of clause 121, wherein the plurality of time of day periods comprises a plurality of meal periods, wherein the plurality of meal periods optionally comprises a first meal period, a second meal period, and a third meal period, and optionally wherein the first meal period is a breakfast period, the second meal period is a lunch period, and the third meal period is a dinner period.
Clause 123. The method of clause 121 or 122, wherein the corrected state estimate is determined based on a model comprising a sequence of the plurality of time of day periods over time.
Clause 124. The method of any one of clauses 121 to 123, wherein each of the plurality of time periods comprises a predetermined duration.
Clause 125. The method of any one of clauses 121 to 124, wherein the event data comprises an insulin dose for a first meal.
Clause 126. The method of clause 125, wherein determining the corrected state estimates comprises determining that a state at a time of the insulin dose for the first meal corresponds to a first time of day period.
Clause 127. The method of clause 125 or 126, wherein determining the corrected state estimates comprises determining that a time of day period preceding the insulin dose for the first meal comprises a second time of day period.
Clause 128. The method of any one of clauses 125 to 127, wherein determining the corrected state estimate comprises determining that a time of day period following the first time of day period is a third time of day period.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the claims and their equivalents.

1.-102. (canceled)

103. A method of titrating an insulin dose, the method comprising:
receiving, by a processor in communication with a glucose monitoring device, glucose data collected by the glucose monitoring device over a first period of time, wherein the glucose monitoring device comprises a first portion positioned below a skin surface of a user, and a second portion positioned above the skin surface of the user;
determining, by the processor, to increase, decrease or maintain the insulin dose based on the glucose data collected over the first period of time;
determining, by the processor, that the insulin dose is fully titrated based on one or more full titration criteria;
performing an action, by the processor, when the processor determines that the insulin dose is fully titrated; and
outputting a recommendation for the insulin dose based on the determination to increase, decrease or maintain the insulin dose when the processor determines that the insulin dose is not fully titrated.

104. The method of claim 103, further comprising repeating titrating the insulin dose over a second period of time when the processor determines that the insulin dose is not fully titrated.

105. The method of claim 104, wherein repeating titrating the insulin dose comprises receiving glucose data over a second period of time, and determining to increase, decrease, or maintain the insulin dose based on the glucose data received over the second period of time.

106. The method of claim 103, wherein the action comprises outputting, on a display device in communication with the processor, a notification indicating that the insulin dose is fully titrated.

107. The method of claim 103, wherein the action comprises repeating titration of the insulin dose over a second period of time following the first period of time by receiving glucose data over the second period of time, and determining to increase, decrease, or maintain the insulin dose based on the glucose data received during the second period of time, wherein the second period of time is longer than the first period of time.

108. The method of claim 103, wherein the action comprises outputting a recommendation to initiate a new medication on a display of a display device in communication with the processor.

109. The method of claim 103, wherein the action comprises preventing, by the processor, further titration of the insulin dose.

110. The method of claim 103, wherein the one or more full titration criteria comprises a determination that the insulin dose has not changed over a predetermined period of time.

111. The method of claim 103, further comprising determining a glucose pattern for each of a plurality of time of day periods based on the glucose data received during the first period of time, and determining the insulin dose is fully titrated when the determined glucose pattern for a time of day period of the plurality of time of day periods has not changed over a predetermined period of time.

112. The method of claim 103, wherein the one or more full titration criteria comprises a determination that a change in a time in range based on the glucose data collected over the first period of time is less than a predetermined change from a time in range determined based on glucose data collected over a period of time preceding the first period of time.

113. The method of claim 103, wherein the one or more full titration criteria comprises a determination that a glucose median based on the glucose data collected during the first period of time is within a predetermined amount of a glucose median goal.

114. A system for titrating an insulin dose, the system comprising:
a glucose monitoring device, comprising:
a glucose sensor comprising a first portion configured to be positioned under a skin surface of a user for detecting glucose levels in a bodily fluid, and a second portion configured to be arranged above the skin surface of the user, and
sensor electronics coupled to the second portion of the glucose sensor and configured to communicate glucose data; and
one or more processors in communication with the glucose monitoring device, a memory coupled to the one or more processors and storing instructions that, when executed by the one or more processors, cause the one or more processors to:
receive glucose data collected by the glucose monitoring device over a first period of time;
determine to increase, decrease or maintain the insulin dose based on the glucose data collected over the first period of time;
determine whether the insulin dose is fully titrated based on one or more full titration criteria;
perform an action when the insulin dose is fully titrated; and
output the recommendation to increase, decrease or maintain the insulin dose when the insulin dose is not fully titrated.

115. The system of claim 114, further comprising repeating titrating the insulin dose over a second period of time when the insulin dose is not fully titrated.

116. The system of claim 114, wherein the action comprises outputting, on a display device in communication with the one or more processors, a notification indicating that the insulin dose is fully titrated.

117. The system of claim 114, wherein the action comprises repeating titration of the insulin dose over a second period of time following the first period of time by receiving glucose data over the second period of time, and determining to increase, decrease, or maintain the insulin dose based on the glucose data collected during the second period of time, wherein the second period of time is longer than the first period of time.

118. The system of claim 114, wherein the action comprises outputting a recommendation to initiate a new medication on a display device in communication with the one or more processors.

119. The system of claim 114, wherein the action comprises preventing further titration of the insulin dose.

120. The system of claim 114, wherein the one or more full titration criteria comprises a determination that the insulin dose has not changed over a predetermined period of time.

121. The system of claim 114, wherein the one or more processors are further caused to determine a glucose pattern for each of a plurality of time of day periods, and wherein the one or more full titration criteria comprises a determination that the glucose pattern for a time of day period of the plurality of time of day periods has not changed over a predetermined period of time.

122. The system of claim 114, wherein the determination to increase, decrease or maintain the insulin dose is further based on a glucose level goal that is selectable by the user.
